(19) United States
(12) Patent Application Publication
(10) Pub. No.: US 2015/0148345 A1
(43) Pub. Date: May 28, 2015

(54) THERAPIES FOR TREATING MYELOPROLIFERATIVE DISORDERS

(71) Applicant: Gilead Sciences, Inc., Foster City, CA (US)

(72) Inventors: Brian Lannutti, San Diego, CA (US); Sarah Meadows, Seattle, WA (US); Christophe Queva, Bainbridge Island, WA (US); Matthew Robert Warr, Madison, CT (US); James Andrew Whitney, Guilford, CT (US)

(21) Appl. No.: 14/553,825

(22) Filed: Nov. 25, 2014

Related U.S. Application Data
(60) Provisional application No. 61/909,072, filed on Nov. 26, 2013.

(51) Int. Cl. A61K 31/52; A61K 45/06; A61K 31/5377; A61K 31/519

(52) U.S. Cl. CPC A61K 31/52; A61K 31/519; A61K 45/06; A61K 31/5377
USPC 514/235.8; 514/263.21; 435/184

(57) ABSTRACT

Provided herein are methods, compositions, and kits for treating myeloproliferative disorders or neoplasms, including polycythemia vera, primary myelofibrosis, thrombocythemia, and essential thrombocythemia.

### CROSS-REFERENCE TO RELATED APPLICATIONS

This application claims benefit under 35 U.S.C. §119(e) to U.S. Provisional Application Ser. No. 61/909,072, filed on Nov. 26, 2013, the entirety of which is incorporated herein by reference.

### FIELD

The present application provides the therapeutics and compositions for treating myeloproliferative disorders or neoplasms. The application also provides the methods for preparation of the compositions, the article of manufacture, and the kit thereof.

### BACKGROUND

Myeloproliferative disorders or neoplasms are caused by genetic defects in the hematopoietic stem cells, resulting in clonal myeloproliferation, bone marrow fibrosis, and abnormal cytokine expression (Tefferi et al). MPN may be classified into four subtypes: chronic myelogenous leukemia (CML), polycythemia vera (PV), essential thrombocythemia (ET), and primary myelofibrosis (PMF). Treatments of myeloproliferative disorders involve allogeneic stem cell transplant. The transplant procedure is preceded by myeloablative chemotherapy, can led to severe treatment-related consequence such as graft-versus-host disease and is limited by performance status, age and donor restrictions.

In 2005, a mutation JAK2V617F in Janus kinase 2 or JAK2, a member of the JAK family of kinases was identified (Baxter et al., Lancet 365:1054-61, 2005; James et al., Nature 434:1144-8, 2005; Kralovics et al., N. Engl. J. Med. 352:1779-90, 2005; Levine et al., Cancer Cell 7:387-97; 2005). The mutation constitutively activates JAK2 and JAK-STAT signaling, resulting in unrestrained cellular proliferation characteristics of myeloproliferative disorders. It is found in the subtypes of PV, ET, and PMF. About 99% of polycythemia vera patients and about 50-60% of essential thrombocytopenia patients and idiopathic myelofibrosis patients have the mutation JAK2V617F (Vainchenker et al., Blood 118:1723-35, 2011).

Several JAK inhibitors have been developed for treating myeloproliferative neoplasms, including ruxolitinib (INCB018424) for treating primary myelofibrosis, fedratinib (SAR302503, TG101348) for treating myelofibrosis, and XL019, SB1518 and AZD1480 for treating post-PV/ET myelofibrosis (Sonbol, Ther. Adv. Hematol. 4: 15-35, 2013). Patients treated with JAK inhibitors exhibit clinical improvement of reduced splenomegaly and/or constitutional symptoms. However, certain patients' anemia and thrombocytopenia conditions are aggravated. CYT387 (momelotinib) or N-(cyanomethyl)-4-(2-(4-morpholinophenylamino) pyrimidin-4-yl)benzamide is a different class of JAK inhibitor that provide additional benefits in improving anemia and/or spleen response. It is currently in clinical trials for treating primary myelofibrosis, polycythemia vera (PV), essential thrombocythemia (ET), and post-PV/ET.

The phosphatidylinositol 3-kinase (PI3K) pathway is shown to be dysregulated in certain myeloproliferative diseases (Kamishimoto et al., Cell Signaling 23: 849-56 2011; Huang et al., ASH 2009 Abstract 1896; Vannucchi et al., ASH 2011 Abstract 3835; Khan et al., Leukemia 27:1882-90, 2013). In vitro studies show that mTOR inhibitors, RAD001 or PP242, combined with AZD1480 or ruxolitinib for 10-14 days resulted in reduced colony formation of erythropoietin endogenous erythroid cells from primary myelofibrosis or polycythemia vera patients (Bogani et al., PLOS One 8: e54826; 2013). Additional in vitro studies showed that JAK2 inhibitors, ruxolitinib or TG101348, combined with pan PI3K inhibitors ZSTK474, GDC0941, or NVP-BEZ235, or with PI3K? inhibitor LY294002 had synergistic effect (i.e. combination index less than 0.5) in reducing colony formation of cells from a polycythemia vera patient. However, no synergistic effect was detected for the combination of JAK2 inhibitors ruxolitinib with PI3Kδ inhibitors IC87114 and TG100115 (Choong et al., ASH 2012). There is no report on the effects of PI3K isoform inhibitors, such as PI3Kδ inhibitors, on the myeloproliferative diseases

It is shown that patients who have received chronic ruxolitinib treatment commonly develop disease persistence as shown by the gradual return of splenomegaly and/or constitutional symptoms, the lack of hematologic or molecular remissions, or the loss of clinical improvement (Gotlib, Hematologist, November 2012:11).

Accordingly, there is a need of effective treatment of myeloproliferative disorders including progressive or relapsed disease.

### SUMMARY

Provided herein are methods, compositions, articles of manufacture, and kits for treating a hyperproliferative disorder by using effective amounts of one, two or more therapeutic agents including a phosphatidylinositol 3-kinase delta (PI3Kδ) inhibitor, a Janus kinase (JAK) inhibitor, or the combination thereof. The methods described herein provide a treatment for a myeloproliferative disorder, comprising administering to a patient a therapeutic effective amount of JAK inhibitor and a therapeutic effective amount of PI3K inhibitor.

In one aspect of the application, the JAK inhibitor is selected from the group consisting of ruxolitinib, fedratinib, tofacitinib, baricitinib, lestaurtinib, pacritinib, XL019, AZD1480, INCB039110, LY2784544, BMS911543, NS018, or N-(cyanomethyl)-4-[2-(4-morpholinoanilino)pyrimidin-4-yl]benzamide; or pharmaceutically acceptable salts thereof. In one embodiment, the JAK inhibitor a JAK2 inhibitor ruxolitinib. In other embodiment, the JAK inhibitor is a JAK2 inhibitor N-(cyanomethyl)-4-[2-(4-morpholinoanilino)pyrimidin-4-yl]benzamide or a pharmaceutically acceptable salt thereof. In some aspect, the JAK inhibitors are selected from Decernotinib (or VX-509), GLPG0634, or GLPG0788, or a pharmaceutically acceptable salt thereof.

In additional aspect, the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino)pyrimidine-5-carbonitrile; or a pharmaceutically acceptable salt thereof. In certain embodiment, the PI3K inhibitor is a PI3Kδ inhibitor selected from the group consisting of (S)-2-(1-((9H-purin-6-yl)amino) propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino) pyrimidine-5-carbonitrile; or a pharmaceutically acceptable salt thereof. Such PI3Kδ inhibitor is predominantly the (S)-enantiomer. In other aspect, the PI3K inhibitor is selected from the group of (S)-3-(1-((9H-purin-6-yl)amino)ethyl)-8-chloro-2-phenylisoquinolin-1(2H)-one, (S)-2,4-diamino-6-(cyclopropyl(5,8-dichloro-4-oxo-3-(pyridin-3-yl)-3,4-dihydroquinazolin-2-yl)methylamino)pyrimidine-5-carbonitrile, (S)-2,4-diamino-6-(1-(5-chloro-3-(4-methylpyridin-3-yl)-4-oxo-3,4-dihydroquinazolin-2-yl)ethylamino)pyrimidine-5-carbonitrile, (S)-2,4-diamino-6-(1-(5-chloro-3-(5-fluoro-4-methylpyridin-3-yl)-4-oxo-3,4-dihydroquinazolin-2-yl)ethylamino)pyrimidine-5-carbonitrile, (S)-2,4-diamino-6-(1-(5-chloro-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-fluoropyridin-3-yl)-4-oxo-3,4-dihydroquinazolin-2-yl)ethylamino)pyrimidine-5-carbonitrile, (S)-2,4-diamino-6-(1-(5-methyl-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-methylpyridin-3-yl)-4-oxo-3,4-dihydroquinazolin-2-yl)ethylamino)pyrimidine-5-carbonitrile, (S)-2,4-diamino-6-((5-chloro-3-(5-fluoropyridin-3-yl)-4-oxo-3,4-dihydroquinazolin-2-yl)(cyclopropyl)methylamino) pyrimidine-5-carbonitrile, (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile, (S)-2,4-diamino-6-(1-(5,8-dichloro-4-oxo-3-(pyridin-3-yl)-3,4-dihydroquinazolin-2-yl)ethylamino) pyrimidine-5-carbonitrile, (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile, (S)-2,4-diamino-6-(1-(5-chloro-8-fluoro-3-(5-fluoropyridin-3-yl)-4-oxo-3,4-dihydroquinazolin-2-yl)ethylamino) pyrimidine-5-carbonitrile, (S)-2,4-diamino-6-((1-(5-chloro-3-(3-cyanophenyl)-4-oxo-3,4-dihydroquinazolin-2-yl)ethyl)amino)pyrimidine-5-carbonitrile, (S)-2,4-diamino-6-((1-(3-(3-cyanophenyl)-6-fluoro-4-oxo-3,4-dihydroquinazolin-2-yl)ethyl)amino)pyrimidine-5-carbonitrile, (S)-2,4-diamino-6-((1-(8-chloro-4-oxo-3-phenyl-3,4-dihydroquinazolin-2-yl)ethyl)amino)pyrimidine-5-carbonitrile, (S)-2,4-diamino-6-((1-(3-(3,5-difluorophenyl)-5,6-difluoro-4-oxo-3,4-dihydroquinazolin-2-yl)ethyl)amino)pyrimidine-5-carbonitrile, (S)-2,4-diamino-6-((1-(3-(3,5-difluorophenyl)-4-oxo-3,4-dihydroquinazolin-2-yl)propyl)amino) pyrimidine-5-carbonitrile, (S)-2,4-diamino-6-((1-(3-(3-cyanophenyl)-5-(difluoromethyl)-4-oxo-3,4-dihydroquinazolin-2-yl)ethyl)amino)pyrimidine-5-carbonitrile, or a pharmaceutically acceptable salt thereof.

The method of the present application comprises administering to a patient in need thereof with N-(cyanomethyl)-4-[2-(4-morpholinoanilino) pyrimidin-4-yl]benzamide, or a pharmaceutically acceptable salt thereof, at a dose between 50 to 350 mg, between 100 to 200 mg or between 150 mg to 300 mg. The method also comprises administering to a patient in need thereof with (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino) pyrimidine-5-carbonitrile; or a pharmaceutically acceptable salt thereof at a dose between 10 mg and 300 mg, between 25 mg and 150 mg, or between 20 mg and 100 mg. Additionally, the method comprises administering to a patient in need thereof with (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl) (cyclopropyl)methyl)amino)pyrimidine-5-carbonitrile; or a pharmaceutically acceptable salt thereof at a dose between 1 mg and 400 mg, between 2 mg and 150 mg, between 5 mg and 100 mg, or between 10 mg and 50 mg. The method also comprises administering to a patient in need thereof with (S)-3-(1-((9H-purin-6-yl)amino)ethyl)-8-chloro-2-phenylisoquinolin-1(2H)-one, (S)-2,4-diamino-6-(cyclopropyl(5,8-dichloro-4-oxo-3-(pyridin-3-yl)-3,4-dihydroquinazolin-2-yl)methylamino)pyrimidine-5-carbonitrile, (S)-2,4-diamino-6-(1-(5-chloro-3-(4-methylpyridin-3-yl)-4-oxo-3,4-dihydroquinazolin-2-yl)ethylamino)pyrimidine-5-carbonitrile, (S)-2,4-diamino-6-(1-(5-chloro-3-(5-fluoro-4-methylpyridin-3-yl)-4-oxo-3,4-dihydroquinazolin-2-yl)ethylamino)pyrimidine-5-carbonitrile, (S)-2,4-diamino-6-(1-(5-chloro-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-fluoropyridin-3-yl)-4-oxo-3,4-dihydroquinazolin-2-yl)ethylamino)pyrimidine-5-carbonitrile, (S)-2,4-diamino-6-(1-(5-methyl-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-methylpyridin-3-yl)-4-oxo-3,4-dihydroquinazolin-2-yl)ethylamino)pyrimidine-5-carbonitrile, (S)-2,4-diamino-6-((5-chloro-3-(5-fluoropyridin-3-yl)-4-oxo-3,4-dihydroquinazolin-2-yl)(cyclopropyl)methylamino) pyrimidine-5-carbonitrile, (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile, (S)-2,4-diamino-6-(1-(5,8-dichloro-4-oxo-3-(pyridin-3-yl)-3,4-dihydroquinazolin-2-yl)ethylamino) pyrimidine-5-carbonitrile, (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile, (S)-2,4-diamino-6-(1-(5-chloro-8-fluoro-3-(5-fluoropyridin-3-yl)-4-oxo-3,4-dihydroquinazolin-2-yl)ethylamino) pyrimidine-5-carbonitrile, (S)-2,4-diamino-6-((1-(5-chloro-3-(3-cyanophenyl)-4-oxo-3,4-dihydroquinazolin-2-yl)ethyl)amino)pyrimidine-5-carbonitrile, (S)-2,4-diamino-6-((1-(3-(3-cyanophenyl)-6-fluoro-4-oxo-3,4-dihydroquinazolin-2-yl)ethyl)amino)pyrimidine-5-carbonitrile, (S)-2,4-diamino-6-((1-(8-chloro-4-oxo-3-phenyl-3,4-dihydroquinazolin-2-yl)ethyl)amino)pyrimidine-5-carbonitrile, (S)-2,4-diamino-6-((1-(3-(3,5-difluorophenyl)-5,6-difluoro-4-oxo-3,4-dihydroquinazolin-2-yl)ethyl)amino)pyrimidine-5-carbonitrile, (S)-2,4-diamino-6-((1-(3-(3,5-difluorophenyl)-4-oxo-3,4-dihydroquinazolin-2-yl)propyl)amino) pyrimidine-5-carbonitrile, (S)-2,4-diamino-6-((1-(3-(3-cyanophenyl)-5-(difluoromethyl)-4-oxo-3,4-dihydroquinazolin-2-yl)ethyl)amino)pyrimidine-5-carbonitrile; or a pharmaceutically acceptable salt thereof at a dose between 10 mg and 300 mg, between 25 mg and 150 mg, or between 20 mg and 100 mg. The method also comprises administering to a patient in need thereof with (S)-2-(1-((9H-purin-6-yl)amino)propyl)-5-fluoro-3-phenylquinazolin-4(3H)-one or (S)-2-(1-((9H-purin-6-yl)amino)ethyl)-6-fluoro-3-phenylquinazolin-4(3H)-one at a dose between 50 mg and 400 mg or between 50 mg and 150 mg. The JAK inhibitor may be administered prior to the PI3K inhibitor, concurrent with the PI3K inhibitor, or subsequent to the PI3K inhibitor. The JAK inhibitor is administered orally, once or twice daily, in a form of tablet, pills, or capsules. In addition, the PI3K inhibitor is administered orally, once or twice daily, in a form of tablet, pills, or capsules.

The method of treating myeloproliferative diseases further comprises one or more therapeutic agents selected from a spleen tyrosine kinase (SYK) inhibitor, a Bruton's tyrosine kinase (BTK) inhibitor, a bromodomain-containing protein (BRD) inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. Additional methods include the one or more therapeutic agent selected from a PI3K (including PI3Kγ, PI3Kδ, PI3Kβ, and PI3Kα) inhibitor, a JAK (including JAK1 and JAK2) inhibitor, a SYK inhibitor, a BTK inhibitor, a BRD (including BRD4 inhibitor), a LOXL (including LOXL1, LOXL2, LOXL3, LOXL4, or LOXL5) inhibitor, a MMP (including MMP2 and MMP9) inhibitor, a A2B inhibitor, an IDH (including IDH1) inhibitor, an ASK (including ASK1) inhibitor, a TPL2 inhibitor, a DDR (including DDR1 and DDR2) inhibitor, a HDAC inhibitor, a PKC inhibitor, or any combination thereof. In some aspect, one or more therapeutic agents are selected from an Abl inhibitor, an ACK inhibitor, an A2B inhibitor, an ASK inhibitor, an Aurora kinase inhibitor, a BTK inhibitor, a BRD inhibitor, a c-Kit inhibitor, a c-Met inhibitor, a CAK inhibitor, a CaMK inhibitor, a CDK inhibitor, a CK inhibitor, a DDR inhibitor, an EGFR inhibitor, a FAK inhibitor, a Flt-3 inhibitor, a FYN inhibitor, a GSK inhibitor, a HCK inhibitor, a HDAC inhibitor, an IKK inhibitor, an IDH inhibitor, an IKK inhibitor, a KDR inhibitor, a LCK inhibitor, a LOX inhibitor, a LOXL inhibitor, a LYN inhibitor, a MMP inhibitor, a MEK inhibitor, a MAPK inhibitor, a NEK9 inhibitor, a NPM-ALK inhibitor, a p38 kinase inhibitor, a PDGF inhibitor, a PK inhibitor, a PLK inhibitor, a PK inhibitor, a PYK inhibitor, a SYK inhibitor, a TPL2 inhibitor, a STK inhibitor, a STAT inhibitor, a SRC inhibitor, a TBK inhibitor, a TIE inhibitor, a TK inhibitor, a VEGF inhibitor, a YES inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

The myeloproliferative disorder is selected from the group consisting of polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS) and systemic mast cell disease (SMCD). In one aspect, the myeloproliferative disorder is myelofibrosis (MF).

In other aspect of the application, a treatment is provided for patients having myeloproliferative disorder selected from the group consisting of polycythemia vera (PV), primary myelofibrosis (PMF), or essential thrombocythemia (ET). The patient has received prior treatment and/or develops disease persistence to treatment of myeloproliferative disorder, or has not previously been treated for myeloproliferative disorder. In additional aspect of the application, a treatment is provided for patients having diseases selected from diffuse large B-cell lymphoma.

In some other aspect, a method for decreasing cell viability, decreasing proliferation, or increasing apoptosis is provided. Such methods comprise contacting cells with an effective amount of JAK inhibitor and an effective amount of PI3K inhibitor. The JAK inhibitor is selected from the group consisting of ruxolitinib, fedratinib, tofacitinib, baricitinib, lestaurtinib, pacritinib, XL019, AZD1480, INCB039110, LY2784544, BMS911543, NS018, or N-(cyanomethyl)-4-[2-(4-morpholinoanilino)pyrimidin-4-yl]benzamide; or pharmaceutically acceptable salts thereof. Also, the PI3K inhibitor is selected from the group of XL147, BKM120, GDC-0941, BAY80-6946, PX-866, CH5132799, XL756, BEZ235, GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino) pyrimidine-5-carbonitrile; or a pharmaceutically acceptable salt thereof. Moreover, the PI3K inhibitor is selected from (S)-3-(1-((9H-purin-6-yl)amino)ethyl)-8-chloro-2-phenylisoquinolin-1(2H)-one, (S)-2,4-diamino-6-(cyclopropyl(5,8-dichloro-4-oxo-3-(pyridin-3-yl)-3,4-dihydroquinazolin-2-yl)methylamino)pyrimidine-5-carbonitrile, (S)-2,4-diamino-6-(1-(5-chloro-3-(4-methylpyridin-3-yl)-4-oxo-3,4-dihydroquinazolin-2-yl)ethylamino) pyrimidine-5-carbonitrile, (S)-2,4-diamino-6-(1-(5-chloro-3-(5-fluoro-4-methylpyridin-3-yl)-4-oxo-3,4-dihydroquinazolin-2-yl)ethylamino)pyrimidine-5-carbonitrile, (S)-2,4-diamino-6-(1-(5-chloro-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-fluoropyridin-3-yl)-4-oxo-3,4-dihydroquinazolin-2-yl)ethylamino)pyrimidine-5-carbonitrile, (S)-2,4-diamino-6-(1-(5-methyl-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-methylpyridin-3-yl)-4-oxo-3,4-dihydroquinazolin-2-yl)ethylamino)pyrimidine-5-carbonitrile, (S)-2,4-diamino-6-((5-chloro-3-(5-fluoropyridin-3-yl)-4-oxo-3,4-dihydroquinazolin-2-yl)(cyclopropyl)methylamino) pyrimidine-5-carbonitrile, (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile, (S)-2,4-diamino-6-(1-(5,8-dichloro-4-oxo-3-(pyridin-3-yl)-3,4-dihydroquinazolin-2-yl)ethylamino) pyrimidine-5-carbonitrile, (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile, (S)-2,4-diamino-6-(1-(5-chloro-8-fluoro-3-(5-fluoropyridin-3-yl)-4-oxo-3,4-dihydroquinazolin-2-yl)ethylamino) pyrimidine-5-carbonitrile, (S)-2,4-diamino-6-((1-(5-chloro-3-(3-cyanophenyl)-4-oxo-3,4-dihydroquinazolin-2-yl)ethyl)amino)pyrimidine-5-carbonitrile, (S)-2,4-diamino-6-((1-(3-(3-cyanophenyl)-6-fluoro-4-oxo-3,4-dihydroquinazolin-2-yl)ethyl)amino)pyrimidine-5-carbonitrile, (S)-2,4-diamino-6-((1-(8-chloro-4-oxo-3-phenyl-3,4-dihydroquinazolin-2-yl)ethyl)amino)pyrimidine-5-carbonitrile, (S)-2,4-diamino-6-((1-(3-(3,5-difluorophenyl)-5,6-difluoro-4-oxo-3,4-dihydroquinazolin-2-yl)ethyl)amino)pyrimidine-5-carbonitrile, (S)-2,4-diamino-6-((1-(3-(3,5-difluorophenyl)-4-oxo-3,4-dihydroquinazolin-2-yl)propyl)amino) pyrimidine-5-carbonitrile, (S)-2,4-diamino-6-((1-(3-(3-cyanophenyl)-5-(difluoromethyl)-4-oxo-3,4-dihydroquinazolin-2-yl)ethyl)amino)pyrimidine-5-carbonitrile; or a pharmaceutically acceptable salt thereof. The method uses cells that are isolated from a subject having myeloproliferative disorder selected from the group consisting of polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS) and systemic mast cell disease (SMCD). Also, the methods uses cells that are isolated from a subject having diffuse large B-cell lymphoma (DLBCL).

In some aspect, a pharmaceutical composition comprising a therapeutically effective amount of JAK inhibitor, a therapeutically effective amount of PI3K inhibitor, and a pharmaceutically acceptable excipient is provided.

In certain aspect, a kit comprising a pharmaceutical composition and a label is provided. The kit contains the pharmaceutical composition that comprises a therapeutically effective amount of JAK inhibitor, a therapeutically effective amount of PI3K inhibitor, and a pharmaceutically acceptable excipient.

In one aspect the application provides a JAK inhibitor and a PI3K inhibitor for use in a method for treating a myeloproliferative disorder. In one aspect the application provides a JAK2 inhibitor N-(cyanomethyl)-4-[2-(4-morpholinoanilino)pyrimidin-4-yl]benzamide; or a pharmaceutically acceptable salt thereof, which is administered at a dose between 50 to 350 mg; or between 100 to 200 mg. In one aspect the application provides a PI3Kδ inhibitor selected from the group consisting of (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino)pyrimidine-5-carbonitrile; or a pharmaceutically acceptable salt thereof. In an additional aspect, the PI3Kδ inhibitor is predominantly the (S)-enantiomer. In an additional aspect the PI3Kδ inhibitor is administered at a dose between 10 mg and 300 mg, or between 25 mg and 150 mg. In one aspect the method of treating myeloproliferative diseases further comprises one or more therapeutic agents selected from a SYK inhibitor, a BTK inhibitor, a BRD inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. In one aspect, the administration of the JAK inhibitor is prior to the administration of the PI3K inhibitor. In another aspect, the administration of the JAK inhibitor is concurrent to the administration of the PI3K inhibitor. In another aspect, the administration of the JAK inhibitor is subsequent to the administration of the PI3K inhibitor.

In certain aspect, the application provides a JAK inhibitor and a PI3K inhibitor for use in a method for treating a hyperproliferative disorder. In some aspect, the application provides a JAK inhibitor and a PI3K-δ inhibitor for use in a method for treating a hyperproliferative disorder. In additional aspect, the application provides a PI3K inhibitor for use in a method for treating a hyperproliferative disorder. In other aspect, the application provides a PI3K-δ inhibitor for use in a method for treating a hyperproliferative disorder. In one aspect, the hyperproliferative disorder is myeloproliferative disorder. In other aspect, the hyperproliferative disorder is cancer. In additional aspect, the application provides a PI3K inhibitor for use in treating hyperproliferative disorders or neoplasms, wherein the PI3K inhibitor is administered simultaneously, separately or sequentially with a PI3K inhibitor.

In one aspect, the method of treating hyperproliferative diseases comprising administering a therapeutically effective amount of an Abl inhibitor, an ACK inhibitor, an A2B inhibitor, an ASK inhibitor, an Aurora kinase inhibitor, a BTK inhibitor, a BRD inhibitor, a c-Kit inhibitor, a c-Met inhibitor, a CAK inhibitor, a CaMK inhibitor, a CDK inhibitor, a CK inhibitor, a DDR inhibitor, an EGFR inhibitor, a FAK inhibitor, a Flt-3 inhibitor, a FYN inhibitor, a GSK inhibitor, a HCK inhibitor, a HDAC inhibitor, an IKK inhibitor, an IDH inhibitor, an IKK inhibitor, a JAK inhibitor, a KDR inhibitor, a LCK inhibitor, a LOX inhibitor, a LOXL inhibitor, a LYN inhibitor, a MMP inhibitor, a MEK inhibitor, a MAPK inhibitor, a NEK9 inhibitor, a NPM-ALK inhibitor, a p38 kinase inhibitor, a PDGF inhibitor, a PI3 kinase (PI3K), a PK inhibitor, a PLK inhibitor, a PK inhibitor, a PYK inhibitor, a SYK inhibitor, a TPL2 inhibitor, a STK inhibitor, a STAT inhibitor, a SRC inhibitor, a TBK inhibitor, a TIE inhibitor, a TK inhibitor, a VEGF inhibitor, a YES inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. In certain aspect, the one or more therapeutic agent is selected from a PI3K (including PI3Kγ, PI3Kδ, PI3Kβ, and PI3Kα) inhibitor, a JAK (including JAK1 and JAK2) inhibitor, a SYK inhibitor, a BTK inhibitor, a BRD (including BRD4 inhibitor), a chemotherapeutic agent, an immunotherapeutic agent, a radiotherapeutic agent, an anti-neoplastic agent, an anti-cancer agent, an anti-proliferation agent, or any combination thereof.

In some aspect, the application provides a JAK inhibitor and a PI3K-δ inhibitor for use in a method for treating a myeloproliferative disorder. In additional aspect, the application provides a PI3K inhibitor for use in a method for treating a myeloproliferative disorder. In other aspect, the application provides a PI3K-δ inhibitor for use in a method for treating a myeloproliferative disorder. In other aspect, the administration of the JAK inhibitor is prior to the administration of the PI3K inhibitor. In one aspect the application provides a JAK2 inhibitor N-(cyanomethyl)-4-[2-(4-morpholinoanilino)pyrimidin-4-yl]benzamide; or a pharmaceutically acceptable hydrochloride salt thereof, which is administered at a dose between 100 to 300 mg. In additional aspect, the application provides a JAK inhibitor ruxolitinib, or a pharmaceutically acceptable phosphate salt thereof, which is administered at a dose between 15 to 25 mg. In one aspect the application provides a PI3K inhibitor selected from the group consisting of (S)-2-(1-((9H-purin-6-yl)amino)propyl)-5-fluoro-3-phenylquinazolin-4(3H)-one, which is administered at a dose between 50 mg and 150 mg.

In one aspect, the application provides 2-(1-((9H-purin-6-yl)amino)propyl)-5-fluoro-3-phenylquinazolin-4(3H)-one, or a pharmaceutically acceptable salt thereof and ruxolitinib or a pharmaceutically acceptable salt thereof for use in a method of treating myeloproliferative disease. In some aspect, the application provides (S)-2-(1-((9H-purin-6-yl)amino)propyl)-5-fluoro-3-phenylquinazolin-4(3H)-one and ruxolitinib, or a pharmaceutical acceptable phosphate salt thereof, for use in a method of treating myeloproliferative disease. In other aspect, the application provides (S)-2-(1-((9H-purin-6-yl)amino)propyl)-5-fluoro-3-phenylquinazolin-4(3H)-one or a pharmaceutically acceptable salt thereof and N-(cyanomethyl)-4-[2-(4-morpholinoanilino)pyrimidin-4-yl]benzamide or a pharmaceutical acceptable salt for use in a method of treating myeloproliferative disease. In some other aspect the application provides (S)-2-(1-((9H-purin-6-yl)amino)propyl)-5-fluoro-3-phenylquinazolin-4(3H)-one and N-(cyanomethyl)-4-[2-(4-morpholinoanilino) pyrimidin-4-yl]benzamide or a pharmaceutical acceptable hydrochloride salt for use in a method of treating myeloproliferative disease. In a further aspect, the myeloproliferative disease is selected from primary myelofibrosis, post-polycythemia or post-essential thrombocythemia myelofibrosis.

In one aspect, the application provides a PI3K inhibitor for use in treating myeloproliferative disorders or neoplasms in a subject (e.g. human) which has received chronic ruxolitinib (e.g. over 3-6 months, more than 6 months, or more than one year). In one embodiment, the PI3K inhibitor is 2-(1-((9H-purin-6-yl)amino)propyl)-5-fluoro-3-phenylquinazolin-4(3H)-one. In a further embodiment, the PI3K inhibitor is (S)-2-(1-((9H-purin-6-yl)amino)propyl)-5-fluoro-3-phenylquinazolin-4(3H)-one. In further aspect, the application provides a use of a PI3K inhibitor for the manufacture of a medicament for treatment of a hyperproliferative disorder. In other aspect, the application provides a use of a JAK inhibitor and a PI3K inhibitor for the manufacture of a medicament for treatment of a hyperproliferative disorder. In additional aspect, the application provides a use of a JAK inhibitor and a PI3K inhibitor for the manufacture of a medicament for treatment of a myeloproliferative disorder. In yet another aspect, the application provides a use of a JAK inhibitor and PI3K inhibitor for the manufacture of a medicament for treatment of a cancer. In other aspect, the application provides a use of a PI3K inhibitor for the manufacture of a medicament for treatment of a myeloproliferative disorder. In one aspect, the application provides a PI3K inhibitor for use in treating myeloproliferative disorders or neoplasms, wherein the PI3K inhibitor is administered simultaneously, separately or sequentially with a JAK inhibitor.

In one aspect, the application provides a product containing a JAK inhibitor and a PI3K inhibitor as a combined preparation for simultaneous, separate or sequential use in treating myeloproliferative disorders or neoplasms.

### DETAILED DESCRIPTION

The following description sets forth exemplary methods, compositions, kits and articles of manufacture for treating myeloproliferative disorders or neoplasm. Such description exemplifies embodiments and does not limit the scope of the present disclosure.

The present application provides methods for treating hyperproliferative disorders such as cancers and myeloproliferative disorders in a subject by administering one or more therapeutic agents. The myeloproliferative disorders (MPD), also referred to as myeloproliferative neoplasms (MPN), are caused by mutations in the hematopoietic (or early myeloid progenitor) stem cells that result in excessive production of myeloid lineage cells (such as bone marrow), clonal myeloproliferation, bone marrow fibrosis, and abnormal cytokine expression. MPN includes, among others, polycythemia vera (PV), primary myelofibrosis, thrombocythemia, essential thrombocythemia (ET), idiopathic myelofibrosis, chronic myelogenous leukemia (CML), systemic mastocystosis, chronic neutrophilic leukemia, myelodysplastic syndrome, and systemic mast cell disease. MPN patients may further develop acute myeloid leukemia (AML), which is often associated with a poor outcome. Current MPN therapies aim at providing palliative care over a long period of time.

The methods provided herein treat myeloproliferative diseases by administering one or more therapeutic agents for treating myeloproliferative diseases. In certain embodiments, the methods use or include a single therapeutic agent. In other embodiment, the methods use or include a combination of two or more therapeutic agents. In some embodiments, a method is provided for treating myeloproliferative diseases by administering a combination of therapeutic agents or small molecule inhibitors that inhibit B-cell receptor (BCR)-mediated signaling, phosphatidylinositol 3-kinase (PI3K)-mediated, Janus kinase (JAK)-mediated signaling pathways, or any combination thereof.

A therapeutic agent may be a compound or a biologic molecule (such as DNA, RNA, or protein) that provide desired therapeutic effects when administered to a subject in need thereof (e.g. MPN patients). For example, the therapeutic agent is a compound that inhibits kinase that, directly or indirectly, relates to the disease mechanism or development. As used herein, enhanced therapeutic effects or variants thereof refer to additional beneficial or synergistic effects to patients that are not observed previously, including fewer and/or reduced symptoms, higher survival rate, prolonged survival time, shorter treatment duration, lower drug dosage, increased molecular and/or cellular responses, and the like.

The combination of therapeutic agents or inhibitors may target upstream or downstream components of the same pathway. Alternatively, the combination of therapeutic agents or inhibitors may target different components of dual or multiple pathways. It is hypothesized that the use of a combination of therapeutic agents or inhibitors may enhance therapeutic effects compared to the use of a single therapeutic agent or inhibitor.

PI3K Class I has the four p110 catalytic subunit isoforms α, β, δ, and γ. PI3K p110 delta isoform is over-expressed in many B-cell malignancies, including CLL. It is shown that the PI3Kδ inhibitors promote apoptosis in B-cell malignancies by disrupting the molecular pathways related to BCR signaling, leukemia cell migration and microenvironment. Also, the PI3Kδ inhibitors inhibits BCR derived PI3K signaling, which leads to inhibition of AKT activation. Without being bound to any theories, a PI3Kδ inhibitor may resensitize or reactivate JAK2 phosphorylation in the JAK-signaling pathway, resulting in increased patient response to prior, concurrent, or subsequent MPN therapies by overcoming drug resistance or disease persistence from the use of a single JAK inhibitor such as ruxolitinib. Alternatively, targeting PI3K p110δ inhibition may result in direct destruction of the diseased cell or repression of microenvironmental signals that are needed for signaling pathways relating to cell survival, proliferation, or hyperproliferation. As described herein, targeting or inhibiting PI3Kδ and JAK provides a novel approach for the treatment of hyperproliferative diseases.

Regardless of the mechanism, such effects are desired in treating hyperproliferative diseases such as cancers and MPN as the treatment is generally provided over a long period of time (i.e. chronic therapies) and drug resistance or disease persistence are commonly observed during chronic therapies. Thus, dual or multiple inhibitions by a combination of two, three or more therapeutic agents may enhance treatment or therapeutic effects in myeloproliferative diseases.

The application also provides compositions (including pharmaceutical compositions, formulations, or unit dosages), articles of manufacture and kits comprising one or more therapeutic agents, including a PI3K inhibitor (including a PI3Kδ inhibitor), a spleen tyrosine kinase (SYK) inhibitor, a Janus kinase (JAK) inhibitor (including a JAK2 inhibitor), a Bruton's tyrosine kinase (BTK) inhibitor, and a bromodomain containing protein inhibitor (BRD) inhibitor (including a BRD4 inhibitor). In some embodiments, one or more therapeutic agent is selected from a PI3K (including PI3Kγ, PI3Kδ, PI3Kβ, PI3Kα, and/or pan-PI3K) inhibitor, a JAK (including JAK1 and/or JAK2) inhibitor, a SYK inhibitor, a BTK inhibitor, an A2B (adenosine A2B receptor) inhibitor, an ACK (activated CDC kinase, including ACK1) inhibitor, an ASK (apoptosis signal-regulating kinase, including ASK1) inhibitor, Aurora kinase, a BRD (bromodomain-containing protein, including BRD4) inhibitor, a CAK (CDK-activating kinase) inhibitor, a CaMK (calmodulin-dependent protein kinases) inhibitor, a CDK (cyclin-dependent kinases, including CDK1, 2, 3, 4, and/or 6) inhibitor, a CK (casein kinase, including CK1 and/or C1(2) inhibitor, a DDR (discoidin domain receptor, including DDR1 and/or DDR2) inhibitor, a EGFR inhibitor, a FAK (focal adhesion kinase) inhibitor, a GSK (glycogen synthase kinase) inhibitor, a HDAC (histone deacetylase) inhibitor, an IDH (isocitrate dehydrogenase, including IDH1) inhibitor, an IKK inhibitor, a LCK (lymphocyte-specific protein tyrosine kinase) inhibitor, a LOX (lysyl oxidase) inhibitor, a LOXL (lysyl oxidase like protein, including LOXL1, LOXL2, LOXL3, LOXL4, and/or LOXL5) inhibitor, a MEK inhibitor, a matrix metalloprotease (MMP, including MMP2 and/or MMP9) inhibitor, a mitogen-activated protein kinases (MAPK) inhibitor, a PDGF (platelet-derived growth factor) inhibitor, a phosphorylase kinase (PK) inhibitor, a PLK (polo-like kinase, including PLK1, 2, 3) inhibitor, a protein kinase (PK, including protein kinase A, B, C) inhibitor, a serine/threonine kinase (STK) inhibitor, a STAT (signal transduction and transcription) inhibitor, a TBK (serine/threonine-protein kinase, including TBK1) inhibitor, a TK (tyrosine kinase) inhibitor, a TPL2 (serine/threonine kinase) inhibitor, a NEK9 inhibitor, an Abl inhibitor, a p38 kinase inhibitor, a PYK inhibitor, a PYK inhibitor, a c-Kit inhibitor, a NPM-ALK inhibitor, a Flt-3 inhibitor, a c-Met inhibitor, a KDR inhibitor, a TIE-2 inhibitor, a VEGFR inhibitor, a SRC inhibitor, a HCK inhibitor, a LYN inhibitor, a FYN inhibitor, a YES inhibitor, or any combination thereof. By way of example, the therapeutic agents include a PI3Kδ inhibitor, or a pharmaceutically acceptable salt thereof, and a JAK2 inhibitor, or a pharmaceutically acceptable salt thereof.

As described in the present application, the administration of a PI3Kδ inhibitor, including (S)-2-(1-(9H-purin-6-ylamino)propyl)-5-fluoro-3-phenylquinazolin-4(3H)-one, (S)-2-(1-((9H-purin-6-yl)amino)ethyl)-6-fluoro-3-phenylquinazolin-4(3H)-one, or (S)-2,4-diamino-6-(((5-chloro-8-fluoro-4-oxo-3-(pyridin-3-yl)-3,4-dihydroquinazolin-2yl)(cyclopropyl)methyl)amino)pyrimidine-5-carbonitrile, and a JAK inhibitor, including N-(cyanomethyl)-4-(2-((4-morpholinophenyl)amino)pyrimidin-4-yl)benzamide or ruxolitinib, to diseased cells or patients has led to unexpected enhanced therapeutic effects compared to the administration of each kinase inhibitor alone. The unexpected synergistic effects include, but are not limited to, for example, decreased cell viability, increased cell death or apoptosis, decreased inhibition or interference with PI3K signaling pathways (including AKT, S6RP, ERK phosphorylation), and/or reduction in chemokine (e.g., CCL2, CCL3, CLL4 and CLL22) production, reduced colony formation in diseased cells or patients. Also, unexpected effects may include, but are not limited to, increased inhibition or interference of JAK/STAT (including STAT3 and STAT5) and/or PI3K/AKT signaling pathways, decreased doses or duration of a single agent treatment. Further, the administration of both PI3Kδ and JAK inhibitors unexpectedly restored or increased sensitivity or response of the diseased cells that had developed resistance or the patients developed disease persistence to prior treatment.

### Therapeutic Agents

The present application provides methods, compositions, kits and articles of manufacture thereof that use or include one or more therapeutic agents inhibiting one or more targets that relate to, directly or indirectly, to cell growth, proliferation, or apoptosis for treating hyperproliferative disorders such as cancers or myeloproliferative neoplasms. The one or more therapeutic agents are compounds or molecules that target a PI3 kinase (PI3K), a spleen tyrosine kinase (SYK), a Janus kinase (JAK), a bromodomain-containing (BRD), a Bruton's tyrosine kinase (BTK), or any combination thereof, resulting in the inhibition of the target. In certain embodiments, the therapeutic agent is a PI3Kδ inhibitor that selectively inhibits PI3K p110 delta isoform (PI3Kδ). In some embodiments, the therapeutic agents are a PI3Kδ inhibitor and a JAK2 inhibitor.

The JAK inhibitor binds and inhibits one or more members of JAK family, including JAK1, JAK2, and/or JAK3. For example, the JAK inhibitor is the compound having the structure of formula (I) shown below.

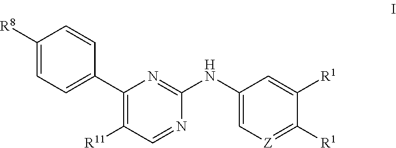

wherein

Z is independently selected from N and CH;

R¹ is independently selected from H, halogen, OH, CONHR², CON(R²)₂, CF₃, R²OR², CN, morpholino, thiomorpholinyl, thiomorpholino-1,1-dioxide, optionally substituted piperidinyl, optionally substituted piperazinyl, imidazolyl, optionally substituted pyrrolidinyl and C₁₋₄alkylene wherein the carbon atoms are optionally substituted with NR^{Y} and/or O substituted with morpholino, thiomorpholinyl, thiomorpholino-1,1-dioxide, optionally substituted piperidinyl, optionally substituted piperazinyl, imidazolyl or optionally substituted pyrrolidinyl;

R² is optionally substituted C₁₋₄alkyl;

R^{Y} is H or optionally substituted C₁₋₄alkyl;

R⁸ is R^{X}CN;

R^{X} is optionally substituted C₁₋₄alkylene wherein up to 2 carbon atoms can be optionally substituted with CO, NSO₂R¹, NR^{Y}, CONR^{Y}, SO, SO₂ or O; and

R¹¹ is H, halogen, C₁₋₄alkyl or C₁₋₄alkyloxy;

or a pharmaceutically acceptable salt thereof.

In one embodiment, the JAK inhibitor is Compound A having the structure:

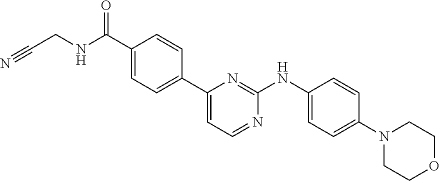 [SMILES: N#CCNC(=O)c1ccc(-c2ccnc(Nc3ccc(N4CCOCC4)cc3)n2)cc1]

In another embodiment, the JAK inhibitor is Compound A in a pharmaceutically acceptable salt thereof.

Compound A may be referred to by its compound name: N-(cyanomethyl)-4-[2-(4-morpholinoanilino)pyrimidin-4-yl]benzamide using ChemDraw. Compound A, also referred to as CYT0387 or momelotinib, is a selective inhibitor to JAK2 and JAK1, relative to JAK3. Methods for synthesizing compounds of formula I and Compound A are previously described in U.S. Pat. No. 8,486,941. This reference is hereby incorporated herein by reference in its entirety.

Additional JAK inhibitors include, but are not limited to, ruxolitinib (INCB018424), fedratinib (SAR302503, TG101348), tofacitinib, baricitinib, lestaurtinib, pacritinib (SB1518), XL019, AZD1480, INCB039110, LY2784544, BMS911543, and NS018. Other JAK inhibitors include, but not limited to, Decemotinib (or VX-509), GLPG0634, or GLPG0788, or a pharmaceutically acceptable salt thereof.

The PI3K inhibitors inhibit to one or more isoforms of Class I PI3K, including PI3Kα, PI3Kβ, PI3Kδ, PI3Kγ, or any combination thereof. For example, the PI3K inhibitor is a PI3Kδ inhibitor having the structure of formula II as shown below.

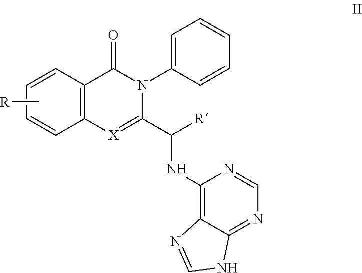

wherein

X is CH or N;

R is H, halo, or C₁₋₆ alkyl; and

R′ is C₁₋₆ alkyl;

or a pharmaceutically acceptable salt thereof.

In some embodiments, the PI3Kδ inhibitor is Compound B having the structure:

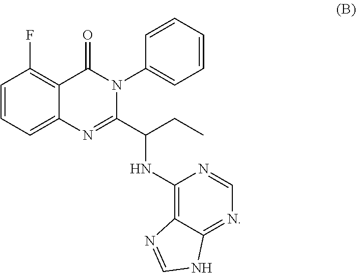 [SMILES: CCC(Nc1ncnc2[nH]cnc12)c1nc2cccc(F)c2c(=O)n1-c1ccccc1]

In other embodiments, Compound B is predominantly the S-enantiomer, having the structure:

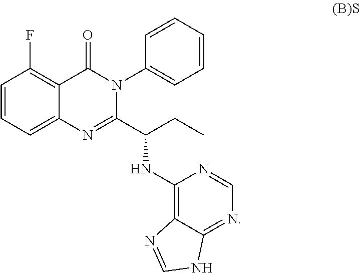 [SMILES: CC[C@H](Nc1ncnc2[nH]cnc12)c1nc2cccc(F)c2c(=O)n1-c1ccccc1]

The (S)-enantiomer of Compound B may also be referred to by its compound name: (S)-2-(1-((9H-purin-6-yl)amino)propyl)-5-fluoro-3-phenylquinazolin-4(3H)-one using ChemDraw.

In certain embodiments, the PI3Kδ inhibitor is Compound C having the structure:

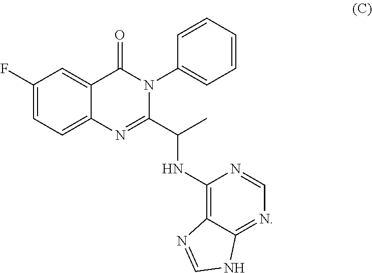 [SMILES: CC(Nc1ncnc2[nH]cnc12)c1nc2ccc(F)cc2c(=O)n1-c1ccccc1]

In additional embodiments, Compound C is predominantly the S-enantiomer, having the structure:

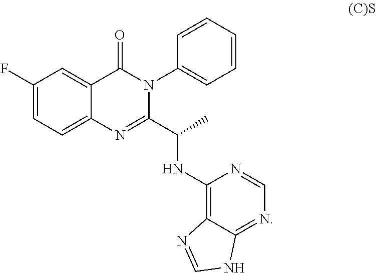 [SMILES: C[C@H](Nc1ncnc2[nH]cnc12)c1nc2ccc(F)cc2c(=O)n1-c1ccccc1]

The (S)-enantiomer of Compound C may also be referred to by its compound name: (S)-2-(1-((9H-purin-6-yl)amino)ethyl)-6-fluoro-3-phenylquinazolin-4(3H)-one using ChemDraw.

In other embodiments, the PI3Kδ inhibitor is Compound D1 having the structure:

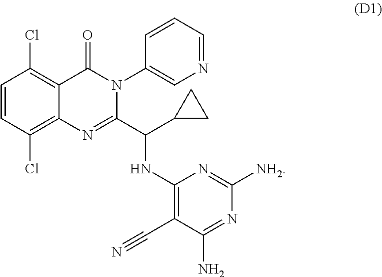 [SMILES: N#Cc1c(N)nc(N)nc1NC(c1nc2c(Cl)ccc(Cl)c2c(=O)n1-c1cccnc1)C1CC1]

In additional embodiments, Compound D1 is predominantly the S-enantiomer, having the structure:

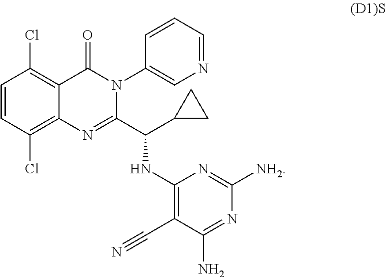 [SMILES: N#Cc1c(N)nc(N)nc1N[C@H](c1nc2c(Cl)ccc(Cl)c2c(=O)n1-c1cccnc1)C1CC1]

The (S) enantiomer of Compound D1 may also be referred to by its compound name: (S)-2,4-diamino-6-(cyclopropyl(5,8-dichloro-4-oxo-3-(pyridin-3-yl)-3,4-dihydroquinazolin-2-yl) methylamino)pyrimidine-5-carbonitrile using ChemDraw.

In some other embodiments, the PI3Kδ inhibitor is Compound D2 having the structure:

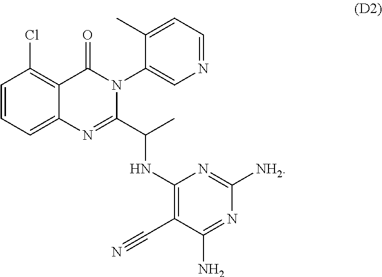 [SMILES: Cc1ccncc1-n1c(C(C)Nc2nc(N)nc(N)c2C#N)nc2cccc(Cl)c2c1=O]

In some additional embodiments, Compound D2 is predominantly the S-enantiomer, having the structure:

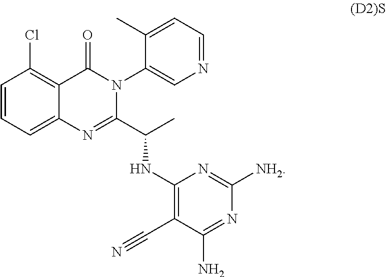 [SMILES: Cc1ccncc1-n1c([C@H](C)Nc2nc(N)nc(N)c2C#N)nc2cccc(Cl)c2c1=O]

The (S) enantiomer of Compound D2 may also be referred to by its compound name: (S)-2,4-diamino-6-(1-(5-chloro-3-(4-methylpyridin-3-yl)-4-oxo-3,4-dihydroquinazolin-2-yl) ethylamino)pyrimidine-5-carbonitrile using ChemDraw.

In certain embodiments, the PI3Kδ inhibitor is Compound D3 having the structure:

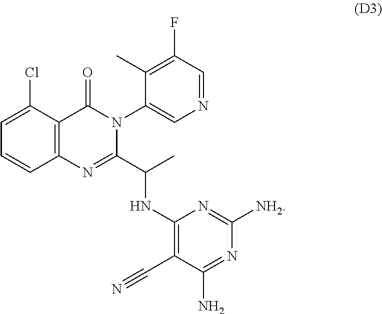 [SMILES: Cc1c(F)cncc1-n1c(C(C)Nc2nc(N)nc(N)c2C#N)nc2cccc(Cl)c2c1=O]

In certain additional embodiments, Compound D3 is predominantly the S-enantiomer, having the structure:

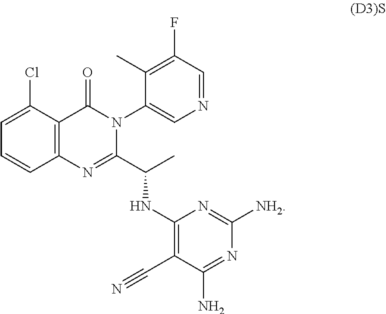 [SMILES: Cc1c(F)cncc1-n1c([C@H](C)Nc2nc(N)nc(N)c2C#N)nc2cccc(Cl)c2c1=O]

The (S) enantiomer of Compound D3 may also be referred to by its compound name: (S)-2,4-diamino-6-(1-(5-chloro-3-(5-fluoro-4-methylpyridin-3-yl)-4-oxo-3,4-dihydroquinazolin-2-yl) ethylamino)pyrimidine-5-carbonitrile using ChemDraw.

In other embodiments, the PI3Kδ inhibitor is Compound D4 having the structure:

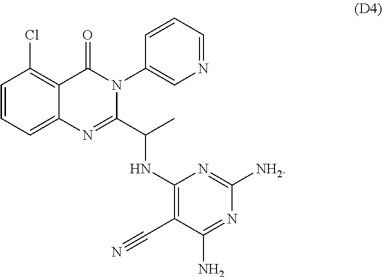 [SMILES: CC(Nc1nc(N)nc(N)c1C#N)c1nc2cccc(Cl)c2c(=O)n1-c1cccnc1]

In other additional embodiments, Compound D4 is predominantly the S-enantiomer, having the structure:

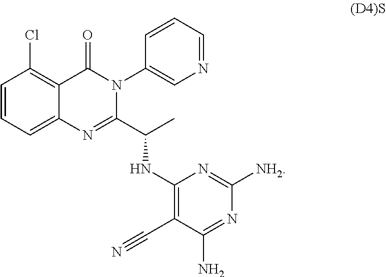 [SMILES: C[C@H](Nc1nc(N)nc(N)c1C#N)c1nc2cccc(Cl)c2c(=O)n1-c1cccnc1]

The (S) enantiomer of Compound D4 may also be referred to by its compound name: (S)-2,4-diamino-6-(1-(5-chloro-4-oxo-3-(pyridin-3-yl)-3,4-dihydroquinazolin-2-yl)ethylamino) pyrimidine-5-carbonitrile using ChemDraw.

In some other embodiments, the PI3Kδ inhibitor is Compound D5 having the structure:

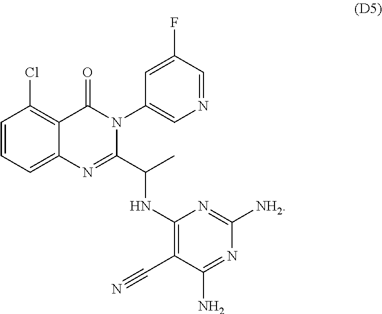 [SMILES: CC(Nc1nc(N)nc(N)c1C#N)c1nc2cccc(Cl)c2c(=O)n1-c1cncc(F)c1]

In additional embodiments, Compound D5 is predominantly the S-enantiomer, having the structure:

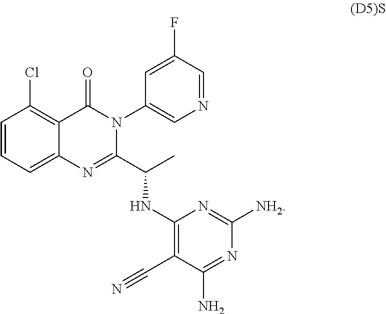 [SMILES: C[C@H](Nc1nc(N)nc(N)c1C#N)c1nc2cccc(Cl)c2c(=O)n1-c1cncc(F)c1]

The (S) enantiomer of Compound D5 may also be referred to by its compound name: (S)-2,4-diamino-6-(1-(5-chloro-3-(5-fluoropyridin-3-yl)-4-oxo-3,4-dihydroquinazolin-2-yl) ethylamino)pyrimidine-5-carbonitrile using ChemDraw.

In other embodiments, the PI3Kδ inhibitor is Compound D6 having the structure:

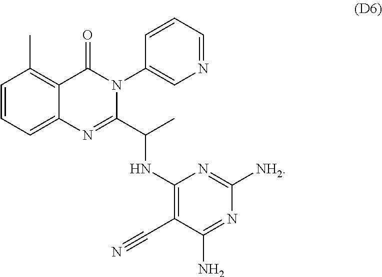 [SMILES: Cc1cccc2nc(C(C)Nc3nc(N)nc(N)c3C#N)n(-c3cccnc3)c(=O)c12]

In additional embodiments, Compound D6 is predominantly the S-enantiomer, having the structure:

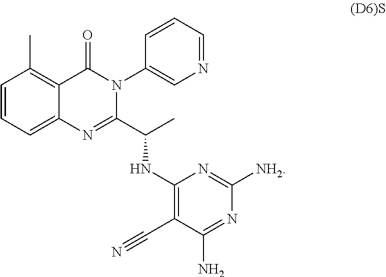 [SMILES: Cc1cccc2nc([C@H](C)Nc3nc(N)nc(N)c3C#N)n(-c3cccnc3)c(=O)c12]

The (S) enantiomer of Compound D6 may also be referred to by its compound name: (S)-2,4-diamino-6-(1-(5-methyl-4-oxo-3-(pyridin-3-yl)-3,4-dihydroquinazolin-2-yl)ethylamino) pyrimidine-5-carbonitrile using ChemDraw.

In some embodiments, the PI3Kδ inhibitor is Compound D7 having the structure:

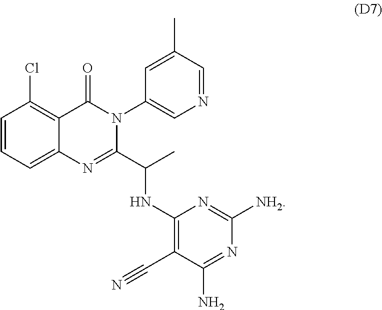 [SMILES: Cc1cncc(-n2c(C(C)Nc3nc(N)nc(N)c3C#N)nc3cccc(Cl)c3c2=O)c1]

In additional embodiments, Compound D7 is predominantly the S-enantiomer, having the structure:

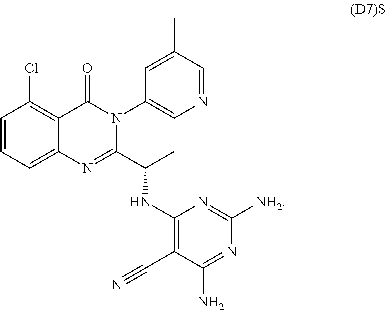 [SMILES: Cc1cncc(-n2c([C@H](C)Nc3nc(N)nc(N)c3C#N)nc3cccc(Cl)c3c2=O)c1]

The (S) enantiomer of Compound D7 may also be referred to by its compound name: (S)-2,4-diamino-6-(1-(5-chloro-3-(5-methylpyridin-3-yl)-4-oxo-3,4-dihydroquinazolin-2-yl) ethylamino)pyrimidine-5-carbonitrile using ChemDraw.

In certain embodiments, the PI3Kδ inhibitor is Compound D8 having the structure:

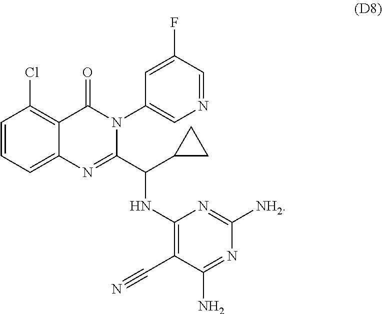 [SMILES: N#Cc1c(N)nc(N)nc1NC(c1nc2cccc(Cl)c2c(=O)n1-c1cncc(F)c1)C1CC1]

In certain additional embodiments, Compound D8 is predominantly the S-enantiomer, having the structure:

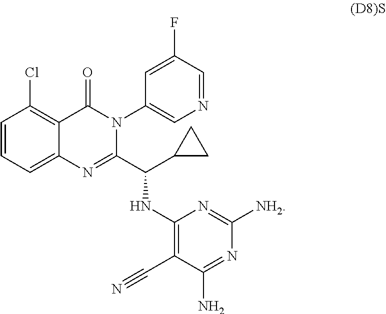 [SMILES: N#Cc1c(N)nc(N)nc1N[C@H](c1nc2cccc(Cl)c2c(=O)n1-c1cncc(F)c1)C1CC1]

The (S) enantiomer of Compound D8 may also be referred to by its compound name: (S)-2,4-diamino-6-((5-chloro-3-(5-fluoropyridin-3-yl)-4-oxo-3,4-dihydroquinazolin-2-yl) (cyclopropyl)methylamino)pyrimidine-5-carbonitrile using ChemDraw.

In some embodiments, the PI3Kδ inhibitor is Compound D9 having the structure:

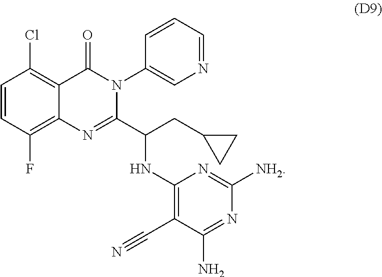 [SMILES: N#Cc1c(N)nc(N)nc1NC(CC1CC1)c1nc2c(F)ccc(Cl)c2c(=O)n1-c1cccnc1]

In some other embodiments, Compound D9 is predominantly the S-enantiomer, having the structure:

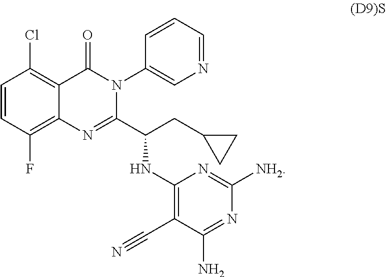 [SMILES: N#Cc1c(N)nc(N)nc1N[C@@H](CC1CC1)c1nc2c(F)ccc(Cl)c2c(=O)n1-c1cccnc1]

The (S) enantiomer of Compound D9 may also be referred to by its compound name: (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile using ChemDraw.

In another embodiment, the PI3K inhibitor is Compound D, having the structure:

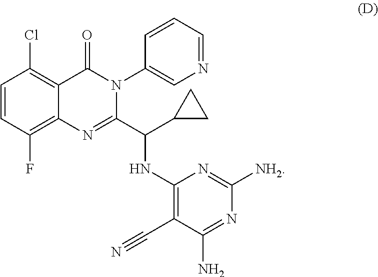 [SMILES: N#Cc1c(N)nc(N)nc1NC(c1nc2c(F)ccc(Cl)c2c(=O)n1-c1cccnc1)C1CC1]

In one embodiment, Compound D is predominantly the S-enantiomer, having the structure:

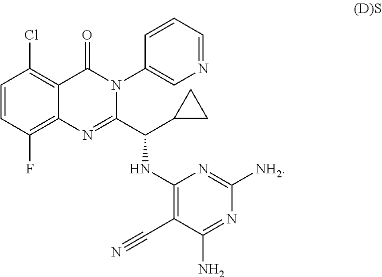 [SMILES: N#Cc1c(N)nc(N)nc1N[C@H](c1nc2c(F)ccc(Cl)c2c(=O)n1-c1cccnc1)C1CC1]

The (S)-enantiomer of Compound D may also be referred to by its compound name: (S)-2,4-diamino-6-(((5-chloro-8-fluoro-4-oxo-3-(pyridin-3-yl)-3,4-dihydroquinazolin-2-yl) (cyclopropyl)methyl)amino)pyrimidine-5-carbonitrile using ChemDraw.

In further embodiments, the PI3Kδ inhibitor is Compound E1 having the structure:

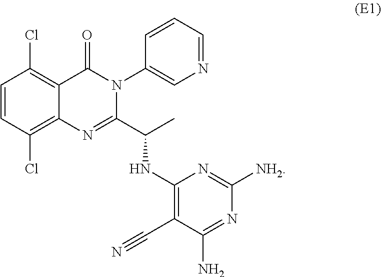 [SMILES: C[C@H](Nc1nc(N)nc(N)c1C#N)c1nc2c(Cl)ccc(Cl)c2c(=O)n1-c1cccnc1]

In additional embodiments, Compound E1 is predominantly the S-enantiomer, having the structure:

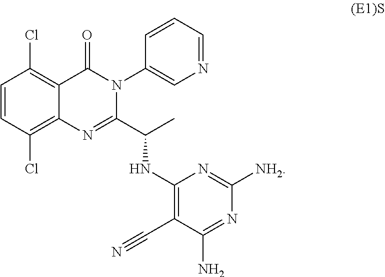 [SMILES: C[C@H](Nc1nc(N)nc(N)c1C#N)c1nc2c(Cl)ccc(Cl)c2c(=O)n1-c1cccnc1]

The (S) enantiomer of Compound E1 may also be referred to by its compound name: (S)-2,4-diamino-6-(1-(5,8-dichloro-4-oxo-3-(pyridin-3-yl)-3,4-dihydroquinazolin-2-yl)ethylamino) pyrimidine-5-carbonitrile using ChemDraw.

In some embodiments, the PI3Kδ inhibitor is Compound E2 having the structure:

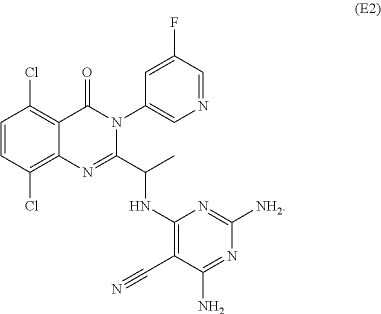 [SMILES: CC(Nc1nc(N)nc(N)c1C#N)c1nc2c(Cl)ccc(Cl)c2c(=O)n1-c1cncc(F)c1]

In some additional embodiments, Compound E2 is predominantly the S-enantiomer, having the structure:

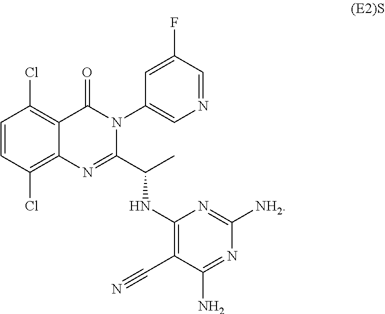 [SMILES: C[C@H](Nc1nc(N)nc(N)c1C#N)c1nc2c(Cl)ccc(Cl)c2c(=O)n1-c1cncc(F)c1]

The (S) enantiomer of Compound E2 may also be referred to by its compound name: (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile using ChemDraw.

In certain embodiments, the PI3Kδ inhibitor is Compound E3 having the structure:

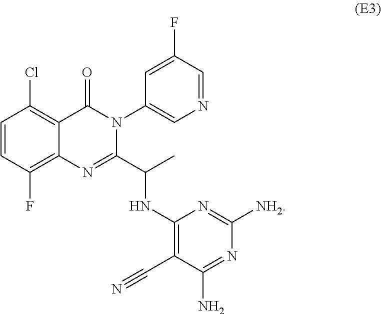 [SMILES: CC(Nc1nc(N)nc(N)c1C#N)c1nc2c(F)ccc(Cl)c2c(=O)n1-c1cncc(F)c1]

In certain additional embodiments, Compound E3 is predominantly the S-enantiomer, having the structure:

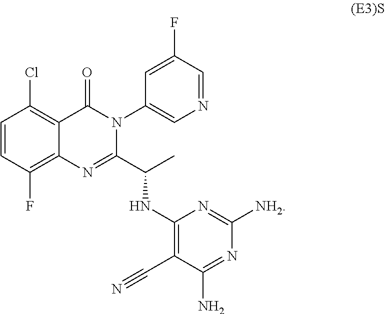 [SMILES: C[C@H](Nc1nc(N)nc(N)c1C#N)c1nc2c(F)ccc(Cl)c2c(=O)n1-c1cncc(F)c1]

The (S) enantiomer of Compound E3 may also be referred to by its compound name: (S)-2,4-diamino-6-(1-(5-chloro-8-fluoro-3-(5-fluoropyridin-3-yl)-4-oxo-3,4-dihydroquinazolin-2-yl)ethylamino)pyrimidine-5-carbonitrile using ChemDraw.

In some other embodiments, the PI3Kδ inhibitor is Compound E4 having the structure:

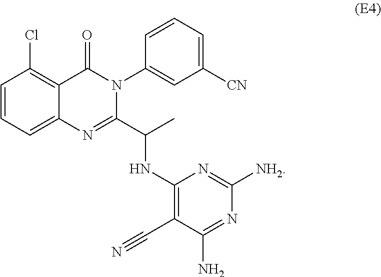 [SMILES: CC(Nc1nc(N)nc(N)c1C#N)c1nc2cccc(Cl)c2c(=O)n1-c1cccc(C#N)c1]

In additional embodiments, Compound E4 is predominantly the S-enantiomer, having the structure:

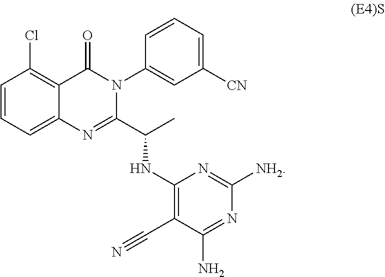 [SMILES: C[C@H](Nc1nc(N)nc(N)c1C#N)c1nc2cccc(Cl)c2c(=O)n1-c1cccc(C#N)c1]

The (S) enantiomer of Compound E4 may also be referred to by its compound name: (S)-2,4-diamino-6-((1-(5-chloro-3-(3-cyanophenyl)-4-oxo-3,4-dihydroquinazolin-2-yl)ethyl)amino) pyrimidine-5-carbonitrile using ChemDraw.

In certain other embodiments, the PI3Kδ inhibitor is Compound E5 having the structure:

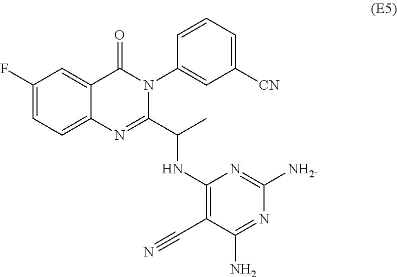 [SMILES: CC(Nc1nc(N)nc(N)c1C#N)c1nc2ccc(F)cc2c(=O)n1-c1cccc(C#N)c1]

In additional embodiments, Compound E5 is predominantly the S-enantiomer, having the structure:

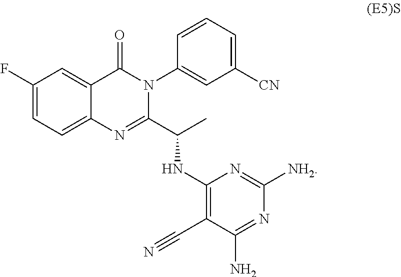 [SMILES: C[C@H](Nc1nc(N)nc(N)c1C#N)c1nc2ccc(F)cc2c(=O)n1-c1cccc(C#N)c1]

The (S) enantiomer of Compound E5 may also be referred to by its compound name: (S)-2,4-diamino-6-((1-(3-(3-cyanophenyl)-6-fluoro-4-oxo-3,4-dihydroquinazolin-2-yl)ethyl)amino) pyrimidine-5-carbonitrile using ChemDraw.

In yet other embodiments, the PI3Kδ inhibitor is Compound E6 having the structure:

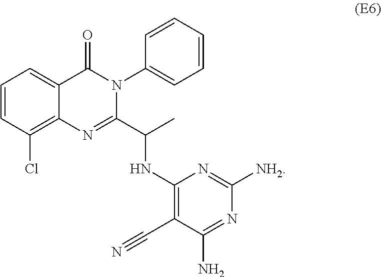 [SMILES: CC(Nc1nc(N)nc(N)c1C#N)c1nc2c(Cl)cccc2c(=O)n1-c1ccccc1]

In yet additional embodiments, Compound E6 is predominantly the S-enantiomer, having the structure:

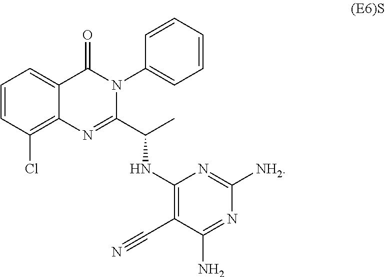 [SMILES: C[C@H](Nc1nc(N)nc(N)c1C#N)c1nc2c(Cl)cccc2c(=O)n1-c1ccccc1]

The (S) enantiomer of Compound E6 may also be referred to by its compound name: (S)-2,4-diamino-6-((1-(8-chloro-4-oxo-3-phenyl-3,4-dihydroquinazolin-2-yl)ethyl)amino)pyrimidine-5-carbonitrile using ChemDraw.

In other embodiments, the PI3Kδ inhibitor is Compound E7 having the structure:

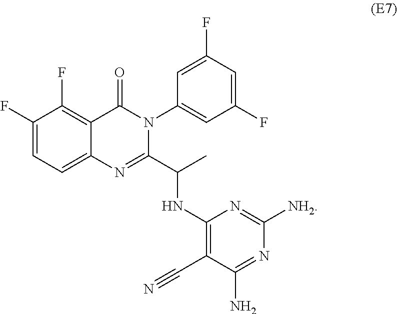 [SMILES: CC(Nc1nc(N)nc(N)c1C#N)c1nc2ccc(F)c(F)c2c(=O)n1-c1cc(F)cc(F)c1]

In other additional embodiments, Compound E7 is predominantly the S-enantiomer, having the structure:

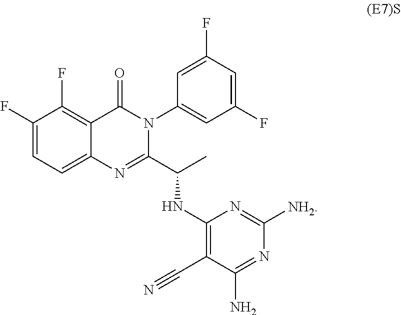 [SMILES: C[C@H](Nc1nc(N)nc(N)c1C#N)c1nc2ccc(F)c(F)c2c(=O)n1-c1cc(F)cc(F)c1]

The (S) enantiomer of Compound E7 may also be referred to by its compound name: (S)-2,4-diamino-6-((1-(3-(3,5-difluorophenyl)-5,6-difluoro-4-oxo-3,4-dihydroquinazolin-2-yl)ethyl)amino)pyrimidine-5-carbonitrile using ChemDraw.

In another embodiments, the PI3Kδ inhibitor is Compound E8 having the structure:

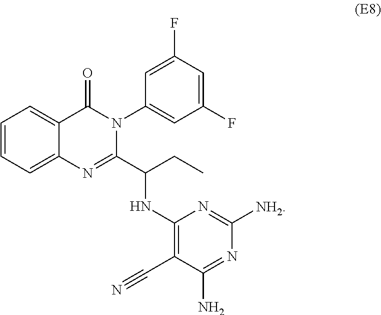 [SMILES: CCC(Nc1nc(N)nc(N)c1C#N)c1nc2ccccc2c(=O)n1-c1cc(F)cc(F)c1]

In additional embodiments, Compound E8 is predominantly the S-enantiomer, having the structure:

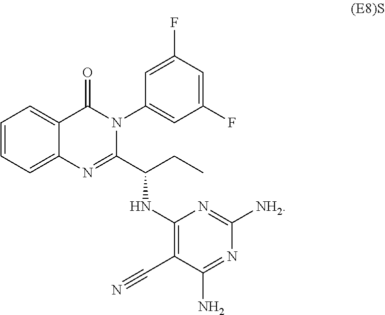 [SMILES: CC[C@H](Nc1nc(N)nc(N)c1C#N)c1nc2ccccc2c(=O)n1-c1cc(F)cc(F)c1]

The (S) enantiomer of Compound E8 may also be referred to by its compound name: (S)-2,4-diamino-6-((1-(3-(3,5-difluorophenyl)-4-oxo-3,4-dihydroquinazolin-2-yl)propyl)amino)pyrimidine-5-carbonitrile using ChemDraw.

In some other embodiments, the PI3Kδ inhibitor is Compound E9 having the structure:

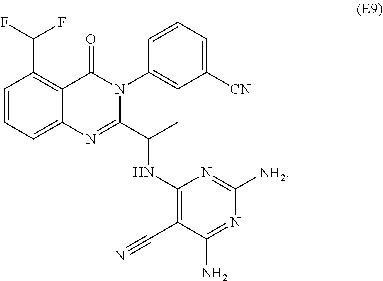 [SMILES: CC(Nc1nc(N)nc(N)c1C#N)c1nc2cccc(C(F)F)c2c(=O)n1-c1cccc(C#N)c1]

In additional embodiments, Compound E9 is predominantly the S-enantiomer, having the structure:

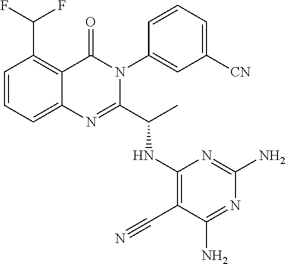 [SMILES: C[C@H](Nc1nc(N)nc(N)c1C#N)c1nc2cccc(C(F)F)c2c(=O)n1-c1cccc(C#N)c1]

The (S) enantiomer of Compound E9 may also be referred to by its compound name: (S)-2,4-diamino-6-((1-(3-(3-cyanophenyl)-5-(difluoromethyl)-4-oxo-3,4-dihydroquinazolin-2-yl)ethyl)amino)pyrimidine-5-carbonitrile using ChemDraw.

In yet other embodiment, the PI3K inhibitor is Compound E, whose (S)-enantiomer having the chemical name of (S)-2,4-diamino-6-((1-(3-(3,5-difluorophenyl)-5-fluoro-4-oxo-3,4-dihydroquinazolin-2-yl)ethyl)amino)pyrimidine-5-carbonitrile. The (S) enantiomer of Compound E has the structure:

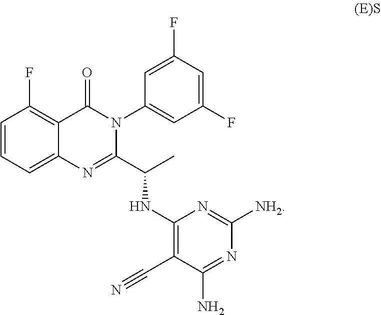 [SMILES: C[C@H](Nc1nc(N)nc(N)c1C#N)c1nc2cccc(F)c2c(=O)n1-c1cc(F)cc(F)c1]

In some other embodiment, the PI3K inhibitor is Compound E having the structure:

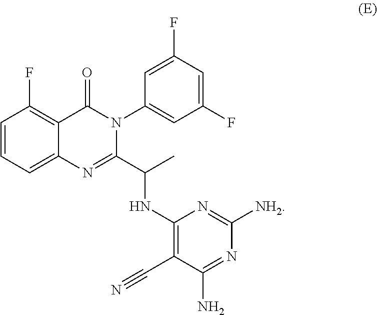 [SMILES: CC(Nc1nc(N)nc(N)c1C#N)c1nc2cccc(F)c2c(=O)n1-c1cc(F)cc(F)c1]

In additional embodiments, the PI3Kδ inhibitor is Compound F having the structure:

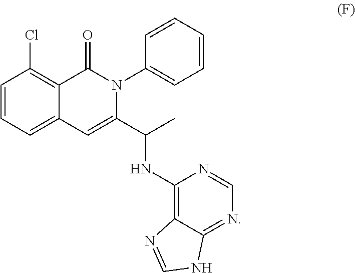 [SMILES: CC(Nc1ncnc2[nH]cnc12)c1cc2cccc(Cl)c2c(=O)n1-c1ccccc1]

In certain additional embodiments, Compound F is predominantly the S-enantiomer, having the structure:

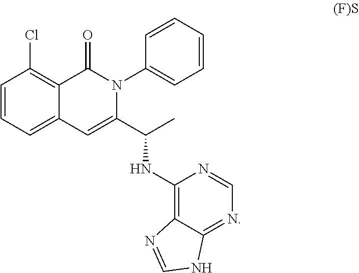 [SMILES: C[C@H](Nc1ncnc2[nH]cnc12)c1cc2cccc(Cl)c2c(=O)n1-c1ccccc1]

The (S) enantiomer of Compound F may also be referred to by its compound name: (S)-3-(1-((9H-purin-6-yl)amino)ethyl)-8-chloro-2-phenylisoquinolin-1(2H)-one using ChemDraw and may be synthesized as previously described in U.S. Pat. No. 8,193,182.

Compounds B, C, D, and E are PI3Kδ inhibitors, having selective inhibition of PI3K p110δ compared to other PI3K isoforms. Methods for synthesizing the compounds of formula II, Compounds B, C, D, and E are previously described in U.S. Pat. No. 7,932,260, U.S. Provisional Application Nos. 61/745,437 and 61/835,333. Further, Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, or Compound E9 are PI3Kδ inhibitors, having selective inhibition of PI3K p110δ compared to other PI3K isoforms, and may be synthesized as previously described in U.S. Provisional Application Nos. 61/745,437 and 61/835,333. The references are hereby incorporated herein by reference in their entirety.

Additional PI3K inhibitors include but are not limited to XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, AS252424, TGX221, TG100115, IC87114, and ZSTK474.

The SYK inhibitor includes but is not limited to 6-(1H-indazol-6-yl)-N-(4-morpholinophenyl)imidazo[1,2-a]pyrazin-8-amine, R406 (tamatinib), R788 (fostamatinib), PRT062607, BAY-61-3606, NVP-QAB 205 AA, R112, or R343, or a pharmaceutically acceptable salt thereof. See Kaur et al., European Journal of Medicinal Chemistry 67 (2013) 434-446. In one embodiment, the Syk inhibitor is 6-(1H-indazol-6-yl)-N-(4-morpholinophenyl)imidazo[1,2-a]pyrazin-8-amine as described in U.S. Pat. No. 8,450,321.

One skilled in the art understands that the compound structures may be named or identified using commonly recognized nomenclature systems and symbols. By way of example, the compound may be named or identified with common names, systematic or non-systematic names. The nomenclature systems and symbols that are commonly recognized in the art of chemistry include, for example, ChemBioDraw Ultra 12.0, Chemical Abstract Service (CAS) and International Union of Pure and Applied Chemistry (IUPAC). For example, the chemical name of Compound A may be referred to as N-(Cyanomethyl)-4-[2-(4-morpholinoanilino) pyrimidin-4-yl)benzamide using ChemDraw 2.0 or N-(cyanomethyl)-4-(2-((4-morpholinophenyl)amino)pyrimidin-4-yl)benzamide using IUPAC, and the chemical name of Compound B may be referred to as (S)-2-(1-((9H-purin-6-yl)amino)propyl)-5-fluoro-3-phenylquinazolin-4(3H)-one using ChemDraw 2.0 or (5-Fluoro-3-phenyl-2-R1S)-1-(9H-purin-6-ylamino)propyl]quinazolin-4(3H)-one) using IUPAC.

The term “selective inhibitor,” “selectively inhibits,” or variants refers to a compound or molecule that inhibits a member or isoform within the same protein family more effectively than at least one other member or isoform of the family. For example, the “PI3Kδ inhibitor” refers to a compound that inhibits the PI3Kδ isoform more effectively than at least one other isomers of the PI3K family, and the “JAK2 inhibitor” refers to a compound that inhibits JAK2 more effectively than at least one other members of the JAK family. The selective inhibitor may also be active against other members or isomers of the family, but requires higher concentrations to achieve the same degree of inhibition. “Selective” can also be used to describe a compound that inhibits a particular protein or kinase more so than a comparable compound.

The term “C₁₋₄alkyl” refers to straight chain or branched chain hydrocarbon groups having from 1 to 4 carbon atoms. Examples include methyl, ethyl, propyl, isopropyl, butyl, isobutyl, sec-butyl, and tert-butyl. Similarly, the term “C₁₋₆alkyl” refers to straight chain or branched chain hydrocarbon groups having from 1 to 6 carbon atoms

The term “halogen” refers to fluorine, chlorine, bromine and iodine.

The term “optionally substituted” refers to a group that is either unsubstituted or substituted with one or more groups selected from C₁₋₄ alkyl, C₃₋₆ cycloalkyl, C₂₋₆ alkenyl, C₂₋₆ alkynyl, C₁₋₆ alkylaryl, aryl, heterocycylyl, halo, haloC₁₋₆alkyl, haloC₃₋₆cycloalkyl, _{haloC2-6}alkenyl, haloC₂₋₆alkynyl, haloaryl, haloheterocycylyl, hydroxy, C₁₋₆ alkoxy, C₂₋₆alkenyloxy, C₂₋₆alkynyloxy, aryloxy, heterocyclyloxy, carboxy, haloC₁₋₆alkoxy, haloC₂₋₆alkenyloxy, haloC₂₋₆alkynyloxy, haloaryloxy, nitro, nitroC₁₋₆,alkyl, nitroC₂₋₆alkenyl, nitroaryl, nitroheterocyclyl, azido, amino, C₁₋₆alkylamino, C₂₋₆alkenylamino, C₂₋₆alkynylamino, arylamino, heterocyclamino acyl, C₁₋₆alkylacyl, C₂₋₆alkenylacyl, C₂₋₆alkynylacyl, arylacyl, heterocycylylacyl, acylamino, acyloxy, aldehydo, C₁₋₆alkylsulphonyl, arylsulphonyl, C₁₋₆alkylsulphonylamino, arylsulphonylamino, C₁₋₆alkylsulphonyloxy, arylsulphonyloxy, C₁₋₆alkylsulphenyl, C₂₋₆alklysulphenyl, arylsulphenyl, carboalkoxy, carboaryloxy, mercapto, C₁₋₆alkylthio, arylthio, acylthio, cyano and the like. Preferred substituents are selected from the group consisting of C₁₋₄ alkyl, C₃₋₆ cycloalkyl, C₂₋₆ alkenyl, C₂₋₆ alkynyl, C₁₋₆ alkylaryl, aryl, heterocycylyl, halo, haloaryl, haloheterocycylyl, hydroxy, C₁₋₄ alkoxy, aryloxy, carboxy, amino, C₁₋₆alkylacyl, arylacyl, heterocycylylacyl, acylamino, acyloxy, C₁₋₆alkylsulphenyl, arylsulphonyl and cyano.

The term “aryl” refers to single, polynuclear, conjugated or fused residues of aromatic hydrocarbons. Examples include phenyl, biphenyl, terphenyl, quaterphenyl, naphthyl, tetrahydronaphthyl, anthracenyl, dihydroanthracenyl, benzanthracenyl, dibenxanthracenyl and phenanthrenyl.

The term “unsaturated N-containing 5 or 6-membered heterocyclyl” refers to unsaturated, cyclic hydrocarbon groups containing at least one nitrogen. Suitable N-containing heterocyclic groups include unsaturated 5 to 6-membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl; unsaturated 5 or 6-membered heteromonocyclic group containing 1 to 2 oxygen atoms and 1 to 3 nitrogen atoms, such as, oxazolyl, isoxazolyl or oxadiazolyl; and unsaturated 5 or 6-membered heteromonocyclic group containing 1 to 2 sulphur atoms and 1 to 3 nitrogen atoms, such as, thiazolyl or thiadiazolyl.

The methods, compositions, kits and articles of manufacture provided herein use or include compounds (e.g., Compound A, Compound B, Compound C, Compound D, and Compound E) or pharmaceutically acceptable salts, prodrugs, or solvates thereof, in which from 1 to n hydrogen atoms attached to a carbon atom may be replaced by a deuterium atom or D, in which n is the number of hydrogen atoms in the molecule. In other embodiments, the methods, compositions, kits and articles of manufacture provided herein use or include Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, Compound E9 or pharmaceutically acceptable salts, prodrugs, or solvates thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of compounds or pharmaceutically acceptable salts, prodrugs, or solvates thereof, when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

As used herein, by “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” include, for example, salts with inorganic acids and salts with an organic acid. Examples of salts may include hydrochlorate, phosphate, diphosphate, hydrobromate, sulfate, sulfinate, nitrate, malate, maleate, fumarate, tartrate, succinate, citrate, acetate, lactate, mesylate, bismesylate, benzoate, salicylate, p-toluenesulfonate, 2-hydroxyethylsulfonate, stearate, and alkanoate (such as acetate, HOOC—(CH₂)ₙ—COOH where n is 0-4). In addition, the compounds described herein may be obtained as an acid addition salt, and the free base may be obtained by basifying a solution of the acid salt. Alternatively, the product may be a free base, an addition salt including a pharmaceutically acceptable addition salt may be produced by dissolving the free base in a suitable organic solvent and treating the solution with an acid, in accordance with commonly known procedures for preparing acid addition salts from base compounds. Those skilled in the art will recognize various synthetic methods that may be used to prepare nontoxic pharmaceutically acceptable addition salts. In one embodiment, Compound A is presented in a pharmaceutically acceptable hydrochloride salt. In other embodiment, ruxolitinib is presented in a pharmaceutically acceptable phosphate salt.

A “prodrug” includes any compound that becomes Compounds A, B, C, D, or E when administered to a subject, e.g., upon metabolic processing of the prodrug.

A “solvate” is formed by the interaction of a solvent and a compound. The compounds used in the methods and compositions (including, for example, pharmaceutical compositions, articles of manufacture and kits) may use or include solvates of salts of Compound A, Compound B, Compound C, Compound D, or Compound E. In some embodiment, the solvent may be hydrates of Compound F. In one embodiment, the solvent may be hydrates of Compound A, Compound B, Compound C, Compound D, or Compound E. In other embodiment, the solvent may be hydrates of Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, or Compound E9.

The methods, compositions, kits and articles of manufacture provided may use or include optical isomers, racemates, or other mixtures thereof, of Compound B, Compound C, Compound D, or Compound E or a pharmaceutically acceptable salt, prodrug, or solvate thereof. The single enantiomer or diastereomer, i.e., optically active form, may be obtained by asymmetric synthesis or by resolution of the racemate. Resolution of racemates may be accomplished, for example, by known methods such as crystallization in the presence of a resolving agent, or chromatography, using, for example a chiral high pressure liquid chromatography (HPLC) column. In addition, provided are also Z- and E-forms (or cis- and trans-forms) of Compounds B, C, D, or E, or a pharmaceutically acceptable salt, prodrug, or solvate thereof with carbon-carbon double bonds. The methods, compositions, kits and articles of manufacture provided may use or include any tautomeric form of Compounds B, C, D, or E, or a pharmaceutically acceptable salt, prodrug, or solvate thereof.

In some embodiments, the methods, compositions, kits and articles of manufacture provided herein may use or include a racemic mixture, or a mixture containing an enantiomeric excess (e.e.) of one enantiomer of Compound B, Compound C, Compound D, or Compound E. All such isomeric forms of Compounds B, C, D, or E are included herein the same as if each and every isomeric form were specifically and individually listed. For example, Compound B, Compound C, Compound D, or Compound E has an enantiomeric excess of at least 60%, at least 65%, at least 70%, at least 75%, at least 80%, at least 85%, at least 90%, at least 95%, at least 98%, or at least 99% of its (S)-enantiomer. In other examples, the methods, compositions, kits and articles of manufacture provided herein may use or include a racemic mixture, or a mixture containing an enantiomeric excess (e.e.) of one enantiomer of Compound F, which may be of at least 60%, at least 65%, at least 70%, at least 75%, at least 80%, at least 85%, at least 90%, at least 95%, at least 98%, or at least 99% of its (S)-enantiomer. In other embodiments, the methods, compositions, kits and articles of manufacture provided herein may use or include a racemic mixture, or a mixture containing an enantiomeric excess (e.e.) of one enantiomer of Compound D1, D2, D3, D4, D5, D6, D7, D8, D9, E1, E2, E3, E4, E5, E6, E7, E8, or E9. All such isomeric forms of Compounds D1-D9 or E1-E9 are included herein the same as if each and every isomeric form were specifically and individually listed. For example, Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, or Compound E9 has an enantiomeric excess of at least 60%, at least 65%, at least 70%, at least 75%, at least 80%, at least 85%, at least 90%, at least 95%, at least 98%, or at least 99% of its (S)-enantiomer.

By way of example, the methods, compositions, kits and articles of manufacture provided may use or include: (i) a mixture containing an enantiomeric excess of the (S)-enantiomer of Compound B, Compound C, Compound D, or Compound E or a pharmaceutically acceptable salt thereof; and (ii) Compound A, or ruxolitinib or a pharmaceutically acceptable salt thereof. Also, the methods, compositions, kits and articles of manufacture provided may use or include: (i) a mixture containing an enantiomeric excess of the (S)-enantiomer of Compound F or a pharmaceutically acceptable salt thereof; and (ii) Compound A, or ruxolitinib or a pharmaceutically acceptable salt thereof. In addition, the methods, compositions, kits and articles of manufacture provided may use or include: (i) a mixture containing an enantiomeric excess of the (S)-enantiomer of Compound D1, D2, D3, D4, D5, D6, D7, D8, D9, E1, E2, E3, E4, E5, E6, E7, E8, or E9 or a pharmaceutically acceptable salt thereof; and (ii) Compound A, or ruxolitinib or a pharmaceutically acceptable salt thereof. In some embodiments, the methods, compositions, kits and articles of manufacture provided herein use or include Compound B or a pharmaceutically acceptable salt thereof, in an enantiomeric excess of the (S)-enantiomer, and Compound A or a pharmaceutically acceptable salt thereof.

In some embodiment, the one or more therapeutic agents include inhibitors that are being used and/or developed to treat various hyperproliferative disorders such as cancer or myeloproliferative neoplasms. Exemplified therapeutic agents include compounds or molecules inhibiting pathways related to BCR, PI3K, SYK, and JAK, such as the agents inhibiting the RAS/RAFMEK/ERK pathway, the PI3K/PTEN/AKT/mTOR pathway, and the JAK-STAT pathway Inhibitors of mTOR include temsirolimus, everolimus, ridaforolimus (or deforolimus), OSI-027, AZD2014, CC-223, RAD001, LY294002, BEZ235, rapamycin, Ku-0063794, or PP242. Inhibitors of AKT include MK-2206, GDC-0068 and GSK795. Inhibitors of MEK includes trametinib, selumetinib, cobimetinib, MEK162, PD-325901, PD-035901, AZD6244, and CI-1040. The application also uses and includes other inhibitors, such as CDK inhibitors (AT-7519, SNS-032), JNK inhibitors (CC-401), MAPK inhibitors (VX-702, SB203580, SB202190), Raf inhibitors (PLX4720), ROCK inhibitor (Rho-15), Tie2 inhibitor (AMG-Tie2-1). As described herein, such inhibitors include compounds or agents that inhibit all subclasses (e.g. isoforms or members) of a target (e.g. PI3K alpha, beta, delta and gamma), compounds or agents that inhibit primarily one subclass, and compounds or agents that inhibit a subset of all subclasses.

In the present application, the one or more therapeutic agents, including the PI3K inhibitor and/or JAK inhibitor, may be used or combined with a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. In some embodiments, the one or more therapeutic agents are compounds or molecules that is an Abl inhibitor, an ACK inhibitor, an A2B inhibitor, an ASK inhibitor, an Aurora kinase inhibitor, a BTK inhibitor, a BRD inhibitor, a c-Kit inhibitor, a c-Met inhibitor, a CAK inhibitor, a CaMK inhibitor, a CDK inhibitor, a CK inhibitor, a DDR inhibitor, an EGFR inhibitor, a FAK inhibitor, a Flt-3 inhibitor, a FYN inhibitor, a GSK inhibitor, a HCK inhibitor, a HDAC inhibitor, an IKK inhibitor, an IDH inhibitor, an IKK inhibitor, a JAK inhibitor, a KDR inhibitor, a LCK inhibitor, a LOX inhibitor, a LOXL inhibitor, a LYN inhibitor, a MMP inhibitor, a MEK inhibitor, a MAPK inhibitor, a NEK9 inhibitor, a NPM-ALK inhibitor, a p38 kinase inhibitor, a PDGF inhibitor, a PI3 kinase (PI3K), a PK inhibitor, a PLK inhibitor, a PK inhibitor, a PYK inhibitor, a SYK inhibitor, a TPL2 inhibitor, a STK inhibitor, a STAT inhibitor, a SRC inhibitor, a TB K inhibitor, a TIE inhibitor, a TK inhibitor, a VEGF inhibitor, a YES inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

Chemotherapeutic agents may be categorized by their mechanism of action into, for example, the following groups: anti-metabolites/anti-cancer agents, such as pyrimidine analogs (floxuridine, capecitabine, and cytarabine); purine analogs, folate antagonists and related inhibitors antiproliferative/antimitotic agents including natural products such as vinca alkaloid (vinblastine, vincristine) and microtubule such as taxane (paclitaxel, docetaxel), vinblastin, nocodazole, epothilones and navelbine, epidipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prenisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) is meant to encompass any non-proteinaceous (i.e, non-peptidic) chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammaII and calicheamicin phiI1, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK®; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL®), Bristol Meyers Squibb Oncology, Princeton, N.J.) and docetaxel (TAXOTERE®), Rhone-Poulenc Rorer, Antony, France); chlorambucil; gemcitabine (Gemzar®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine®); novantrone; teniposide; edatrexate; daunomycin; aminopterin; xeoloda; ibandronate; CPT-11; topoisomerase inhibitor RFS 2000; difluoromethylomithine (DMFO); retinoids such as retinoic acid; capecitabine; FOLFIRI (fluorouracil, leucovorin, and irinotecan) and pharmaceutically acceptable salts, acids or derivatives of any of the above.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston®); inhibitors of the enzyme aromatase, which regulates estrogen production in the adrenal glands, such as, for example, 4(5)-imidazoles, aminoglutethimide, megestrol acetate (Megace®), exemestane, formestane, fadrozole, vorozole (Rivisor®), letrozole (Femara®), and anastrozole (Arimidex®); and anti-androgens such as flutamide, nilutamide, bicalutamide, leuprohde, and goserelin; and pharmaceutically acceptable salts, acids or derivatives of any of the above.

The anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproternase-2, plasminogen activator inhibitor-1, plasminogen activator inbibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydroproline, thiaproline, .alpha.-dipyridyl, beta-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpba-2-antiplasmin, bisantrene, lobenzarit disodium, n-2-carboxyphenyl-4-chloroanthronilic acid disodium or “CCA”, thalidomide; angiostatic steroid, cargboxynaminolmidazole; metalloproteinase inhibitors such as BB94. Other anti-angiogenesis agents include antibodies, preferably monoclonal antibodies against these angiogenic growth factors: beta-FGF, alpha-FGF, FGF-5, VEGF isoforms, VEGF-C, HGF/SF and Ang-1/Ang-2. See Ferrara N. and Alitalo, K. “Clinical application of angiogenic growth factors and their inhibitors” (1999) Nature Medicine 5:1359-1364.

The anti-fibrotic agents include, but are not limited to, the compounds such as beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. Also, the anti-fibrotic agents are copper chelating agents, penetrating or not penetrating the cells. Exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate.

The immunotherapeutic agents include and are not limited to therapeutic antibodies suitable for treating patients; such as abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8. The exemplified therapeutic antibodies may be further labeled or combined with a radioisotope particle, such as indium In 111, yttrium Y 90, iodine I-131.

In one embodiment, the one or more additional therapeutic agent may be an inhibitor to PI3K such as PI3Kγ, PI3Kδ, PI3Kβ, and PI3Kα, JAK such as JAK1 and JAK2, SYK, BTK, BRD such as BRD4, lysyl oxidase protein, lysyl oxidase-like protein (LOXL) such as LOXL1, LOXL2, LOXL3, LOXL4, or LOXL5, matrix metalloprotease (MMP) such as MMP 1-10, adenosine A2B receptor (A2b), isocitrate dehydrogenase (IDH) such as IDH1, apoptosis signal-regulating kinase (ASK) such as ASK1, serine/threonine kinase TPL2, discoidin domain receptor (DDR) such as DDR1 and DDR2, histone deacetylase (HDAC) inhibitor protein kinase C (PKC) or any combination thereof. In other embodiment, the one or more therapeutic agent may be a PI3K (including PI3Kγ, PI3Kδ, PI3Kβ, PI3Kα, and/or pan-PI3K) inhibitor, a JAK (including JAK1 and/or JAK2) inhibitor, a SYK inhibitor, a BTK inhibitor, an A2B (adenosine A2B receptor) inhibitor, an ACK (activated CDC kinase, including ACK1) inhibitor, an ASK (apoptosis signal-regulating kinase, including ASK1) inhibitor, Auroa kinase, a BRD (bromodomain-containing protein, including BRD4) inhibitor, a CAK (CDK-activating kinase) inhibitor, a CaMK (calmodulin-dependent protein kinases) inhibitor, a CDK (cyclin-dependent kinases, including CDK1, 2, 3, 4, and/or 6) inhibitor, a CK (casein kinase, including CK1 and/or CK2) inhibitor, a DDR (discoidin domain receptor, including DDR1 and/or DDR2) inhibitor, a EGFR inhibitor, a FAK (focal adhesion kinase) inhibitor, a GSK (glycogen synthase kinase) inhibitor, a HDAC (histone deacetylase) inhibitor, an IDH (isocitrate dehydrogenase, including IDH1) inhibitor, an IKK inhibitor, a LCK (lymphocyte-specific protein tyrosine kinase) inhibitor, a LOX (lysyl oxidase) inhibitor, a LOXL (lysyl oxidase like protein, including LOXL1, LOXL2, LOXL3, LOXL4, and/or LOXL5) inhibitor, a MEK inhibitor, a matrix metalloprotease (MMP, including MMP2 and/or MMP9) inhibitor, a mitogen-activated protein kinases (MAPK) inhibitor, a PDGF (platelet-derived growth factor) inhibitor, a phosphorylase kinase (PK) inhibitor, a PLK (polo-like kinase, including PLK1, 2, 3) inhibitor, a protein kinase (PK, including protein kinase A, B, C) inhibitor, a serine/threonine kinase (STK) inhibitor, a STAT (signal transduction and transcription) inhibitor, a TBK (serine/threonine-protein kinase, including TBK1) inhibitor, a TK (tyrosine kinase) inhibitor, a TPL2 (serine/threonine kinase) inhibitor, a NEK9 inhibitor, an Abl inhibitor, a p38 kinase inhibitor, a PYK inhibitor, a PYK inhibitor, a c-Kit inhibitor, a NPM-ALK inhibitor, a Flt-3 inhibitor, a c-Met inhibitor, a KDR inhibitor, a TIE-2 inhibitor, a VEGFR inhibitor, a SRC inhibitor, a HCK inhibitor, a LYN inhibitor, a FYN inhibitor, a YES inhibitor, or any combination thereof.

In other embodiments, the one or more therapeutic agent is: a JAK inhibitor, including but not limited to Compound A, ruxolitinib, fedratinib, tofacitinib, baricitinib, lestaurtinib, pacritinib, XL019, AZD1480, INCB039110, LY2784544, BMS911543, and NS018; a myelofibrosis inhibiting agent, including but not limited to, hedgehog inhibitors (saridegib), histone deacetylase (HDAC) inhibitors (pracinostat, panobinostat), tyrosine kinase inhibitor (lestaurtinib); a discoidin domain receptor (DDR) inhibitor, including but not limited to, those disclosed in US2009/0142345, US2011/0287011, WO2013/027802, WO2013/034933, and U.S. Provisional Application No. 61/705,044; a MMP9 inhibitor, including but not limited to, marimastat (BB-2516), cipemastat (Ro 32-3555), and those described in WO2012/027721; a LOXL inhibitor, including but not limited to the antibodies described in WO2009/017833; a LOXL2 inhibitor, including but not limited to the antibodies described in WO2009/017833, WO2009/035791 and WO/2011/097513; an ASK1 inhibitor, including but not limited to, those described in WO2011/008709 and WO/2013/112741; a PI3Kδ inhibitor, including but not limited to, Compound B, Compound C, Compound D, Compound E, the compounds described in U.S. Pat. No. 7,932,260, U.S. Provisional Application Nos. 61/745,437 and 61/835,333, PI3K II, TGR-1202, AMG-319, GSK2269557, X-339, X-414, RP5090, KAR4141, XL499, OXY111A, IPI-145, IPI-443; a PI3Kβ inhibitor, including but not limited to, GSK2636771, BAY 10824391, TGX221; a PI3Kα inhibitor, including but not limited to, Buparlisib, BAY 80-6946, BYL719, PX-866, RG7604, MLN1117, WX-037, AEZS-129, PA799; a PI3Kγ inhibitor, including but not limited to, ZSTK474, AS252424, LY294002, TG100115; a pan PI3K inhibitor, including but not limited to, LY294002, BEZ235, XL147 (SAR245408), GDC-0941; additional PI3K inhibitors, including but not limited to BKM120, CH5132799, XL756, and GDC-0980, wortmannin; a BTK inhibitor, including but not limited to, ibrutinib, HM71224, ONO-4059, CC-292; a SYK inhibitor, including but not limited to, tamatinib (R406), fostamatinib (R788), PRT062607, BAY-61-3606, NVP-QAB 205 AA, R112, R343, or those described in U.S. Pat. No. 8,450,321; a BRD4 inhibitor, an IDH1 inhibitor, a TPL2 inhibitor, an A2b inhibitor, or agents that activate or reactivate latent human immunodeficiency virus (HIV), or a protein kinase C (PKC) activator, romidepsin or panobinostat. In another embodiment, JAK inhibitors include, but not limited to, Decemotinib (or VX-509), GLPG0634, or GLPG0788, or a pharmaceutically acceptable salt thereof.

In certain embodiments, the methods, compositions, kits, and articles of manufacture for treating MPN that use or include Compound A or a pharmaceutically acceptable salt thereof or ruxolitinib or a pharmaceutically acceptable salt thereof as the JAK inhibitor; and Compound B or a pharmaceutically acceptable salt thereof, Compound C or a pharmaceutically acceptable salt thereof, Compound D or a pharmaceutically acceptable salt thereof, or Compound E or a pharmaceutically acceptable salt thereof as the PI3Kδ inhibitor. In other embodiments, the JAK inhibitor is Compound A or a pharmaceutically acceptable salt thereof. In another embodiment, the JAK inhibitor is ruxolitinib or a pharmaceutically acceptable salt thereof. In additional embodiments, the PI3K inhibitor is Compound B or a pharmaceutically acceptable salt thereof. In other embodiments, the PI3K inhibitor is Compound C or a pharmaceutically acceptable salt thereof. In some other embodiments, the PI3K inhibitor is Compound D or a pharmaceutically acceptable salt thereof. In yet another embodiment, the PI3K compound is Compound E or a pharmaceutically acceptable salt thereof. In other embodiments, the PI3K inhibitor is Compound F or a pharmaceutically acceptable salt thereof. In some embodiments, the methods, compositions, kits, and articles of manufacture for treating MPN that use or include Compound A or a pharmaceutically acceptable salt thereof or ruxolitinib or a pharmaceutically acceptable salt thereof as the JAK inhibitor; and Compound F or a pharmaceutically acceptable salt thereof, Compound D1-D9 or a pharmaceutically acceptable salt thereof, or Compound E1-E9 or a pharmaceutically acceptable salt thereof as the PI3Kδ inhibitor. In yet other embodiments, the PI3K compound is Compound F, Compound D1, Compound D2, Compound D3, Compound D4, Compound D5, Compound D6, Compound D7, Compound D8, or Compound D9, or a pharmaceutically acceptable salt thereof. In certain other embodiments, the PI3K compound is Compound E1, Compound E2, Compound E3, Compound E4, Compound E5, Compound E6, Compound E7, Compound E8, or Compound E9, or a pharmaceutically acceptable salt thereof.

### Methods for Treatment

The present application provides methods for treating hyperproliferative diseases in a subject (e.g., a human) comprising administering to the subject (e.g., a human) a therapeutically effective amount of one or more of inhibitors, including a PI3K inhibitor, a JAK inhibitor, a SYK inhibitor, a BTK inhibitor, and a BRD inhibitor. The present application also provides a therapeutically effective amount of one or more inhibitors, including a PI3K inhibitor, a JAK inhibitor, a SYK inhibitor, a BTK inhibitor, and a BRD inhibitor for use in a method for treating hyperproliferative diseases in a subject (e.g., a human) comprising administering to the subject (e.g., a human) said one or more inhibitors. In one embodiment, the method comprises administering to the subject (i.e. a human) a therapeutically effective amount of a JAK inhibitor, including a JAK2 inhibitor. In another embodiment, the method comprises administering to the subject (i.e. a human) a therapeutically effective amount of a PI3K inhibitor, including a PI3Kδ inhibitor. In additional embodiment, the method comprises administering to the subject (i.e. a human) a therapeutically effective amount of a JAK inhibitor, a therapeutically effective amount of a PI3K inhibitor, and a therapeutically effective amount of additional therapeutic agent. In certain embodiments, the method comprises a therapeutically effective amount of a JAK inhibitor and a therapeutically effectively amount of a PI3Kδ inhibitor. In some embodiments, the method comprises administering to a human a therapeutically effective amount of Compound A or ruxolotinib, or a pharmaceutically acceptable salt thereof, and a therapeutically effective amount of Compound B, Compound C, Compound D, or Compound E, or a pharmaceutically acceptable salt thereof. In one embodiment, the method comprises administering to a human a therapeutically effective amount of Compound A or a pharmaceutically acceptable salt thereof, and a therapeutically effective amount of Compound B, C, D, or E. In another embodiment, the method comprises administering to a human a therapeutically effective amount of Compound A or a pharmaceutically acceptable salt thereof, and a therapeutically effective amount of Compound B or a pharmaceutically acceptable salt thereof. In other embodiment, the method comprises administering to a human a therapeutically effective amount of ruxolitinib or a pharmaceutically acceptable salt thereof, and a therapeutically effective amount of Compound B, C, D, or E. In yet another embodiment, the method comprises administering to a human a therapeutically effective amount of ruxolotinib or a pharmaceutically acceptable salt thereof, and a therapeutically effective amount of Compound B or a pharmaceutically acceptable salt thereof. In some embodiment, the method comprises administering to a human a therapeutically effective amount of Compound A or a pharmaceutically acceptable salt thereof, and a therapeutically effective amount of Compound F or a pharmaceutically acceptable salt thereof. In some other embodiment, the method comprises administering to a human a therapeutically effective amount of Compound A or a pharmaceutically acceptable salt thereof, and a therapeutically effective amount of Compound F or a pharmaceutically acceptable salt thereof. In certain other embodiment, the method comprises administering to a human a therapeutically effective amount of ruxolitinib or a pharmaceutically acceptable salt thereof, and a therapeutically effective amount of Compound F or a pharmaceutically acceptable salt thereof. In one embodiment, the method comprises administering to a human a therapeutically effective amount of Compound A or a pharmaceutically acceptable salt thereof, and a therapeutically effective amount of Compound D1, Compound D2, Compound D3, Compound D4, Compound D5, Compound D6, Compound D7, Compound D8, or Compound D9 or a pharmaceutically acceptable salt thereof. In other embodiment, the method comprises administering to a human a therapeutically effective amount of Compound A or a pharmaceutically acceptable salt thereof, and a therapeutically effective amount of Compound E1, Compound E2, Compound E3, Compound E4, Compound E5, Compound E6, Compound E7, Compound E8, or Compound E9, or a pharmaceutically acceptable salt thereof.

The patients may have or have not received prior drug therapy. In one embodiment, the method provides a treatment or therapeutic to hyperproliferative disease patients who have been treated or are currently being treated with thalidomide or with a derivative thereof, such as lenalidomide, or other JAK inhibitor such as ruxolotinib or TG101348. In certain embodiments, the method comprises treating patients who have received prior drug treatment using a JAK inhibitor.

In some embodiments, the method comprises treating patients who have received prior drug treatment using a JAK inhibitor over a period of time (i.e. chronic JAK therapy) and developed disease persistence. Patients who have received chronic ruxolitinib (i.e. over 3-6 months, more than 6 months, or more than one year) commonly develop disease persistence. As used herein, disease persistence refers to patients showing gradual return of splenomegaly and/or constitutional symptoms, the lack of hematologic or molecular remissions, or the loss of clinical improvement.

The hyperproliferative disease includes cancer and myeloproliferative disease such as cellular-proliferative disease in cardiac, lung, gastrointestine, genitourinary tract, liver, bone, nerve system, gynecological, hematological, skin, and adrenal glands.

Myeloproliferative Disease

Myeloproliferative diseases (MPD) or myeloproliferative neoplasms (MPN) are a diverse group of clonal disorders of pluripotent hematopoietic stem cells that have increase or overproduction of one or more myeloid cells, growth factor independent colony formation in vitro, marrow hypercellularity, extramedullary hematopoiesis, splenomegaly, hepatomegaly, and thrombotic and/or hemorrhagic diathesis. The myleoproliferative diseases or neoplasms include, but are not limited to, polycythemia vera (PV), primary myelofibrosis (PMF), thrombocythemia, essential thrombocythemia (ET), agnoneic myeloid metaplasia (AMM), idiopathic myelofibrosis (IMF), chronic myelogenous leukemia (CML), systemic mastocystosis (SM), chronic neutrophilic leukemia (CNL), myelodisplastic syndrome (MDS), and systemic mast cell disease (SMCD). In some embodiments, the myloproliferative disease is polycythemia vera (PV), essential thrombocythemia (ET), and primary myelofibrosis (PMF). In certain embodiments, the myloproliferative disease is polycythemia vera (PV). In other embodiment, the myeloproliferative disease is essential thrombocythemia (ET). In another embodiment, the myeloproliferative disease is primary myelofibrosis (PMF).

The chronic myeloproliferative neoplasms (MPNs) are acquired marrow disorders characterized by excessive production of mature myeloid cells. Major morbidity from these conditions result from thrombo-hemorrhagic complications (arterial and venous thrombosis, major bleeding) and transformation to acute leukemia such as acute myeloid leukemia (AML). Myelofibrosis originates from acquired mutations that alter the hematopoietic stem cell and produce alterations in the kinase-mediated signaling processes, resulting in clonal myeloproliferation, bone marrow fibrosis, and abnormal cytokine expression (Tefferi et al). PMF is a rare disease with an incidence of 0.4 to 1.3 per 100,000 people in Europe, Australia, and U.S. Myelofibrosis can also occur in patients with PV (10-20% of subjects after 10-20 years) and ET (2-3% of subjects), in which case it is called post-ET/PV MF. The pathogenic mechanism in PMF may be the unchecked proliferation of a hematopoietic stem cell clone that leads to ineffective erythropoiesis, atypical megakaryocytic hyperplasia, and an increase in the ratio of immature granulocytes to total granulocytes. The clonal myeloproliferation is characteristically accompanied by bone marrow fibrosis and extramedullary hematopoiesis in the spleen, liver, and other organs. Other features of extramedullary hematopoiesis on a blood smear include teardrop-shaped red cells, nucleated red cells, and myeloid immaturity. Additional clinical features include marked splenomegaly, progressive anemia, and constitutional symptoms.

An international working group (IWG) for myeloproliferative neoplasms research and treatment (IWG-MRT) has defined myeloproliferative diseases and related conditions (Vannucchi et al., CA Cancer J. Clin., 59:171-191, 2009) that are used in the present application. Patients, who present with MPN or PMF, are identifiable in the art using the IWG-MRT criteria. Subjects “at risk for” certain MPN are subjects having an early stage form of the disease, and may for instance include subjects having a genetic marker thereof, such as the JAK2V617F allele which is associated with PV (>95%), with ET (60%) and with PMF (60%). In addition, subjects are considered to be “at risk for” certain MPN if they already manifest symptoms of an earlier stage form. For example, subjects presenting with MPN are at risk for post-PV and post-ET, both of which develop following MPN.

Compound A is a JAK inhibitor and provides improved clinical response in MPN patients, including PMF. One of the improved outcomes is improvement in anemia response and/or in spleen response. By “anemia response” is meant an increase in the patient's hemoglobin level or a patient who was transfusion dependent becoming transfusion independent. Desirably, a minimum increase in hemoglobin of 2.0 g/dL lasting a minimum of 8 weeks is achieved, which is the level of improvement specified in the International Working Group (IWG) consensus criteria. However, smaller, but still medically significant, increases in hemoglobin are also considered to be within the term “anemia response”. By “spleen response” is meant a reduction in the size of the patient's spleen as assessed by either palpation of a previously palpable spleen during physical exam or by diagnostic imaging. The IWG consensus criteria specifies that there be either a minimum 50% reduction in palpable splenomegaly (spleen enlargement) of a spleen that is at least 10 cm at baseline (prior to treatment) or of a spleen that is palpable at more than 5 cm at baseline becomes not palpable. However, smaller reductions are also considered to be within the term “spleen response”.

One aspect of the present application provides the methods, composition, and kit for the patient who has received prior drug therapy or is current in drug therapy. By way of example, the patients have been treated, or are currently being treated, with thalidomide, lenalidomide, pomalidomide or derivative thereof, that are used in the treatment of multiple myeloma, and appear also to be showing some benefit in patients afflicted with myeloproliferative disorder. In another example, the patients have been treated, or are undergoing treatment, with a JAK inhibitor other than Compound A, including but not limited to INCB018424, TG101348, ruxolitinib. Patients will either be undergoing treatment with the other JAK2 inhibitor or will have been treated with such a drug within a time frame, relative to the composition or treatment provided herein, sufficient for the effects of that JAK2 inhibitor to be manifest in the patient. In general, INCB018424 is administered at starting doses of 15 or 20 mg BID with dose titration from 5 mg BID to 25 mg BID; TG101348 is administered once a day with a maximum tolerated dose (MTD) determined to be 680 mg/day; and ruxolitinib is administered at a stable dose of 20, 15, or 5 mg (based on platelet count) BID.

In certain embodiment, the MPD patients have not received any drug treatment, i.e. naïve. The naïve MPD patients may subsequently receive treatment or therapeutic described herein. For example, the naïve MPD patients may receive a PI3K inhibitor, a JAK inhibitor, additional therapeutic agent, or any combination thereof.

Patients receive the treatment or composition according to the present application experience an improved response when they are selected initially based on an elevation in the level of any one or more of the markers noted above. An elevated level is a level that is greater than the level in a normal subject. As used herein, the “level” of a given marker is considered to be altered, i.e., either elevated or reduced, when the level measured in a given patient is different to a statistically significant extent from the corresponding level in a normal subject. Patients that present with marker levels altered to an extent sufficient, desirably, to yield a p value of at least 0.05 or more significant, i.e., better, are suitable candidate for the therapy described herein. In embodiments, the p value is at least 0.03, 0.02 or 0.01, and in preferred embodiments the p value is at least 0.009, 0.007, 0.005, 0.003, 0.001 or better. The levels of a given marker can be determined using assays already well established for detection the markers noted above. In embodiments, this is achieved by extracting a biological sample from the patient candidate, such as a sample of whole blood or a fraction thereof such as plasma or serum. The sample then is treated to enrich for the marker of interest, if desired, and the enriched or neat sample is assayed for instance using a detectable ligand for the marker, such as a labeled antibody that binds selectively to the marker. The amount of marker present in the sample can then be determined either semi-quantitatively or quantitatively, to obtain a value that is then compared against a reference value that is the normal level for that marker in a healthy subject. As noted above, a difference in marker levels sufficient to arrive at a p value that is at least 0.05 indicates an altered marker level of significance, and patients presenting with an elevated level of that marker (or in the case of eotaxin, a decreased level) are candidates to be treated using the method, composition, kit of the present application.

Also suitable as candidates for the therapy are those patients that meet certain clinical criteria, including those presenting with a spleen of relatively small size, and those presenting with an elevated level of circulating, or peripheral, blasts. In one embodiment, the selected patient is one that has not yet progressed to transfusion dependency. Splenic enlargement is assessed by palpation. Splenic size and volume can also be measured by diagnostic imaging such as ultrasound, CT or MRI). Normal spleen size is approximately 11.0 cm. in craniocaudal length.

Also suitable as candidates for the therapy are those patients presenting with a lower percentage of circulating blasts. Blasts are immature precursor cells that are normally found in the bone marrow and not the peripheral blood. They normally give rise to mature blood cells. The lower percentage of circulating blasts is measured by cytomorphologic analysis of a peripheral blood smear as well as multiparameter flow cytometry and immunohistochemistry. As a prognostic factor >1=1% blasts is used.

In another aspect, the application provides the methods, composition, and kits for the patients who have received prior therapy and exhibit suboptimal response. The suboptimal response to prior drug therapy may be characterized by ineffective erythropoiesis and bone marrow fibrosis with extramedullary hematopoiesis manifested by marked hepatosplenomegaly due in part to the emergence of a clone of cells that are non-responsive or resistant to the prior drug therapy. It has been shown that patients receive ruxolitinib develop resistance or non-response after a period of time. Such disease may be observed after 3, 4, 5, 6, 7, 8, 9, 10, 11, or 12 months or years of ruxolitinb treatment.

The biologic mechanism for suboptimal responses is unclear. Although resistance mutations within JAK2 have not been identified as a basis for acquired resistance to JAK inhibitors, heterodimeric JAK-STAT activation is a potential mechanism of disease persistence. JAK inhibitor persistent cells may develop through exposure to JAK inhibitors, and such cells may exhibit lower apoptosis in response to ongoing exposure these drugs. This may cause reactivation of JAK2 phosphorylation and the downstream STAT3, STAT5, and MAP kinase signaling in persistent cells which would no longer be inhibited by JAK inhibitors. JAK family members JAK1 and TYK2 associate with JAK2 in persistent cells, resulting in re-activation of JAK2.

The persistence phenomenon is reversible, and cells become re-sensitized or responsive with withdrawal of the JAK inhibitor. These re-sensitized cells suggest a loss of the association between JAK1/TYK2 and JAK2, resulting in loss of JAK2 activation. This phenomenon of JAK inhibitor persistence is observed in vivo in MPN murine models, and in primary samples of patients treated with the ruxolitinib.

The present application shows that the PI3Kδ isoform was expressed and the prominent isoform (i.e. highest expression levels) among PI3K isoforms α, β, δ, and γ in progenitor cells from MF patients. In addition, the present application showed that PI3Kδ inhibitors inhibited basal (TPO-untreated) and thrombopoietin (TPO)-treated AKT/S6RP phosphorylation (p-AKT/p-S6RP) in PBMC from MF patients. MF patients were either on chronic ruxolitinib therapy or had not received ruxolitinib or other JAK inhibitors (i.e. naïve). It is hypothesized that, upon activation of the MPL receptor by thrombopoietin (TPO), JAK2 is recruited to the membrane which activates downstream signaling pathways including STAT3/5, PI3K and RAS, resulting in increased proliferation, survival, metabolism and cellular motility. About 50-60% of primary MF patients harbor the activating JAK2V617F mutation which constitutively activates the signaling cascade.

According to the present application, the combination of a PI3Kδ inhibitor and a JAK inhibitor results in enhanced therapeutic responses (including beneficial or synergistic effects). Also, concurrent targeting of PI3K and JAK/STAT pathway may represent a new therapeutic treatment to optimize efficacy and reduce toxicity in patients with MPN.

Cancers

The methods described herein may be used to treat various types of cancers. In some embodiments, the cancer may be a hematological malignancy, including relapsed or refractory hematologic malignancies. Cancers amenable to treatment using the methods described herein may include leukemias, lymphomas, and multiple myeloma. Leukemias may include, for example, lymphocytic leukemias and chronic myeloid (myelogenous) leukemias. Lymphomas may include, for example, malignant neoplasms of lymphoid and reticuloendothelial tissues, such as Burkitt's lymphoma, Hodgkin's lymphoma, non-Hodgkin's lymphomas (including, for example, indolent non-Hodgkin's lymphoma), and lymphocytic lymphomas.

In some embodiments, the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL) In one embodiment, the cancer is minimal residual disease (MRD). In one embodiment, the cancer is DLBCL, including activated B-cell (ABC)-DLBCL and a germinal center B-cell (GCB)-like DLBCL.

In certain embodiments, cancer is a solid tumor is selected from the group consisting of pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma. In some embodiment, the cancer is pancreatic cancer.

Any of the methods of treatment provided may be used to treat cancer at various stage. By way of example, the cancer stage includes but is not limited to early, advanced, locally advanced, remission, refactory, reoccurred after remission and progressive. As described in the present application, concurrent targeting of PI3K/AKT and JAK/STAT pathways (by simultaneous or sequential administration) may provide a new therapeutic treatment to optimize patient response and/or reduce resistance or relapse from targeting either PI3K/AKT or JAK/STAT pathways alone.

Subjects

Any of the methods of treatment provided may be used to treat a subject (e.g., human) who has been diagnosed with or is suspected of having cancer. As used herein, a subject refers to a mammal, including, for example, a human.

In some embodiments, the subject may be a human who exhibits one or more symptoms associated with cancer or hyperproliferative disease. In certain, the subject may be a human who is at risk, or genetically or otherwise predisposed (e.g., risk factor) to developing cancer or hyperproliferative disease who has or has not been diagnosed. As used herein, an “at risk” subject is a subject who is at risk of developing cancer. The subject may or may not have detectable disease, and may or may not have displayed detectable disease prior to the treatment methods described herein. An at risk subject may have one or more so-called risk factors, which are measurable parameters that correlate with development of cancer, which are described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be a human who is undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In certain embodiments, the subject is refractory to at least one, at least two, at least three, or at least four chemotherapy treatment (including standard or experimental chemotherapy) selected from fludarabine, rituximab, obinutuzumab, alkylating agents, alemtuzumab and other chemotherapy treatments such as CHOP (cyclophosphamide, doxorubicin, vincristine, prednisone); R-CHOP (rituximab-CHOP); hyperCVAD (hyperfractionated cyclophosphamide, vincristine, doxorubicin, dexamethasone, methotrexate, cytarabine); R-hyperCVAD (rituximab-hyperCVAD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and Velcade®; Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D. T. PACE (dexamethasone, thalidomide, cisplatin, Adriamycin®, cyclophosphamide, etoposide).

Other examples of chemotherapy treatments (including standard or experimental chemotherapies) are described below. In addition, treatment of certain lymphomas is reviewed in Cheson, B. D., Leonard, J. P., “Monoclonal Antibody Therapy for B-Cell Non-Hodgkin's Lymphoma” The New England Journal of Medicine 2008, 359(6), p. 613-626; and Wierda, W. G., “Current and Investigational Therapies for Patients with CLL” Hematology 2006, p. 285-294. Lymphoma incidence patterns in the United States is profiled in Morton, L. M., et al. “Lymphoma Incidence Patterns by WHO Subtype in the United States, 1992-2001” Blood 2006, 107(1), p. 265-276.

Examples of immunotherapeutic agents treating lymphoma or leukemia include, but are not limited to, rituximab (such as Rituxan), alemtuzumab (such as Campath, MabCampath), anti-CD19 antibodies, anti-CD20 antibodies, anti-MN-14 antibodies, anti-TRAIL, Anti-TRAIL DR4 and DR5 antibodies, anti-CD74 antibodies, apolizumab, bevacizumab, CHIR-12.12, epratuzumab (hLL2-anti-CD22 humanized antibody), galiximab, ha20, ibritumomab tiuxetan, lumiliximab, milatuzumab, ofatumumab, PRO131921, SGN-40, WT-1 analog peptide vaccine, WT1 126-134 peptide vaccine, tositumomab, autologous human tumor-derived HSPPC-96, and veltuzumab. Additional immunotherapy agents includes using cancer vaccines based upon the genetic makeup of an individual patient's tumor, such as lymphoma vaccine example is GTOP-99)(MyVax®).

Examples of chemotherapy agents for treating lymphoma or leukemia include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, BMS-345541, bortezomib (Velcade®), bryostatin 1, busulfan, carboplatin, campath-1H, CC-5103, carmustine, caspofungin acetate, clofarabine, cisplatin, Cladribine (Leustarin), Chlorambucil (Leukeran), Curcumin, cyclosporine, Cyclophosphamide (Cyloxan, Endoxan, Endoxana, Cyclostin), cytarabine, denileukin diftitox, dexamethasone, DT PACE, docetaxel, dolastatin 10, Doxorubicin (Adriamycin®, Adriblastine), doxorubicin hydrochloride, enzastaurin, epoetin alfa, etoposide, Everolimus (RAD001), fenretinide, filgrastim, melphalan, mesna, Flavopiridol, Fludarabine (Fludara), Geldanamycin (17-AAG), ifosfamide, irinotecan hydrochloride, ixabepilone, Lenalidomide (Revlimid®, CC-5013), lymphokine-activated killer cells, melphalan, methotrexate, mitoxantrone hydrochloride, motexafin gadolinium, mycophenolate mofetil, nelarabine, oblimersen (Genasense) Obatoclax (GX15-070), oblimersen, octreotide acetate, omega-3 fatty acids, oxaliplatin, paclitaxel, PD0332991, PEGylated liposomal doxorubicin hydrochloride, pegfilgrastim, Pentstatin (Nipent), perifosine, Prednisolone, Prednisone, R-roscovitine (Selicilib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, Temsirolimus (CCl-779), Thalidomide, therapeutic allogeneic lymphocytes, thiotepa, tipifarnib, Velcade® (bortezomib or PS-341), Vincristine (Oncovin), vincristine sulfate, vinorelbine ditartrate, Vorinostat (SAHA), vorinostat, and FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R-MCP (R-MCP).

The therapeutic treatments can be supplemented or combined with any of the abovementioned therapies with stem cell transplantation or treatment. One example of modified approach is radioimmunotherapy, wherein a monoclonal antibody is combined with a radioisotope particle, such as indium In 111, yttrium Y 90, iodine I-131. Examples of combination therapies include, but are not limited to, Iodine-131 tositumomab (Bexxar®), Yttrium-90 ibritumomab tiuxetan (Zevalin®), Bexxar® with CHOP.

Other therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogeneic hematopoietic stem cell transplantation.

Thus, provided is a method of sensitizing a subject who (i) is substantially refractory to at least one chemotherapy treatment, (ii) is in relapse after treatment with chemotherapy, or (iii) develops disease persistence to existing chronic MPN therapy, or any combination thereof, wherein the method comprises administering to the subject an effective amount of a JAK inhibitor, and an effective amount of a PI3K inhibitor or a pharmaceutically acceptable salt thereof. A subject who is sensitized is a subject who is responsive to the treatment involving administration of a JAK inhibitor and a PI3K inhibitor, or who has not developed resistance to such treatment. In one aspect, the JAK inhibitor is Compound A or ruxolitinib or pharmaceutically acceptable salt thereof, and the PI3K inhibitor is Compound B, C, D, or E, or pharmaceutically acceptable salt thereof. In certain aspect, the JAK inhibitor is Compound A or ruxolitinib or pharmaceutically acceptable salt thereof, and the PI3K inhibitor is Compound F or pharmaceutically acceptable salt thereof. In other aspect, the JAK inhibitor is Compound A or ruxolitinib or pharmaceutically acceptable salt thereof, and the PI3K inhibitor is Compound D1, D2, D3, D4, D5, D6, D7, D8, D9, E1, E2, E3, E4, E5, E6, E7, E8, or E9, or pharmaceutically acceptable salt thereof. In additional aspect, the JAK inhibitor is Compound A or pharmaceutically acceptable salt thereof, and the PI3K inhibitor is Compound B. In some additional aspect, the JAK inhibitor is Compound A or a pharmaceutically acceptable hydrochloride salt thereof, and the PI3K inhibitor is Compound B. In further aspect, the JAK inhibitor is ruxolitinib or pharmaceutically acceptable salt thereof, and the PI3K inhibitor is Compound B. In some further aspect, the JAK inhibitor is ruxolitinib or a pharmaceutically acceptable phosphate salt thereof, and the PI3K inhibitor is Compound B.

The treatment involving administration of the JAK inhibitor and the PI3Kδ inhibitor, can also sensitize, or restore sensitivity of, cells that may otherwise be resistant, have developed resistance, or not responsive, to killing or apoptosis by chemotherapy treatments or by administration of a JAK inhibitor alone. The cells that are sensitized, or have restored sensitivity, are the diseased cells that are responsive to the treatment involving administration of a JAK inhibitor and a PI3K inhibitor. In some embodiments, the administration of a JAK inhibitor and a PI3K inhibitor sensitizes, or restores sensitivity of, such MF cells by increasing the level of reduction in cell viability. In certain embodiments, the level of reduction in cell viability is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to contact with only a JAK inhibitor alone. Also, the level of reduction in cell viability may be increased by between 10% and 99%, between 10% and 90%, between 10% and 80%, between 10% and 70%, between 20% and 99%, between 20% and 90%, between 20% and 80%, between 25% and 95%, between 25% and 90%, between 25% and 80%, between 25% and 75%, or between 30% and 90%.

Treatment

As used herein, treatment or treating is an approach for obtaining beneficial or desired results including clinical results. For example, beneficial or desired clinical results may include one or more of the following: (i) decreasing one more symptoms resulting from the disease; (ii) diminishing the extent of the disease, stabilizing the disease (e.g., preventing or delaying the worsening of the disease); (iii) preventing or delaying the spread (e.g., metastasis) of the disease; (iv) preventing or delaying the occurrence or recurrence of the disease, delay or slowing the progression of the disease; (v) ameliorating the disease state, providing a remission (whether partial or total) of the disease, decreasing the dose of one or more other medications required to treat the disease; (vi) delaying the progression of the disease, increasing the quality of life, and/or (vii) prolonging survival.

The administration of a JAK inhibitor, such as Compound A or ruxolitinib or pharmaceutically acceptable salt thereof, and a PI3K-δ inhibitor, such as Compound B, Compound C, Compound D, or Compound E or pharmaceutically acceptable salts thereof, decreases the severity of the disease. Also, the administration of a JAK inhibitor, such as Compound A or ruxolitinib or pharmaceutically acceptable salt thereof, and a PI3K-δ inhibitor, such as Compound F, Compound D1-D9, or Compound E1-D9 or pharmaceutically acceptable salts thereof, decreases the severity of the disease. The decrease in the severity of the disease may be assessed by chemokine levels (e.g., CCL2, CCL3, CCL4, CCL22) by the methods described herein.

Also, the administration of one or more therapeutic agent, including a JAK inhibitor and/or a PI3K-δ inhibitor, may reduce the severity of one or more symptoms associated with cancer or myeloproliferative disorder by at least about any of 10%, 20%, 30%, 40%, 50%, 60%, 70%, 80%, 90%, 95% or 100% compared to the corresponding one or more symptoms in the same subject prior to treatment or compared to the corresponding symptom in other subjects not receiving such treatment.

As used herein, delaying the development of a cancer or myeloproliferative disease means to defer, hinder, slow, retard, stabilize, and/or postpone development of the disease. The delay can be of varying lengths of time, depending on the history of the disease and/or subject being treated. As is evident to one of skill in the art, a sufficient or significant delay can, in effect, encompass prevention, in that the individual does not develop the disease. A method that delays development of cancer or myeloproliferative disorder is a method that reduces probability of disease development in a given time frame and/or reduces the extent of the disease in a given time frame, when compared to not using the method. Such comparisons are typically based on clinical studies, using a statistically significant number of subjects. Disease development can be detectable using standard methods, such as routine physical exams, blood draw, mammography, imaging, or biopsy. Development may also refer to disease progression that may be initially undetectable and includes occurrence, recurrence, and onset.

The methods provided herein may be used to treat the growth or proliferation of cancer cells or myeloproliferative disease cells. By way of example, the cancer cells are of hematopoietic origin, myeloid, erythroid, megakaryocytic, or granulocytic, progenitors.

Also provided herein are the methods for decreasing cell viability in diseased cells in a human, comprising administering to a JAK inhibitor or a PI3K inhibitor in amounts sufficient to detectably decrease cell viability in the diseased cells. The cell viability in the cancer cells after administering to the human, or contacting the diseased cells with, a JAK inhibitor and/or a PI3K inhibitor is decreased by at least 10%, at least 20%, at least 30%, at least 40%, at least 50%, at least 60%, at least 70%, at least 80%, or at least 90% compared to cell viability in the diseased cells in the absence of the inhibitors. In addition, the cell viability in diseased cells after administering to the human, or contacting the cancer cells with, a JAK inhibitor and a PI3K inhibitor is decreased by between 10% and 99%, between 10% and 90%, between 10% and 80%, between 20% and 90%, between 20% and 80%, between 20% and 70% compared to cell viability in cancer cells in the absence of the inhibitors. Any suitable methods, techniques and assays known in the art may be used to measure cell viability. For example, cell viability in cancer cells is determined by flow cytometry or immunoblotting with the use of suitable stains, dyes, polynucleotide, polypeptide, or biomarkers.

The application also provides methods for decreasing AKT phosphorylation, S6 phosphorylation, and/or ERK phosphorylation in diseased cells in a human, comprising administering to the human a JAK inhibitor or a PI3K inhibitor in amounts sufficient to detectably decrease AKT phosphorylation, S6 phosphorylation, and/or ERK phosphorylation in the diseased cells. By way of example, AKT, S6, and/or ERK phosphorylation in the diseased cells after treatment is decreased by at least 10%, at least 20%, at least 30%, at least 40%, at least 50%, at least 60%, at least 70%, at least 80%, or at least 90% compared to S6 phosphorylation in the diseased cells in the absence of the inhibitors. Additionally, AKT, S6 and/or ERK phosphorylation in the diseased cells after administering to the human, or contacting the cancer cells with, a JAK inhibitor and a PI3K inhibitor is decreased by between 10% and 99%, between 10% and 90%, between 10% and 80%, between 20% and 90%, between 20% and 80%, between 20% and 70% compared to AKT and/or S6 phosphorylation in diseased cells in the absence of the inhibitors. Any suitable methods, techniques and assays known in the art may be used to measure AKT phosphorylation, S6 phosphorylation, and ERK phosphorylation. For example, AKT phosphorylation, S6 phosphorylation, and/or ERK phosphorylation is determined by flow cytometry or immunoblotting with the use of suitable stains, dyes, polynucleotide, polypeptide, or biomarkers. Moreover, the application provides methods for decreasing STAT3 phosphorylation and/or STAT5 phosphorylation in diseased cells in a human, comprising administering to the human a JAK inhibitor or a PI3K inhibitor in amounts sufficient to detectably decrease STAT3 phosphorylation and/or STAT5 phosphorylation in the diseased cells. By way of example, STAT3 and/or STAT5 phosphorylation in the diseased cells after treatment is decreased by at least 10%, at least 20%, at least 30%, at least 40%, at least 50%, at least 60%, at least 70%, at least 80%, or at least 90% compared to S6 phosphorylation in the diseased cells in the absence of the inhibitors. Additionally, STAT3 and/or STAT5 phosphorylation in the diseased cells after administering to the human, or contacting the cancer cells with, a JAK inhibitor and a PI3K inhibitor is decreased by between 10% and 99%, between 10% and 90%, between 10% and 80%, between 20% and 90%, between 20% and 80%, between 20% and 70% compared to STAT3 and/or STAT5 phosphorylation in diseased cells in the absence of the inhibitors. Any suitable methods, techniques and assays known in the art may be used to measure STAT3 phosphorylation and/or STAT5 phosphorylation. For example, STAT3 phosphorylation and/or STAT5 phosphorylation is determined by flow cytometry or immunoblotting with the use of suitable stains, dyes, polynucleotide, polypeptide, or biomarkers.

Provided herein also are methods for decreasing chemokine production in a sample, comprising contacting the sample with a JAK inhibitor and a PI3K inhibitor in amounts sufficient to detectably chemokine production in the sample. The levels of chemokine production or expression after contact or administer with a JAK inhibitor and a PI3K inhibitor is decreased by at least 5%, at least 10%, at least 20%, at least 30%, at least 40%, at least 50%, at least 60%, at least 70%, at least 80%, or at least 90% compared to those in the cells in the absence of inhibitors. The chemokine includes but is not limited to CCL2, CCL3, CCL4, CCL22, CXCL12, CXCL13, tumor necrosis factor alpha, c-creative protein, or any combination thereof. Any suitable methods, techniques and assays known in the art may be used to determine the levels of the chemokines in a sample. For example, immunoassays (or immunological binding assays) may be used to qualitatively or quantitatively analyze the chemokine levels in a sample. A general overview of the applicable technology can be found in a number of readily available manuals, e.g., Harlow & Lane, Cold Spring Harbor Laboratory Press, Using Antibodies: A Laboratory Manual (1999) Immunoassays typically use an antibody that specifically binds to a protein or antigen of choice. The antibody may be produced by any of a number of means well known to those of skill in the art.

For in vitro or in vivo studies, the effect amount of Compounds A, B, C, D, E, or ruxolinitib may be adjusted according to the experimental condition. Similarly, the effect amount of Compounds D1, D2, D3, D4, D5, D6, D7, D8, D9, E1, E2, E3, E4, E5, E6, E7, E8, E9, or F, may be adjusted according to the experimental condition for in vitro or in vivo studies. For example, compounds may be used in the amount of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 5.0, 6.0, 7.0, 8.0, 9.0, or 10.0 M. In some studies, the in vitro doses of the compounds may be calculated to correspond to the clinical doses of the compounds. The calculation may consider various factors, such as protein binding and plasma concentration. By way of example, the in-vitro doses of about 1 and about 20 nM of ruxolitinib may correspond to the potential Cₘᵢₙ (i e minimum plasma concentration of compound), C. (i.e. maximum plasma concentration of compound), C_{average} (i.e. the average plasma concentration of compound) respectively detected in patients receiving ruxolitinib 15-25 mg, and the in-vitro doses of about 695 nM and about 272 nM of Compound A may correspond to the potential C. and C_{average}, respectively, detected in patients receiving Compound A at 300 mg twice a day. In another example, the in-vitro doses of about 74 nM, about 200 nM, and about 421 nM of Compound B may correspond to the potential Cₘᵢₙ, C_{average}, and Cₘₐₓ, respectively, detected in the patients receiving Compound B at 150 mg twice a day. It is understood that the in vitro doses may differ, depend on the assay condition and other calculation factors, and that the clinical doses may differ depend on the disease indication and the patient condition.

### Dosing Regimen, Order of Administration, and Route of Administration

As used herein, a therapeutically effective amount means an amount sufficient to modulate JAK/STAT and/or PI3K pathways, and thereby treat a subject (such as a human) suffering an indication, or to alleviate the existing symptoms of the indication. Determination of a therapeutically effective amount is within the capability of those skilled in the art, especially in light of the detailed disclosure provided herein. A therapeutically effective amount of a JAK inhibitor, such as Compound A or ruxolitinib or pharmaceutically acceptable salt thereof, and a therapeutically effective amount of PI3K inhibitor, such as Compound B, Compound C, Compound D, or Compound E and pharmaceutically acceptable salt thereof, may (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease.

The dosing regimen of the inhibitors according to the present application may vary depending upon the indication, route of administration, and severity of the condition, for example, depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled physician or practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

The formulation and route of administration chosen may be tailored to the individual subject, the nature of the condition to be treated in the subject, and generally, the judgment of the attending practitioner. For example, the therapeutic index of the inhibitors described herein may be enhanced by modifying or derivatizing the compound for targeted delivery to the diseased cells expressing a marker that identifies the cells as such. For example, the compounds can be linked to an antibody that recognizes a marker that is selective or specific for cancer cells, so that the compounds are brought into the vicinity of the cells to exert their effects locally, as previously described. See e.g., Pietersz et al., Immunol. Rev., 129:57 (1992); Trail et al., Science, 261:212 (1993); and Rowlinson-Busza et al., Curr. Opin. Oncol., 4:1142 (1992).

### Dosing Regimen

The therapeutically effective amount of a JAK inhibitor, such as Compound A or ruxolitinib or pharmaceutically acceptable salt thereof, or a PI3K inhibitor, such as Compound B, Compound C, Compound D, or Compound E or pharmaceutically acceptable salts thereof, may be provided in a single dose or multiple doses to achieve the desired treatment endpoint. As used herein, dose refers to the total amount of an active ingredient (e.g., Compound A, Compound B, Compound C, Compound D, Compound E, or pharmaceutically acceptable salts thereof) to be taken each time by a subject (e.g., a human).

Exemplary doses of the compound of the present application may be between about 0.01 mg to about 1500 mg, or between about 10 mg to about 500 mg, or between about 25 mg to about 400 mg, or between about 50 mg to about 350 mg, or between about 75 mg to about 300 mg, or between about 100 mg to about 200 mg, or about 10 mg, or about 15 mg, or about 20 mg, or about 25 mg, or about 30 mg, or about 40 mg, or about 50 mg, or about 60 mg, or about 75 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 475 mg, or about 500 mg. It should be understood that reference to about a value or parameter herein includes (and describes) embodiments that are directed to that value or parameter per se. For example, description referring to about x includes description of “x” per se.

Each and every variation of the doses of a JAK inhibitor, such as Compound A or ruxolitinib or pharmaceutically acceptable salt thereof, may be combined with each and every variation of the doses of a PI3K inhibitor, such as Compound B, Compound C, Compound D, Compound E or pharmaceutically acceptable salt thereof, as if each and every combination is individually described. Also, each and every variation of the doses of a JAK inhibitor, such as Compound A or ruxolitinib or pharmaceutically acceptable salt thereof, may be combined with each and every variation of the doses of a PI3K inhibitor, such as Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, Compound E9, or Compound F, or pharmaceutically acceptable salt thereof, as if each and every combination is individually described. For example, a 100 mg dose of a JAK inhibitor may be administered with a PI3K inhibitor at a dose of 10, 20, 25, 35, 40, 50, 75, 100, 125, 150, 175, 200, 225, 250, 275, 300, 325, 350, 375, or 400 mg. In additional example, a 200 mg dose of a JAK inhibitor may be administered with a PI3K inhibitor at a dose of 10, 20, 25, 35, 40, 50, 75, 100, 125, 150, 175, 200, 225, 250, 275, 300, 325, 350, 375, or 400 mg. Additional example includes that a 300 mg dose of a JAK inhibitor may be administered with a PI3K inhibitor at a dose of 10, 20, 25, 35, 40, 50, 75, 100, 125, 150, 175, 200, 225, 250, 275, 300, 325, 350, 375, or 400 mg. In one embodiment, 200 mg of Compound A and 100 mg of Compound B or 200 mg of Compound A and 150 mg of Compound B are used in the methods or present application. For example, a 15 mg dose of a JAK inhibitor may be administered with a PI3K inhibitor at a dose of 10, 20, 25, 35, 40, 50, 75, 100, 125, 150, 175, 200, 225, 250, 275, 300, 325, 350, 375, or 400 mg. In additional example, a 20 mg dose of a JAK inhibitor may be administered with a PI3K inhibitor at a dose of 10, 20, 25, 35, 40, 50, 75, 100, 125, 150, 175, 200, 225, 250, 275, 300, 325, 350, 375, or 400 mg. Additional example includes that a 25 mg dose of a JAK inhibitor may be administered with a PI3K inhibitor at a dose of 10, 20, 25, 35, 40, 50, 75, 100, 125, 150, 175, 200, 225, 250, 275, 300, 325, 350, 375, or 400 mg. In one embodiment, 15 mg of ruxolitinib and 150 mg of Compound B, 20 mg of ruxolitinib and 150 mg of Compound B, or 25 mg of ruxolitinib and 150 mg of Compound B are used in the methods or present application. In additional embodiment, 15 mg of ruxolitinib and 100 mg of Compound B, 20 mg of ruxolitinib and 100 mg of Compound B, or 25 mg of ruxolitinib and 100 mg of Compound B are used in the methods or present application. The doses may be administered once or twice daily.

In other embodiments, the methods provided comprise continuing to treat the subject (e.g., a human) by administering the doses of inhibitors or compounds at which clinical efficacy is achieved or reducing the doses by increments to a level at which efficacy can be maintained. In a particular embodiment, the methods provided herein comprise administering to the subject (e.g., a human) an initial daily dose of 20 mg to 200 mg of the compound, and increasing said dose to a total dosage of 50 mg to 400 mg per day over at least 6 days. In a further embodiment, the methods provided herein comprise administering to the subject (e.g., a human) an initial daily dose of 1 mg to 400 mg of the compound, and increasing said dose to a total dosage of 10 mg to 800 mg per day over at least 6 days. Optionally, the dosage can be further increased to about 150-750 mg per day. The dose(s) of Compound A, Compound B, Compound C, Compound D and/or Compound E, or pharmaceutically acceptable salts thereof, may be increased by increments until clinical efficacy is achieved. Also, the dose(s) of Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, Compound E9, Compound F, ruxolitinib, or pharmaceutically acceptable salts thereof, may be increased by increments until clinical efficacy is achieved. Increments of about 10 mg, 25 mg, about 50 mg, about 70 mg, about 100 mg, or about 125 mg, or about 150 mg, or about 200 mg, or about 250 mg, or about 300 mg can be used to increase the dose. The dose can be increased daily, every other day, two, three, four, five or six times per week, or once per week.

The frequency of dosing will depend on the pharmacokinetic parameters of the compounds administered and the route of administration. The dosing frequency for the JAK inhibitor may be the same or different from the dosing frequency for the PI3K inhibitor. The JAK inhibitor, such as Compound A or ruxolitinib or pharmaceutically acceptable salt thereof, is administered once a day or twice a day. Also, the PI3K inhibitor, such as Compounds B, C, D, E or a pharmaceutically acceptable salt thereof, is administered once a day or twice a day. In addition, Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, Compound E9, or Compound F, or a pharmaceutically acceptable salt thereof, is administered once a day or twice a day. The administration of the JAK inhibitor and the administration of PI3K inhibitor may be together or separately.

The dose and frequency of dosing also depend on pharmacokinetic and pharmacodynamic, as well as toxicity and therapeutic efficiency data. For example, pharmacokinetic and pharmacodynamic information about the compound of the present application can be collected through preclinical in vitro and in vivo studies, later confirmed in humans during the course of clinical trials. Thus, a therapeutically effective dose can be estimated initially from biochemical and/or cell-based assays. Then, dosage can be formulated in animal models to achieve a desirable circulating concentration range that modulates PI3Kδ and/or expression or activity. As human studies are conducted further information will emerge regarding the appropriate dosage levels and duration of treatment for various diseases and conditions.

Toxicity and therapeutic efficacy of Compound A and Compound B, and ruxolitinib and Compound B can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

Compounds A, B, C, D, E or pharmaceutically acceptable salts thereof may be administered under fed conditions. Similarly, Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, Compound E9, or Compound F, or pharmaceutically acceptable salts thereof may be administered under fed conditions. The term fed conditions or variations thereof refers to the consumption or uptake of food, in either solid or liquid forms, or calories, in any suitable form, before or at the same time when the compounds or pharmaceutical compositions thereof are administered. Compound may be administered to the subject (e.g., a human) within minutes or hours of consuming calories (e.g., a meal). By way of example, the JAK inhibitor and/or the PI3K inhibitor is administered to the subject (e.g., a human) within 5-10 minutes, about 30 minutes, or about 60 minutes consuming calories.

### Order of Administration

The order of administering according to the present application may also vary. The compounds may be administered sequentially (e.g., sequential administration) or simultaneously (e.g., simultaneous administration). For example, the JAK inhibitor is administered before the PI3K inhibitor, or the PI3K inhibitor is administered before the JAK inhibitor. Also, the JAK inhibitor and the PI3K inhibitor are administered simultaneously. Further, the administration of the compounds can be combined with supplemental doses.

Sequential administration or administered sequentially means that the inhibitors, compounds, or drugs are administered with a time separation of several minutes, hours, days, or weeks. Compounds may be administered with a time separation of at least 15 minutes, at least 30 minutes, at least 60 minutes, or 1 day, 2 days, 3 days, 4 days, 5 days, 6 days, or 7 days, or 2 weeks, 3 weeks, 4 weeks, 5 weeks, 6 weeks, 7 weeks, or 8 weeks. When administered sequentially, the compounds or drugs may be administered in two or more administrations, and the compounds or drugs are contained in separate compositions which may be contained in the same or different packages.

Simultaneous administration or administered simultaneously means that the inhibitors, compounds, or drugs are administered with a time separation of no more than a few minutes or seconds. Compounds are administered with a time separate of no more than about 15 minutes, about 10 minutes, about 5 minutes, or 1 minute. When administered simultaneously, the inhibitors, compounds or drugs are contained in separate compositions or the same composition.

The present application show that the administration of a JAK inhibitor and a PI3K inhibitor provide unexpected synergy or synergistic effect(s). As used herein, synergy or synergistic effects means the effect achieved when the active ingredients used together is greater than the sum of the effects that results from using the compounds separately or greater than the additive effects resulted from the compound alone. A synergistic effect may be attained when the active ingredients are: (1) co-formulated and administered or delivered simultaneously in a combined formulation; (2) delivered sequentially or simultaneously as separate formulations; or (3) by some other regimen. In certain embodiments, a synergistic effect may be attained when the compounds are administered or delivered sequentially, e.g., in separate tablets, pills or capsules, or by different injections in separate syringes.

### Modes of Administration

Compounds according to the present application may be administered by any conventional method, including parenteral and enteral techniques. Parenteral administration modalities include those in which the composition is administered by a route other than through the gastrointestinal tract, for example, intravenous, intraarterial, intraperitoneal, intramedullary, intramuscular, intraarticular, intrathecal, and intraventricular injections. Enteral administration modalities include, for example, oral, buccal, sublingual, and rectal administration. Transepithelial administration modalities include, for example, transmucosal administration and transdermal administration. Transmucosal administration includes, for example, enteral administration as well as nasal, inhalation, and deep lung administration; vaginal administration; and buccal and sublingual administration. Transdermal administration includes passive or active transdermal or transcutaneous modalities, including, for example, patches and iontophoresis devices, as well as topical application of pastes, salves, or ointments. Parenteral administration also can be accomplished using a high-pressure technique, e.g., POWDERJECT™.

By way of example, the JAK inhibitor and the PI3K inhibitor are independently administered orally, intravenously or by inhalation. In one embodiment, the JAK inhibitor is administered orally, once or twice, at a dosage of about 10 mg, about 20 mg, about 25 mg, about 30 mg, about 40 mg, about 50 mg, about 75 mg, about 100 mg, about 150 mg, about 200 mg, about 225 mg, about 250 mg, about 275 mg, about 300 mg, about 350 mg, about 400 mg, about 450 mg, about 500 mg, about 550 mg, or about 600 mg. In other embodiment, the PI3K inhibitor is administered orally, once or twice, at a dosage of about 50 mg, about 100 mg, about 150 mg, about 200 mg, about 225 mg, about 250 mg, about 275 mg, about 300 mg, about 350 mg, about 400 mg, about 450 mg, about 500 mg, about 550 mg, or about 600 mg. In additional embodiment, the JAK inhibitor (such as Compound A or ruxolitinib or a pharmaceutically acceptable salt thereof) is administered orally, once or twice, at a dosage of about 15 mg, about 20 mg, about 25 mg, about 125 mg, about 200 mg, about 250 mg, or about 300 mg. In some additional embodiment, the PI3K inhibitor (such as Compound B, Compound C, Compound D, Compound E, Compound F, Compound D1, Compound D2, Compound D3, Compound D4, Compound D5, Compound D6, Compound D7, Compound D8, Compound D9, Compound E1, Compound E2, Compound E3, Compound E4, Compound E5, Compound E6, Compound E7, Compound E8, or Compound E9 or a pharmaceutically acceptable salt thereof) is administered orally, once or twice, at a dosage of about 1 mg, about 2 mg, about 5 mg, about 10 mg, 15 mg, about 20 mg, about 25 mg, about 50 mg, about 100 mg, about 150 mg, about 200 mg, about 250 mg, about 300 mg, or about 400 mg.

### Pharmaceutical Compositions

The one or more therapeutic agent such as JAK inhibitor and/or the PI3K inhibitor can each be administered or provided as the neat chemical, but it is typical, and preferable, to administer or provide the compounds in the form of a pharmaceutical composition or formulation. Accordingly, provided are pharmaceutical compositions that include the compound within the present application and a biocompatible pharmaceutical vehicle (e.g., carrier, adjuvant, and/or excipient). The composition can include the compounds as the sole active agent(s) or in combination with other agents, such as oligo- or polynucleotides, oligo- or polypeptides, drugs, or hormones mixed with one or more pharmaceutically acceptable vehicles. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

The compounds may be administered in the same or separate formulations. The pharmaceutical composition comprises the active ingredient or the compound of the present application and at least one pharmaceutically acceptable vehicle. Techniques for formulation and administration of pharmaceutical compositions can be found in Remingtons Pharmaceutical Sciences, 18th Ed., Mack Publishing Co, Easton, Pa., 1990; and Modern Pharmaceutics, Marcel Dekker, Inc. 3rd Ed. (G. S. Banker & C. T. Rhodes, Eds.). The pharmaceutical compositions described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The pharmaceutical compositions can be formulated to contain suitable pharmaceutically acceptable vehicles, which may include, for example, inert solid diluents and fillers, diluents, including sterile aqueous solution and various organic solvents, permeation enhancers, solubilizers and adjuvants. For example, the pharmaceutical compositions may comprise pharmaceutically acceptable carriers, and optionally can comprise excipients and auxiliaries that facilitate processing of the compound or active ingredient into preparations that can be used pharmaceutically. In another example, the pharmaceutical compositions may comprise pharmaceutically acceptable carriers, and optionally can comprise excipients and auxiliaries that facilitate processing of the compound or the active ingredient into preparations that can be used pharmaceutically. The mode of administration generally determines the nature of the carrier. For example, formulations for parenteral administration can include aqueous solutions of the active compounds in water-soluble form. Carriers suitable for parenteral administration can be selected from among saline, buffered saline, dextrose, water, and other physiologically compatible solutions. In one embodiment, carriers for parenteral administration include physiologically compatible buffers such as Hankss solution, Ringers solution, or physiologically buffered saline. For tissue or cellular administration, penetrants appropriate to the particular barrier to be permeated are used in the formulation. Such penetrants are generally known in the art. For preparations including proteins, the formulation can include stabilizing materials, such as polyols (e.g., sucrose) and/or surfactants (e.g., nonionic surfactants), and the like.

Alternatively, formulations for parenteral use can include dispersions or suspensions prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, dextran, and mixtures thereof. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the inhibitors or the compounds also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

In some embodiments, oral formulations include tablets, dragees, and gelatin capsules. These preparations can contain one or more excipients including but not limited to: (i) diluents, such as microcrystalline cellulose and sugars, including lactose, dextrose, sucrose, mannitol, or sorbitol; (ii) binders, such as sodium starch glycolate, croscarmellose sodium, magnesium aluminum silicate, starch from corn, wheat, rice, potato, etc.; (iii) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen; (iv) disintegrating or solubilizing agents such as cross-linked polyvinyl pyrrolidone, starches, agar, alginic acid or a salt thereof, such as sodium alginate, or effervescent compositions; (v) lubricants, such as silica, talc, stearic acid or its magnesium or calcium salt, and polyethylene glycol; (vi) flavorants and sweeteners; (vii) colorants or pigments, e.g., to identify the product or to characterize the quantity (dosage) of active compound; and (viii) other ingredients, such as preservatives, stabilizers, swelling agents, emulsifying agents, solution promoters, salts for regulating osmotic pressure, and buffers.

Gelatin capsules may include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragee cores may be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

### Articles of Manufacture and Kits

Compositions (including, for example, formulations and unit dosages) comprising the inhibitors or the compounds can be prepared and placed in an appropriate container, and labeled for treatment of an indicated condition. Accordingly, provided is also an article of manufacture, such as a container comprising a unit dosage form of the compound, and a label containing instructions for use of the compounds. In some embodiments, the article of manufacture is a container comprising (i) a unit dosage form of a JAK inhibitor and one or more pharmaceutically acceptable carriers, adjuvants or excipients; and (ii) a unit dosage form of a PI3K inhibitor and one or more pharmaceutically acceptable carriers, adjuvants or excipients.

As used herein, unit dosage form refers to physically discrete units, suitable as unit dosages, each unit containing a predetermined quantity of active ingredient, or compound which may be in a pharmaceutically acceptable carrier. One of skill in the art would recognize that the unit dosage form may vary depending on the mode of administration. Exemplary unit dosage levels for a human subject may be between about 0.01 mg to about 1000 mg, or between 10 mg to about 500 mg, or between about 25 mg to about 300 mg, or between about 50 mg to about 200 mg, or about 25 mg, about 50 mg, about 75 mg, about 100 mg, about 125 mg, or about 150 mg, or about 175 mg, about 200 mg, or about 250 mg, about 300 mg, about 350 mg, about 400 mg, about 500 mg, or about 600 mg. Other exemplary unit dosage levels for a human subject may be between about 1 mg to about 200 mg, or between about 1 mg to about 50 mg, or between about 1 mg to about 25 mg, or between about 1 mg to about 15 mg, or between about 2 mg to about 25 mg, between about 2 mg to about 15 mg, or between about 2 mg to about 10 mg, or between about 5 mg to about 15 mg, or between about 5 mg to about 10 mg, or about 1 mg, or about 2 mg, or about 5 mg, or about 10 mg, or about 15 mg, or about 20 mg.

Kits also are contemplated. For example, a kit can comprise unit dosage forms of the compounds, and a package insert containing instructions for use of the composition in treatment of a medical condition. In some embodiments, the kits comprises (i) a unit dosage form of the JAK inhibitor and one or more pharmaceutically acceptable carriers, adjuvants or excipients; and (ii) a unit dosage form of the PI3K inhibitor and one or more pharmaceutically acceptable carriers, adjuvants or excipients. By way of example, the unit dosage form for both JAK inhibitor and PI3K inhibitor is a tablet. The instructions for use in the kit may be for treating a cancer or a myeloproliferative disorder, including but not limited to, acute lymphocytic leukemia (ALL), B-cell ALL, acute myeloid leukemia (AML), chronic lymphocytic leukemia (CLL), small lymphocytic lymphoma (SLL), multiple myeloma (MM), non-Hodgkins lymphoma (NHL), indolent NHL (iNHL), mantle cell lymphoma (MCL), follicular lymphoma, Waldenstroms macroglobulinemia (WM), B-cell lymphoma, or diffuse large B-cell lymphoma (DLBCL), polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS) and systemic mast cell disease (SMCD).

### Example

### Example 1

### Effects of Compound B to PI3K Isoforms and AKT Phosphorylation

The effects of Compound B on the activities of class I PI3K isoforms were measured using an in vitro biochemical enzyme assay at steady-state concentrations of adenosine triphosphate (ATP). Compound B is (S)-2-(1-((9H-purin-6-yl)amino)propyl)-5-fluoro-3-phenylquinazolin-4(3H)-one as described above.

A time resolved fluorescence resonance energy transfer (TR-FRET) assay was used to monitor the formation of 3,4,5-inositol triphosphate (PIP₃) molecule, as it competed with fluorescently labeled PIP₃ for binding to the GRP-1 pleckstrin homology domain protein. The Results show that Compound B was a selective inhibitor to PI3Kδ. The inhibition to PI3Kδ was 450-fold compared to PI3Kα, 210-fold compared to PI3Kβ, and 110-fold compared to PI3Kγ.

In addition, Compound B was examined for the effects on the PI3K signaling pathway by determining the levels of AKT and S6 phosphorylation with or without TPO activation. Two cell lines, BaF3/MPL and UT-7/TPO sensitive or responsive to TPO activation were used. The cells were starved (i.e. growing on medium having less FBS) in 0/1% FBS/RPMI for two hours before treated with 0.1, 1.0, or 2.0 μM of Compound B or vehicle (0.1% DMSO in RPMI) for 2 hours at 37° C. To examine the TPO-activated phosphorylation, the cells were then treated or activated with 50 ng/mL of human recombinant TPO (Peprotech) for 10 minutes at 37° C. The TPO activation or treatment may reflect the conditions in diseased cells as the PI3K pathway is activated by TPO in myelofibrosis. After treating with compound and/or TPO, the cells were collected, lysed by lysis buffer (Cell Signaling), separated by SDS-PAGE, and analyzed by the Western blot using antibodies specific to p-AKT Ser473 or pS6 Ser235/236 (Cell Signaling). The phosphorylation levels in treated cells were calculated and compared to those of untreated cells (i.e. vehicle as negative control).

The results showed that the cells treated with Compound B exhibited the reduced AKT (p-AKT Ser473) and S6 (p-S6RP Ser235/236) phosphorylation. The BaF3/MPL cells treated with 0.1, 1.0, or 2.0 μM of Compound B and TPO exhibited reduced p-AKT levels of 51%, 64%, or 67%, respectively, and reduced p-S6 levels of 24%, 27%, or 41%, respectively, of those in the cells treated with vehicle. Moreover, the U7-7/TPO cells treated with 0.1, 1.0, or 2.0 μM of Compound B and TPO exhibited reduced p-AKT levels of 11%, 44%, or 55%, respectively, and reduced S6 levels of 13%, 28%, or 48%, respectively, compared to those treated with vehicle.

### Example 2

### Expressions of PI3K Isoforms in Progenitor Cells from Myelofibrosis Patients

To examine the PI3K isoform expression, the CD34cells were isolated from peripheral blood from healthy individuals (subjects 1-2) and from myelofibrosis (MF) patients who had not received any prior treatment (i.e. naïve)(subjects 3-5), had chronically received ruxolitinib (subjects 6-10) or Compound A (N-(cyanomethyl)-4-2-(4-morpholinoandino)pyrimidin-4-yl)benzamide)(subject 11-13).

The CD34⁺ (CD34⁺/CD3⁻/CD14⁻/CD19⁻/CD66⁻) cells were labeled and sorted by FACSAria (Beckman-Dickenson). The cell lysates were analyzed by Simple Western using Peggy (ProteinSimple) and AUC was plotted to quantify the levels of PI3K isoforms. Recombinant PI3K proteins were used as positive controls, and GAPDH was used to normalize isoform expression to total proteins.

The results of the study were summarized in Table 1. Among all samples (i.e. healthy individuals, untreated and treated MF patients), the levels of PI3Kδ were the highest among four isoforms.

TABLE 1
Expressions of PI3K isoforms in the CD34+ cells from healthy
individuals and myelofibrosis patients.

| Subject | PI3Kα | PI3Kβ | PI3Kδ | PI3Kγ |
|---|---|---|---|---|
| 1 | 0 | 4700 | 32580 | 320 |
| 2 | 0 | 8300 | 39260 | 0 |
| 3 | 0 | 36250 | 131240 | 2025 |
| 4 | 2800 | 21310 | 119520 | 1500 |
| 5 | 0 | 21340 | 65120 | 660 |
| 6 | 0 | 17870 | 41390 | 0 |
| 7 | 0 | 17350 | 51490 | 0 |
| 8 | 0 | 7740 | 41620 | 0 |
| 9 | 0 | 20680 | 37975 | 0 |
| 10 | 0 | 14610 | 68630 | 1550 |
| 11 | 0 | 12040 | 55050 | 1050 |
| 12 | 0 | 27180 | 73280 | 1540 |

### Example 3

### Effects of PI3K Inhibitors on Cellular Signaling in Progenitor Cells from Myelofibrosis Patients

In this example, PBMCs were isolated from whole blood of myelofibrosis (MF) patients who had not received treatments (i.e. naïve patients) or received ruxolitinib (i.e. nix-treated patients). The cells were treated with 0.02, 0.2, or 2.0 μM of Compound B or vehicle (0.1% DMSO in 0.1% FBS/RPMI) for 2 hours at 37° C. The cells were then fixed, permeabilized, and stained for FACS analysis. Antibodies specific to p-AKT Ser473 and pS6RP Ser235/236 were used to detect AKT phosphorylation (p-AKT) and S6RP phosphorylation (p-S6RP) in CD34⁺/CD3⁻/CD14⁻/CD19/CD66⁻ (BD Biosciences) gated cells using flow cytometry. The percentage of basal (i.e. untreated with TPO) AKT and S6RP phosphorylation were normalized to vehicle control (i.e. no TPO values shown in Table 2). A two-tailed paired t-test (GraphPad Prism) was used to calculate p-values. Values of p<0.05 were considered significant.

All subjects had the JAK2V617F mutation. The basal levels of phosphorylation in the CD34⁺ (CD34⁺/CD3⁻/CD14⁻/CD19⁻/CD66⁻) cells without TPO activation are summarized in Table 2, and the p-values are summarized in Table 3. The results show that, compared to untreated progenitor MF cells, the cells treated with Compound B exhibited reduced levels of p-AKT (Table 2) and p-S6RP (data not shown). In addition, the cells treated with higher concentration of Compound B exhibited higher levels of reduction. Moreover, the reduced phosphorylation levels or PI3K signaling were observed in the cells from MF patients who had received or not received Compound E. This suggests that the Compound B caused a dose-dependent inhibition to PI3K signaling in myelofibrosis patients who were naïve or had chronic ruxolitnib treatment.

TABLE 2
The normalized percentage of basal AKT phosphorylation in progenitor
cells isolated from Naïve or rux-treated MF patients treatedwith
Compound B.

| | p-AKT | | | |
|---|---|---|---|---|
| Subject | 0 | 0.02 μM | 0.2 μM | 2 μM |
| Naïve-1 | 100 | 84 | 81 | 70 |
| Naïve-2 | 100 | NA | 72 | 47 |
| Naïve-3 | 100 | 99 | 64 | 55 |
| Naïve-4 | 100 | 102 | 127 | 96 |
| Naïve-5 | 100 | 83 | 75 | 66 |
| Naïve-6 | 100 | 88 | 76 | 66 |
| Rux-1 | 100 | 88 | 85 | 69 |
| Rux-2 | 100 | 89 | 78 | 77 |
| Rux-3 | 100 | 91 | 81 | 83 |
| Rux-4 | 100 | 89 | 82 | 84 |
| Rux-5 | 100 | 57 | 52 | 43 |
| Rux-6 | 100 | 96 | 87 | 98 |
| Rux-7 | 100 | 100 | 82 | 79 |

TABLE 3
The p-values of basal AKT and S6RP phosphorylation in
the progenitor cells isolated from naïve
or rux-treated MF patients treated with Compound B.

| | p-AKT | | | p-S6RP | | |
|---|---|---|---|---|---|---|
| | 0.02 | 0.2 | 2 | 0.02 | 0.2 | 2 |
| Subjects | μM | μM | μM | μM | μM | μM |
| Naïve | NS¹ | NS | 0.0047 | 0.0205 | 0.0129 | 0.0151 |
| Rux-treated | 0.005 | 0.0027 | 0.0099 | 0.08 | 0.0002 | 0.0001 |

¹NS: not significant

Also, PBMC cells were treated with Compound B and with TPO as described above. The percentage of TPO-activated AKT and S6RP phosphorylation were normalized to those of TPO-treated vehicle. The percentage of phosphorylation levels of TPO-treated cells are summarized in Table 4, and the p-values are summarized in Table 5. Similar to those without TPO treatment, the cells (from patients who were naive or not received ruxolitinib) treated with Compound B exhibited reduced levels of p-AKT and p-S6RP. Also, the inhibition to PI3K signaling was dose-dependent to Compound B.

TABLE 4
The normalized percentage of TPO-activated AKT and S6RP
phosphorylation in the progenitor cells from naïve
or rux-treated MF patients treated with Compound B.

| | | p-AKT | | | | | p-S6RP | | | |
|---|---|---|---|---|---|---|---|---|---|---|
| | No | | 0.02 | 0.2 | 2.0 | No | | 0.02 | 0.2 | 2.0 |
| Subject | TPO | 0 | μM | μM | μM | TPO | 0 | μM | μM | μM |
| Naïve-3 | 20 | 100 | 58 | 41 | 27 | 17 | 100 | 86 | 69 | 45 |
| Naïve-4 | 45 | 100 | 43 | 30 | 28 | 32 | 100 | 57 | 48 | 59 |
| Naïve-5 | 53 | 100 | 60 | 32 | 32 | 11 | 100 | 50 | 42 | 23 |
| Rux | 68 | 100 | 59 | 39 | 40 | 15 | 100 | 55 | 35 | 21 |
| Rux-3 | 53 | 100 | 60 | 52 | 29 | 27 | 100 | 79 | 56 | 45 |
| Rux-4 | 14 | 100 | 60 | 55 | 35 | 14 | 100 | 60 | 55 | 35 |
| Rux-5 | 54 | 100 | 78 | 57 | 39 | 6 | 100 | 59 | 50 | 36 |
| Rux-6 | 16 | 100 | 55 | 36 | 16 | 7 | 100 | 53 | 32 | 20 |
| Rux-7 | 13 | 100 | 57 | 33 | 16 | 5 | 100 | 62 | 44 | 31 |

TABLE 5
The p-values of TPO-activated AKT and S6RP phosphorylation
in MF progenitor cells treated with Compound B.

| | p-AKT | | | p-S6RP | | |
|---|---|---|---|---|---|---|
| | 0.02 | 0.2 | 2 | 0.02 | 0.2 | 2 |
| Subjects | μM | μM | μM | μM | μM | μM |
| Naïve | 0.013 | 0.003 | 0.0005 | NS¹ | 0.029 | 0.03 |
| Rux-treated | 0.0001 | 0.0001 | 0.0001 | 0.0002 | 0.0001 | 0.0001 |

¹NS: not significant

### Example 4

### Effects of Compounds C and D on AKT and S6PR Phosphorylation

Similar studies were conducted with PI3K inhibitors Compounds C and D. PBMC from MF patients had received ruxolitinib (rux) and MF patient had received Compound A. The cells were treated with Compounds C or D at 0, 20.0, 200.0, 2000.0 nM for 2 hours at 37° C. Cells were treated with TPO for 10 minutes. The percentage of basal p-AKT and p-S6RP levels were normalized to vehicle control and those of TPO-treated were normalized to TPO-treated vehicle control. The PI3K inhibitors Compounds C and D had the chemical names of (S)-2-(1-((9H-purin-6-yl)amino)ethyl)-6-fluoro-3-phenylquinazolin-4(3H)-one and (S)-2,4-diamino-6-(((5-chloro-8-fluoro-4-oxo-3-(pyridine-3-yl)-3,4-dihydroquinazolin-2-yl)(cyclopropyl)methyl)amino)pyrimidine-5-carbonitrile, respectively.

Results showing their effects in the TPO-untreated and TPO-treated cells are summarized in Tables 6 and 7, respectively. Similar to Compound B, Compounds C and D inhibited the PI3K signaling as shown by the reduced phosphorylation levels of AKT and S6RP in MF progenitor cells. Also, Compounds C and D inhibited p-AKT and p-S6RP in a dose dependent manner as higher concentrations of Compounds C and D resulted in higher reduction in AKT/S6RP phosphorylation or PI3K signaling. Both compounds caused inhibition or reduction in the PI3K signaling or AKT/S6RP phosphorylation.

TABLE 6
The percentage of p-AKT and p-S6RP in basal and TPO-
treated MF progenitor cells treated with Compound C.

| | Rux-treated Cells | | | | Compound A-treated Cells | | | |
|---|---|---|---|---|---|---|---|---|
| | Basal | | TPO | | Basal | | TPO | |
| | pAKT | pS6 | pAKT | pS6 | pAKT | pS6 | pAKT | pS6 |
| 0 nM | 100 | 100 | 100 | 100 | 100 | 100 | 100 | 100 |
| 20 nM | 88 | 65 | 65 | 84 | 89 | 82 | 65 | 66 |
| 200 nM | 90 | 59 | 53 | 62 | 82 | 75 | 59 | 70 |
| 2000 nM | 75 | 43 | 24 | 42 | 75 | 53 | 43 | 36 |
| No TPO | NA² | NA | 40 | 29 | NA | NA | 16 | 9 |

²NA: not applicable

TABLE 7
The percentage of p-AKT and p-S6RP in basal and TPO-
treated MF progenitor cells treated with Compound D.

| | Rux-treated Cells | | | | Compound A-treated Cells | | | |
|---|---|---|---|---|---|---|---|---|
| | Basal | | TPO | | Basal | | TPO | |
| | pAKT | pS6 | pAKT | pS6 | pAKT | pS6 | pAKT | pS6 |
| 0 nM | 100 | 100 | 100 | 100 | 100 | 100 | 100 | 100 |
| 20 nM | 83 | 58 | 58 | 48 | 87 | 58 | 49 | 74 |
| 200 nM | 85 | 50 | 50 | 39 | 57 | 48 | 41 | 55 |
| 2000 nM | 76 | 39 | 39 | 19 | 60 | 18 | 19 | 25 |
| No TPO | NA² | NA | 50 | 11 | NA | NA | 22 | 22 |

²NA: not applicable

### Example 5

### Effects of PI3K Inhibitor and/or JAK Inhibitor in MF Progenitor Cells

In this example, effects of PI3K inhibitors and JAK2 inhibitors on cell growth and apoptosis were examined. To measure the effects on cell growth, PBMCs were isolated from the whole blood of MF patients had received chronic ruxolitinib. The cells were stained and CD34+ cells (CD34⁺/CD3⁻/CD14⁻/CD19⁻/CD66⁻) were isolated via sorting using FACSAria. About 10,000 cells per 96-well plate were added in StemSpan SFEM II media containing StemSpan CC110 cytokine cocktail (STEMCELL technologies). The cells were treated with either 1.0 μM of Compound B, 0.5 μM of ruxolitinib, the combination of 1.0 μM of Compound B and 0.5 μM of ruxolitinib, or vehicle (0.1% DMSO). After 72 hours, cell growth was measured using CellTiter-Glo (Promega). Raw data from all subjects treated with Compound B and/or ruxolitinib, or vehicle were collected together and calculated for the p-values using two-tailed paired t-test (GraphPad).

As shown in Table 8, the cells treated with Compounds B and/or ruxolitinib exhibited reduced cell viability or cell growth. Higher percentage indicates more viable cells. The cells treated with both compounds had the highest inhibition effects. This suggests the combination of PI3K inhibitor (such as Compound B) and JAK inhibitor (such as ruxolitinib) resulted in increased cell inhibition. The p-values were calculated for each compound alone vs. the combination: p0.0001 for compound B compared to the combination, and, p0.0003 for ruxolitinib compared to the combination. A p-value of less than 0.5 was significant.

TABLE 8
The percentage of viable cells in MF progenitor cells treated with
Compounds B and/or ruxolitinib.

| | | 1 μM | 0.5 μM | 1 μM Compound B + |
|---|---|---|---|---|
| Sample | Vehicle | Compound B | ruxolitinib | 0.5 μM ruxolitinib |
| 1 | 100 | 73 | 45 | 25 |
| 2 | 100 | 68 | 23 | 13 |
| 3 | 100 | 73 | 36 | 24 |
| 4 | 100 | 89 | 62 | 40 |
| 5 | 100 | 69 | 48 | 29 |
| 6 | 100 | 87 | 74 | 52 |
| 7 | 100 | 51 | 75 | 26 |
| 8 | 100 | 65 | 38 | 17 |
| 9 | 100 | 62 | 54 | 24 |

To measure apoptosis, PBMCs from MF patients who had received chronic ruxolitnib or Compound A were stained and isolated for CD34+ cells (CD34⁺/CD3⁻/CD14⁻/CD19⁻/CD66⁻) via sorting using FACSAria. About 10,000 cells per 96-well were plated in StemSpan SEEM II media containing StemSpan CC110 cytokine cocktail (STEMCELL Technologies). The cells either 1.0 μM of Compound B, 0.5 μM of ruxolitinib, the combination of 1.0 μM of Compound B and 0.5 μM of ruxolitinib, or vehicle. After 72 hours, the cell death or apoptosis was measured by labeling cells with 7-AAD/Annexin-V (GuavaNexin) followed by FACS analysis. The p-values were calculated for each compound alone vs. the combination: p0.0001 for compound B compared to the combination and p0.0001 for ruxolitinib compared to the combination. A p-value of less than 0.5 is significant.

Table 9 summarizes the percentages of Annexin-V positive cells from the ruxolitinib-treated MF patients, and Table 10 summarizes the percentages of Annexin-V positive cells from the Compound A-treated patients (subjects 10-12 in Example 2). As Annexin-V labels apoptotic cells, higher percentage indicates more apoptotic cells, i.e. increased cell death. The results show that the cells (from the ruxolitinib-treated MF patients) treated with either Compound B or ruxolitinib exhibited induced apoptosis, and that the cells treated with both compounds exhibited the highest induction of apoptosis.

TABLE 9
The percentage of Annexin-V positive cells in the progenitor cells from
the ruxolitinib-treated MF patients treated with Compounds B and/or
ruxolitinib.

| | | 1 μM | 0.5 μM | 1 μM Compound B + |
|---|---|---|---|---|
| Sample | Vehicle | Compound B | ruxolitinib | 0.5 μM ruxolitinib |
| 1 | 24 | 31 | 42 | 52 |
| 2 | 11 | 14 | 22 | 26 |
| 3 | 27 | 31 | 49 | 57 |
| 4 | 21 | 24 | 35 | 44 |
| 5 | 63 | 68 | 71 | 79 |
| 6 | 51 | 55 | 57 | 63 |

TABLE 10
The percentage of Annexin-V positive cells in the
progenitor cells from the Compound A-treated MF patients
treated with Compounds B and/or ruxolitinib.

| | p-AKT | | | | | p-S6RP | | | | |
|---|---|---|---|---|---|---|---|---|---|---|
| | No | | 0.02 | 0.2 | 2.0 | No | | 0.02 | 0.2 | 2.0 |
| Subject | TPO | 0 | μM | μM | μM | TPO | 0 | μM | μM | μM |
| 10 | 52 | 100 | 51 | 51 | 30 | 13 | 100 | 64 | 42 | 25 |
| 11 | 52 | 100 | 56 | 49 | 24 | 77 | 100 | 61 | 52 | 30 |
| 12 | 88 | 100 | 74 | 71 | 55 | 82 | 100 | 69 | 54 | 34 |

In addition, the cells from MF patients are treated with Compounds B, C, or D in combination with Compound A. MF patients may be naïve (i.e. have not received any treatments) or have received JAK inhibitor such as ruxolitinib or Compound A. The cell viability and the apoptosis of the treated cells are measured as described above.

### Example 7

### Combination Treatment with PI3K Inhibitor and JAK Inhibitor

This study evaluates the efficacy and safety of combination treatment of Compound B and ruxolitinib in patients having primary myelofibrosis, post-polycythemia or post-essential thrombocythemia myelofibrosis. The patients may have progressive or relapsed disease, or disease persistence on maximum clinically tolerated ruxolitinib therapy. The patients with progressive disease have: (i) appearance of a new splenomegaly that is palpable at least 5 cm below LCM, (ii) more than or equal to 100% increase in palpable distance, below LCM, for baseline splenomegaly of 5-10 cm, or (iii) about 50% increase in palpable distance, below LCM, for baseline splenomegaly of 10 cm. Also, the patients with relapsed disease have: (i) below criteria for at least CI after achieving CR, PR, or CI, or Loss of anemia response persisting for at least 1 month, or (ii) loss of spleen response persisting for at least 1 month. Also, disease persistence is defined as patients who are receiving FDA-approved JAK inhibitor therapy who meet the following criteria: relapsed disease, stable disease, or progressive disease with palpable splenomegaly (of 5 cm) that persists for 8 weeks up until the screening visit.

The patients are administered with ruxolitinib at a stable dose of 20, 15, or 5 mg (based on platelet count) orally twice daily for 8 weeks before being administered with 100 mg of Compound B orally twice daily in continuous 28 day cycles (1 cycle=28 days). After 2 cycles, patients may receive either 100 or 150 mg of Compound B orally twice daily. The patients continue to receive ruxolitinib, orally twice daily, at the same dose as pre-Compound B administration. The minimum duration of the study is 6 months.

Plasma concentration of Compound B is measured at trough (i.e. pre-dose) and peak (i.e., 1.5 hours post-dose) time points. At the end of each cycle, patients are evaluated at the end of each cycle for response rate, symptom burden, bone marrow fibrosis, and molecular responses. Response rate is defined as better than stable disease (including clinical improvement, partial improvement, or complete Improvement, spleen response, anemia response, symptoms response) according to criteria by International Working Group for Myelofibrosis Research and Treatment. The MF-associated symptomatic burden is determined by the Myeloproliferative Neoplasm Symptom Assessment Form, and bone marrow fibrosis is determined by European Fibrosis Scoring System. Blood samples are used to determine phosphorylation of the PI3K/AKT and other phosphorylated signaling intermediates (e.g. AKT, S6, STAT3, STAT5, ERK, NFkB), genetic mutation (e.g. JAK2V617F), and levels of systemic cytokines and chemokines (e.g. IL-6, IL-1RA, IL-1B, IL-2, FGF, MIP1b, TNFα, CCL3, CCL4, CXCL12, CXCL13).

Similar studies are conducted to evaluate the efficacy and safety of combination treatment of Compounds A and B in patients having primary myelofibrosis, post-polycythemia or post-essential thrombocythemia myelofibrosis.

### Example 8

### Effect of PI3K Inhibitor and JAK Inhibitor on the PI3K/AKT and the JAK/STAT5 Pathways

In this study, PBMCs were isolated from the whole blood from five MF patients receiving chronic ruxolitinib treatment (nix 1-nix 5). The cells were treated with either vehicle, ruxolitinib, and/or Compound B for 2 hours then stimulated with TPO (50 ng/mL) for 10 minutes. Ruxolitinib at the dose of 1 or 20 nM and Compound B at the dose of 45, 200, or 700 nM were used. The in vitro doses of 20 nM and 1 nM may correspond to the Cₘₐₓ and the Cₘᵢₙ, respectively, in the patients receiving ruxolitinib 15 mg twice a day.

The treated cells were then fixed, permeabilized and stained for FACS analysis using FACSCalibur analyzed using BD FACSDiva software. For analysis of the PI3K/AKT pathway, antibodies specific to p-S6RP were used to quantify the proportion of phosphorylated S6RP (p-S6RP) and in TPO-stimulated CD34⁺ (DAPI)/CD3⁻ (pacific blue)/CD14⁻ (pacific blue)/CD19⁻ (pacific blue)/CD66⁻ (pacific blue) gated CD34 cells using flow cytometry. For the JAK/STAT5 pathway, antibodies specific to p-STAT5 were used to quantify the proportion of phosphorylated STAT5 (p-STAT5) in TPO-stimulated CD34⁺ (DAPI)/CD3⁻ (pacific blue)/CD14⁻ (pacific blue)/CD19⁻ (pacific blue)/CD66⁻ (pacific blue) gated CD34 cells using flow cytometry. P-values were determined by comparing the group treated ruxolitinib alone to the group treated with both ruxolitinib and Compound B. The results were summarized in Tables 11 and 12.

As shown in Table 11, compared to those of the cells treated with the vehicle, the p-S6RP levels were decreased (i.e. p-S6RP was inhibited) by 40%, 52%, or 60% in the TP0-stimulated cells treated with compound B at 45, 200, or 700 nM, respectively. In the cells treated with ruxolitinib at 1 nM alone, the pS6RP levels were not inhibited. Also, in the cells treated with ruxolitinib at 20 nM alone, the p-S6RP levels were inhibited or reduced by 69%. In the cells treated with ruxolitinib at 20 nM and Compound B at 45, 200, or 700 nM, the p-S6RP levels were reduced or inhibited by 78%, 82%, and 86%, respectively. As shown in Table 12, pSTAT5 levels of the cells treated with both ruxolitinib and Compound B were decreased compared to those of the cells treated with either ruxolitinib or Compound B alone.

These results suggest that the addition of compound B to the MF patients who have received chronic ruxolitinib may provide additional benefit as Compound B may provide the target inhibition when ruxolitinib is at Cₘᵢₙ (i.e. 1 nM) and increase the target coverage when ruxolitinib is at Cₘₐₓ (i.e. 20 nM).

TABLE 11
The percentage of the p-S6RP levels in the CD34+ cells from the ruxolitinib-
treated MF patients treated with TPO, Compounds B, and/or ruxolitinib.

| | | | | | | | | | | | | | |
|---|---|---|---|---|---|---|---|---|---|---|---|---|---|
| Ruxolitnib (nM) | 0 | 0 | 1 | 20 | 0 | 0 | 0 | 1 | 1 | 1 | 20 | 20 | 20 |
| Compound B (nM) | 0 | 0 | 0 | 0 | 45 | 200 | 700 | 45 | 200 | 700 | 45 | 200 | 700 |
| TPO (50 ng/mL) | 0 | 50 | 50 | 50 | 50 | 50 | 50 | 50 | 50 | 50 | 50 | 50 | 50 |
| Rux-1 | 36 | 100 | 101 | 42 | 82 | 62 | 46 | 69 | 55 | 36 | 27 | 20 | 26 |
| Rux-2 | 13 | 100 | 78 | 25 | 58 | 46 | 48 | 46 | 37 | 34 | 15 | NA | 11 |
| Rux-3 | 28 | 100 | 92 | 22 | 49 | 38 | 32 | 43 | 39 | 26 | 17 | 17 | 12 |
| Rux-4 | 44 | 100 | 129 | 34 | 74 | 63 | 55 | 68 | 49 | 33 | 40 | 25 | 15 |
| Rux-5 | 7 | 100 | 75 | 28 | 36 | 33 | 17 | 36 | 20 | 16 | 9 | 10 | 6 |

NA: not available
Rux: MF patient receiving chronic ruxolitinib treatment

TABLE 12
The percentage of the p-STAT5 levels in the CD34+ cells from the ruxolitinib-
treated MF patients treated with TPO, Compounds B, and/or ruxolitinib.

| | | | | | | | | | | | | | |
|---|---|---|---|---|---|---|---|---|---|---|---|---|---|
| Ruxolitinib (nM) | 0 | 0 | 1 | 20 | 0 | 0 | 0 | 1 | 1 | 1 | 20 | 20 | 20 |
| Compound B (nM) | 0 | 0 | 0 | 0 | 45 | 200 | 700 | 45 | 200 | 700 | 45 | 200 | 700 |
| TPO (50 ng/mL) | 0 | 50 | 50 | 50 | 50 | 50 | 50 | 50 | 50 | 50 | 50 | 50 | 50 |
| Rux-1 | 72 | 100 | 83 | 77 | 92 | 93 | 88 | 75 | 82 | 76 | 63 | 66 | 60 |
| Rux-2 | 49 | 100 | 94 | 63 | 85 | 83 | 86 | 78 | 78 | 67 | 55 | 54 | 54 |
| Rux-3 | 70 | 100 | 97 | 74 | 95 | 91 | 82 | 96 | 82 | 76 | 63 | 70 | 59 |
| Rux-4 | 60 | 100 | 68 | 71 | 83 | 72 | 106 | 45 | 55 | 76 | 68 | 58 | 76 |
| Rux-5 | 39 | 100 | 91 | 62 | 92 | 90 | 78 | 81 | 73 | 66 | 61 | 55 | 47 |

Rux: MF patient receiving chronic ruxolitinib treatment

### Example 9

### The PI3K/AKT Pathway in Healthy Individuals and MF Patients

This study determined the raw mean fluorescence intensity (MFI) values of basal (i.e. no TPO stimulation) and TPO stimulated p-AKT and p-S6RP in healthy individuals (n=3), MF patients who had received ruxolitinib for more than 6 months (nix-chronic) (n=5), and MF patients who received no prior ruxolitinib treatments (i.e. nix-naïve) (n=4). The CD34+ cells were isolated using the same methods described above and analyzed by FACS at the photomultiplier tube (PMT) voltages on the FACS Calibur machine. Data was analyzed by BD FACSDiva software. The MFI values from unlabeled cells were subtracted from the MFI values of the samples.

Results are shown in Table 13. Compared to the nix-naïve patients and the healthy individual, the chronic-nix patients expressed increased levels of raw MFI of both basal (i.e. no TPO stimulation or 0 ng/mL TPO) and TPO stimulated p-S6RP. This suggests that the PI3K/AKT pathway is active in MF patients receiving ruxolitinib chronically. Compared to the nix-naïve patients, the chronic-nix patients had 2-fold increase of basal p-S6RP levels and 2.9-fold increase of TPO-stimulated p-S6RP levels. Also, compared to the rux-naïve patients, the chronic-nix patients had 1.4-fold increase of basal p-AKT levels and 1.5-fold increase of TPO-stimulated p-AKT levels. This suggests that the PI3K/AKT pathway may be activated in chronic-nix patients.

TABLE 13
The levels of basal and TPO-stimulated p-S6RP and p-AKT in the CD34
cells isolated from the healthy individuals and MF patients.

| | | | rux- | rux- | chronic- | chronic- |
|---|---|---|---|---|---|---|
| | healthy | healthy | naïve | naïve | rux | rux |
| TPO | 0 | 50 | 0 | 50 | 0 | 50 |
| p-AKT AVE¹ | 77 | 591 | 145 | 421 | 186 | 641 |
| p-AKT SD² | 19 | 36 | 26 | 100 | 76 | 214 |
| p-S6RP AVE¹ | 1789 | 27789 | 1887 | 12766 | 3707 | 37198 |
| p-S6RP SD² | 483 | 7627 | 148 | 6226 | 1061 | 3557 |

### Example 10

### Effect of PI3K Inhibitor and/or JAK Inhibitor on the JAK/STAT3 Pathway

This study examined the levels of phosphorylated STAT3 (pSTAT3)(i.e. STAT3 signaling) in Pfeiffer cells, a germinal center B-cell (GCB)-like diffuse large B-cell lymphoma (DLBCL) cell line. Among the non-Hodgkin lymphomas, DLBCL is a very heterogeneous disease. The activated B-cell (ABC)-DLBCL and GCB-DLBCL are subtypes of DLBCL. Increased STAT3 activation is frequently observed in both subtypes; increased pSTAT3 is reported in about 47% of ABC-DLBCL and about 30% of GCB-DLBCL patients (Blood 111:1515-1523, 2008; Journal Clinical Oncology 31:4520-4528, 2013). This suggests that the JAK/STAT3 pathway may be activated in ABC-DLBCL and GCB-DLBCL.

Pfeiffer cells were stimulated for 4 hours with 0, 1, 3, or 10 ng/mL of interleukin 6 (IL6) (n=1 per group) to activate STAT3 (i.e. increase the levels of pSTAT3). The cells were harvested, lysed and analyzed by Western blot. Antibodies specific for pSTAT3 (Tyr705) and total STAT3 were used to detect the levels of pSTAT3 and total STAT3, respectively. Raw pSTAT3 levels were quantified using densitometry software (Image Studio) and normalized to total STAT3 levels.

Compared to those of vehicle control (0 ng/mL IL6), the addition of IL6 to Pfeiffer cells increased the pSTAT3 levels (i.e. STAT3 activation) to 2.8-fold (1 ng/mL IL6), 3.8-fold (3 ng/mL IL6), and 2.5-fold (10 ng/mL IL6). The increased pSTAT3 levels induced by IL6 in this assay may correspond to the increased pSTAT3 levels (i.e. STAT3 activation) observed in ABC-DLBCL GCB-DLBCL patients (Journal Clinical Oncology 31:4520-4528, 2013).

Next, Pfeiffer cells were treated with IL6 (0 or 3 ng/mL), Compound A (0, 136, 272, or 695 nM), and/or Compound B (0, 74, 200, or 421 nM) (n=4 per group) for 96 hours. The doses in this assay may correspond to those used in a clinical setting. The in-vitro doses of 74 nM, 200 nM, and 421 nM of Compound B may correspond to the potential Cₘᵢₙ, C_{average}, and Cₘₐₓ, respectively, detected in the patients receiving Compound B at 150 mg twice a day for CLL treatment. Also, the in-vitro doses of 695 nM and 272 nM of Compound A may correspond to the potential Cₘₐₓ and C_{average}, respectively, detected in patients receiving Compound A at 300 mg twice a day for myelofibrosis treatment. In addition, the in-vitro dose of 136 nM Compound A may correspond to the potential C_{average} detected in patients receiving Compound A at 300 mg once a day.

Cellular viability was determined using the CellTiter-Glo Luminescent Cell Viability Assay (Promega). The percentage of viable cells was normalized to the groups that were not treated with Compound A or Compound B, and standard deviation was calculated. Results were summarized in Table 14. Compared to the cells not stimulated with IL6, the cells stimulated with IL6 exhibited reduced sensitivity to Compound B. Also, in the cells treated with IL6, Compound A, and Compound B, cell viability was decreased compared to those cells treated with IL6 and either compound alone. These results suggest that the combination treatment of PI3K-δ inhibitor (such as Compound B) and JAK inhibitor (such as Compound A) may provide potential therapeutic benefits to DLBCL patients, such as ABC-DLBCL or GCB-DLBCL. Moreover, the combination treatment of PI3K-δ inhibitor (such as Compound B) and JAK inhibitor (such as Compound A) may provide potential benefit in treating, preventing, or delaying resistance or relapse to existing treatment.

TABLE 14
The percentage of viable Pfeiffer cells treated with IL6, Compound A, and/or Compound B.

| IL6 | 0 ng/mL | | 3 ng/mL | | 3 ng/mL | | 3 ng/mL | | 3 ng/mL | |
|---|---|---|---|---|---|---|---|---|---|---|
| Compound A | 0 nM | | 0 nM | | 136 nM | | 272 nM | | 695 nM | |
| | AVG | SD¹ | AVG | SD¹ | AVG | SD¹ | AVG | SD¹ | AVG | SD¹ |
| 0 nM Compound B | 100 | 0 | 100 | 0 | 104 | 8 | 99 | 2 | 73 | 1 |
| 74 nM Compound B | 72 | 11 | 95 | 8 | 70 | 7 | 67 | 2 | 37 | 1 |
| 200 nM Compound B | 63 | 7 | 91 | 11 | 74 | 7 | 58 | 4 | 29 | 1 |
| 421 nM Compound B | 54 | 5 | 83 | 9 | 62 | 8 | 49 | 6 | 28 | 3 |

What is claimed is:

1. A method for treating a hyperproliferative disorder, comprising administering to a patient a therapeutic effective amount of JAK inhibitor and a therapeutic effective amount of PI3K inhibitor.

2. The method of claim 1, wherein the JAK inhibitor is selected from the group consisting of ruxolitinib, fedratinib, tofacitinib, baricitinib, lestaurtinib, pacritinib, decernotinib, XL019, AZD1480, INCB039110, LY2784544, BMS911543, NS018, GLPG0634, GLPG0788, or N-(cyanomethyl)-4-2-(4-morpholinoanilino)pyrimidin-4-yl)benzamide; or a pharmaceutically acceptable salt thereof.

3. The method of claim 1, wherein the JAK inhibitor is administered at a dose between 15 to 300 mg.

4. The method of claim 1, wherein the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl) (cyclopropyl)methyl)amino) pyrimidine-5-carbonitrile, (S)-3-(1-((9H-purin-6-yl)amino)ethyl)-8-chloro-2-phenylisoquinolin-1(2H)-one, (S)-2,4-diamino-6-(cyclopropyl(5,8-dichloro-4-oxo-3-(pyridin-3-yl)-3,4-dihydroquinazolin-2-yl)methylamino)pyrimidine-5-carbonitrile, (S)-2,4-diamino-6-(1-(5-chloro-3-(4-methylpyridin-3-yl)-4-oxo-3,4-dihydroquinazolin-2-yl)ethylamino) pyrimidine-5-carbonitrile, (S)-2,4-diamino-6-(1-(5-chloro-3-(5-fluoro-4-methylpyridin-3-yl)-4-oxo-3,4-dihydroquinazolin-2-yl)ethylamino)pyrimidine-5-carbonitrile, (S)-2,4-diamino-6-(1-(5-chloro-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-fluoropyridin-3-yl)-4-oxo-3,4-dihydroquinazolin-2-yl)ethylamino)pyrimidine-5-carbonitrile, (S)-2,4-diamino-6-(1-(5-methyl-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-methylpyridin-3-yl)-4-oxo-3,4-dihydroquinazolin-2-yl)ethylamino)pyrimidine-5-carbonitrile, (S)-2,4-diamino-6-((5-chloro-3-(5-fluoropyridin-3-yl)-4-oxo-3,4-dihydroquinazolin-2-yl)(cyclopropyl)methylamino) pyrimidine-5-carbonitrile, (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile, (S)-2,4-diamino-6-(1-(5,8-dichloro-4-oxo-3-(pyridin-3-yl)-3,4-dihydroquinazolin-2-yl)ethylamino) pyrimidine-5-carbonitrile, (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile, (S)-2,4-diamino-6-(1-(5-chloro-8-fluoro-3-(5-fluoropyridin-3-yl)-4-oxo-3,4-dihydroquinazolin-2-yl)ethylamino) pyrimidine-5-carbonitrile, (S)-2,4-diamino-6-((1-(5-chloro-3-(3-cyanophenyl)-4-oxo-3,4-dihydroquinazolin-2-yl)ethyl)amino)pyrimidine-5-carbonitrile, (S)-2,4-diamino-6-((1-(3-(3-cyanophenyl)-6-fluoro-4-oxo-3,4-dihydroquinazolin-2-yl)ethyl)amino)pyrimidine-5-carbonitrile, (S)-2,4-diamino-6-((1-(8-chloro-4-oxo-3-phenyl-3,4-dihydroquinazolin-2-yl)ethyl)amino)pyrimidine-5-carbonitrile, (S)-2,4-diamino-6-((1-(3-(3,5-difluorophenyl)-5,6-difluoro-4-oxo-3,4-dihydroquinazolin-2-yl)ethyl)amino)pyrimidine-5-carbonitrile, (S)-2,4-diamino-6-((1-(3-(3,5-difluorophenyl)-4-oxo-3,4-dihydroquinazolin-2-yl)propyl)amino) pyrimidine-5-carbonitrile, (S)-2,4-diamino-6-((1-(3-(3-cyanophenyl)-5-(difluoromethyl)-4-oxo-3,4-dihydroquinazolin-2-yl)ethyl)amino)pyrimidine-5-carbonitrile; or a pharmaceutically acceptable salt thereof.

5. The method of claim 1, wherein the PI3K inhibitor is administered at a dose between 10 mg and 300 mg.

6. The method of claim 1, further comprising one or more therapeutic agents selected from an Abl inhibitor, an ACK inhibitor, an A2B inhibitor, an ASK inhibitor, an Aurora kinase inhibitor, a BTK inhibitor, a BRD inhibitor, a c-Kit inhibitor, a c-Met inhibitor, a CAK inhibitor, a CaMK inhibitor, a CDK inhibitor, a CK inhibitor, a DDR inhibitor, an EGFR inhibitor, a FAK inhibitor, a Flt-3 inhibitor, a FYN inhibitor, a GSK inhibitor, a HCK inhibitor, a HDAC inhibitor, an IKK inhibitor, an IDH inhibitor, an IKK inhibitor, a KDR inhibitor, a LCK inhibitor, a LOX inhibitor, a LOXL inhibitor, a LYN inhibitor, a MMP inhibitor, a MEK inhibitor, a MAPK inhibitor, a NEK9 inhibitor, a NPM-ALK inhibitor, a p38 kinase inhibitor, a PDGF inhibitor, a PK inhibitor, a PLK inhibitor, a PK inhibitor, a PYK inhibitor, a SYK inhibitor, a TPL2 inhibitor, a STK inhibitor, a STAT inhibitor, a SRC inhibitor, a TBK inhibitor, a TIE inhibitor, a TK inhibitor, a VEGF inhibitor, a YES inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

7. The method of claim 1, wherein the administration of the JAK inhibitor is prior, concurrent, or subsequent to the administration of the PI3K inhibitor.

8. The method of claim 1, wherein the JAK inhibitor and the PI3K inhibitor are administered orally.

9. The method of claim 1, wherein said hyperproliferative disorder is myeloproliferative disorder selected from the group consisting of polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS), systemic mast cell disease (SMCD), Burkitts lymphoma, Hodgkins lymphoma, non-Hodgkins lymphoma (NHL), indolent non-Hodgkins lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestroms macroglobulinemia (WM), T-cell lymphoma, B-cell lymphoma, diffuse large B-cell lymphoma (DLBCL) including activated B-cell (ABC)-DLBCL and a germinal center B-cell (GCB)-like DLBCL, or marginal zone lymphoma (MZL).

10. The method of claim 1, wherein said patient is resistant or relapse to treatment of hyperproliferative disorder.

11. The method of claim 1, wherein said patient is resistant or relapse to the treatment of ruxolitinib.

12. The method of claim 1, wherein said patient has not previously been treated for hyperproliferative disorder.

13. A method for decreasing cell viability, decreasing proliferation, or increasing apoptosis, comprising contacting cells with an effective amount of JAK inhibitor and an effective amount of PI3K inhibitor.

14. The method of claim 13, wherein the JAK inhibitor is selected from the group consisting of ruxolitinib, fedratinib, tofacitinib, baricitinib, lestaurtinib, pacritinib, decernotinib, XL019, AZD1480, INCB039110, LY2784544, BMS911543, NS018, GLPG0634, GLPG0788; or N-(cyanomethyl)-4-2-(4-morpholinoanilino)pyrimidin-4-yl)benzamide; or pharmaceutically acceptable salts thereof.

15. The method of claim 13, wherein the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, AS252424, TGX221, TG100115, IC87114, ZSTK474, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino) pyrimidine-5-carbonitrile; (S)-3-(1-((9H-purin-6-yl)amino)ethyl)-8-chloro-2-phenylisoquinolin-1(2H)-one, (S)-2,4-diamino-6-(cyclopropyl(5,8-dichloro-4-oxo-3-(pyridin-3-yl)-3,4-dihydroquinazolin-2-yl)methylamino)pyrimidine-5-carbonitrile, (S)-2,4-diamino-6-(1-(5-chloro-3-(4-methylpyridin-3-yl)-4-oxo-3,4-dihydroquinazolin-2-yl)ethylamino) pyrimidine-5-carbonitrile, (S)-2,4-diamino-6-(1-(5-chloro-3-(5-fluoro-4-methylpyridin-3-yl)-4-oxo-3,4-dihydroquinazolin-2-yl)ethylamino)pyrimidine-5-carbonitrile, (S)-2,4-diamino-6-(1-(5-chloro-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-fluoropyridin-3-yl)-4-oxo-3,4-dihydroquinazolin-2-yl)ethylamino)pyrimidine-5-carbonitrile, (S)-2,4-diamino-6-(1-(5-methyl-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-methylpyridin-3-yl)-4-oxo-3,4-dihydroquinazolin-2-yl)ethylamino)pyrimidine-5-carbonitrile, (S)-2,4-diamino-6-((5-chloro-3-(5-fluoropyridin-3-yl)-4-oxo-3,4-dihydroquinazolin-2-yl)(cyclopropyl)methylamino) pyrimidine-5-carbonitrile, (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile, (S)-2,4-diamino-6-(1-(5,8-dichloro-4-oxo-3-(pyridin-3-yl)-3,4-dihydroquinazolin-2-yl)ethylamino) pyrimidine-5-carbonitrile, (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile, (S)-2,4-diamino-6-(1-(5-chloro-8-fluoro-3-(5-fluoropyridin-3-yl)-4-oxo-3,4-dihydroquinazolin-2-yl)ethylamino) pyrimidine-5-carbonitrile, (S)-2,4-diamino-6-((1-(5-chloro-3-(3-cyanophenyl)-4-oxo-3,4-dihydroquinazolin-2-yl)ethyl)amino)pyrimidine-5-carbonitrile, (S)-2,4-diamino-6-((1-(3-(3-cyanophenyl)-6-fluoro-4-oxo-3,4-dihydroquinazolin-2-yl)ethyl)aminolpyrimidine-5-carbonitrile, (S)-2,4-diamino-6-((1-(8-chloro-4-oxo-3-phenyl-3,4-dihydroquinazolin-2-yl)ethyl)amino)pyrimidine-5-carbonitrile, (S)-2,4-diamino-6-((1-(3-(3,5-difluorophenyl)-5,6-difluoro-4-oxo-3,4-dihydroquinazolin-2-yl)ethyl)aminolpyrimidine-5-carbonitrile, (S)-2,4-diamino-6-((1-(3-(3,5-difluorophenyl)-4-oxo-3,4-dihydroquinazolin-2-yl)propyl)amino) pyrimidine-5-carbonitrile, (S)-2,4-diamino-6-((1-(3-(3-cyanophenyl)-5-(difluoromethyl)-4-oxo-3,4-dihydroquinazolin-2-yllethyl)aminolpyrimidine-5-carbonitrile; or a pharmaceutically acceptable salt thereof.

16. The method of claim 13, wherein said cells are isolated from a subject having hyperproliferative disorder selected from the group consisting of polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS), systemic mast cell disease (SMCD), Burkitts lymphoma, Hodgkins lymphoma, non-Hodgkins lymphoma (NHL), indolent non-Hodgkins lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestroms macroglobulinemia (WM), T-cell lymphoma, B-cell lymphoma, diffuse large B-cell lymphoma (DLBCL) including activated B-cell (ABC)-DLBCL and a germinal center B-cell (GCB)-like DLBCL, and marginal zone lymphoma (MZL).

17. A pharmaceutical composition comprising a therapeutically effective amount of JAK inhibitor, a therapeutically effective amount of PI3K inhibitor, and a pharmaceutically acceptable excipient.

18. The method of claim 17, wherein the JAK inhibitor is selected from the group consisting of ruxolitinib, fedratinib, tofacitinib, baricitinib, lestaurtinib, pacritinib, decernotinib, XL019, AZD1480, INCB039110, LY2784544, BMS911543, NS018, GLPG0634, GLPG0788; or N-(cyanomethyl)-4-2-(4-morpholinoanilino)pyrimidin-4-yl)benzamide; or a pharmaceutically acceptable salt thereof.

19. The method of claim 17, wherein the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, AS252424, TGX221, TG100115, IC87114, ZSTK474, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2,4-diamino-6-(((5-chloro-8-fluoro-4-oxo-3-(pyridin-3-yl)-3,4-dihydroquinazolin-2-yl)(cyclopropyl)methyl)amino) pyrimidine-5-carbonitrile; (S)-3-(1-((9H-purin-6-yl)amino)ethyl)-8-chloro-2-phenylisoquinolin-1(2H)-one, (S)-2,4-diamino-6-(cyclopropyl(5,8-dichloro-4-oxo-3-(pyridin-3-yl)-3,4-dihydroquinazolin-2-yl)methylamino)pyrimidine-5-carbonitrile, (S)-2,4-diamino-6-(1-(5-chloro-3-(4-methylpyridin-3-yl)-4-oxo-3,4-dihydroquinazolin-2-yl)ethylamino) pyrimidine-5-carbonitrile, (S)-2,4-diamino-6-(1-(5-chloro-3-(5-fluoro-4-methylpyridin-3-yl)-4-oxo-3,4-dihydroquinazolin-2-yl)ethylamino)pyrimidine-5-carbonitrile, (S)-2,4-diamino-6-(1-(5-chloro-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-fluoropyridin-3-yl)-4-oxo-3,4-dihydroquinazolin-2-yl)ethylamino)pyrimidine-5-carbonitrile, (S)-2,4-diamino-6-(1-(5-methyl-4-oxo-3-(pyridin-3-yl)-3,4-dihydroquinazolin-2-yl)ethylamino)pyrimidine-5-carbonitrile, (S)-2,4-diamino-6-(1-(5-chloro-3-(5-methylpyridin-3-yl)-4-oxo-3,4-dihydroquinazolin-2-yl)ethylamino)pyrimidine-5-carbonitrile, (S)-2,4-diamino-6-((5-chloro-3-(5-fluoropyridin-3-yl)-4-oxo-3,4-dihydroquinazolin-2-yl)(cyclopropyl)methylamino) pyrimidine-5-carbonitrile, (S)-2,4-diamino-6-(1-(5-chloro-8-fluoro-4-oxo-3-(pyridin-3-yl)-3,4-dihydroquinazolin-2-yl)-2-cyclopropylethylamino)pyrimidine-5-carbonitrile, (S)-2,4-diamino-6-(1-(5,8-dichloro-4-oxo-3-(pyridin-3-yl)-3,4-dihydroquinazolin-2-yl)ethylamino) pyrimidine-5-carbonitrile, (S)-2,4-diamino-6-(1-(5,8-dichloro-3-(5-fluoropyridin-3-yl)-4-oxo-3,4-dihydroquinazolin-2-yl)ethylamino)pyrimidine-5-carbonitrile, (S)-2,4-diamino-6-(1-(5-chloro-8-fluoro-3-(5-fluoropyridin-3-yl)-4-oxo-3,4-dihydroquinazolin-2-yl)ethylamino) pyrimidine-5-carbonitrile, (S)-2,4-diamino-6-((1-(5-chloro-3-(3-cyanophenyl)-4-oxo-3,4-dihydroquinazolin-2-yBethyBamino)pyrimidine-5-carbonitrile, (S)-2,4-diamino-6-((1-(3-(3-cyanophenyl)-6-fluoro-4-oxo-3,4-dihydroquinazolin-2-yl)ethyl)amino)pyrimidine-5-carbonitrile, (S)-2,4-diamino-6-((1-(8-chloro-4-oxo-3-phenyl-3,4-dihydroquinazolin-2-yBethyBamino)pyrimidine-5-carbonitrile, (S)-2,4-diamino-6-((1-(3-(3,5-difluorophenyl)-5,6-difluoro-4-oxo-3,4-dihydroquinazolin-2-yl)ethyl)amino)pyrimidine-5-carbonitrile, (S)-2,4-diamino-6-((1-(3-(3,5-difluorophenyl)-4-oxo-3,4-dihydroquinazolin-2-yl)propyl)amino) pyrimidine-5-carbonitrile, (S)-2,4-diamino-6-((1-(3-(3-cyanophenyl)-5-(difluoromethyl)-4-oxo-3,4-dihydroquinazolin-2-yllethyl)aminolpyrimidine-5-carbonitrile; or a pharmaceutically acceptable salt thereof.

20. A kit comprising a pharmaceutical composition and a label, wherein the pharmaceutical composition comprising a therapeutically effective amount of JAK inhibitor, a therapeutically effective amount of PI3K inhibitor, and a pharmaceutically acceptable excipient.
